(19) United States
(12) Patent Application Publication
(10) Pub. No.: US 2009/0148342 A1
(43) Pub. Date: Jun. 11, 2009

(54) Hypochlorite Technology

(76) Inventors: Steven E. Bromberg, Oakland, CA (US); Bruce K. Bitowft, Oakland, CA (US); Elizabeth Crane, Oakland, CA (US); Maha El-Sayed, Oakland, CA (US); Daniel A. Huitt, Oakland, CA (US); Andreas Nguyen, Oakland, CA (US); Ricardo Ruiz de Gopegui, Oakland, CA (US); Doris S. Shieh, Oakland, CA (US); William L. Smith, Oakland, CA (US); Julie Timberman, Oakland, CA (US); Aram Garabedian, Fremont, CA (US); Lachelle Arnt, Pleasanton, CA (US); Elias A. Shaheen, Pleasanton, CA (US); Steven Bromberg, Pleasanton, CA (US); Vicki Friedman, Oakland, CA (US); Jennifer Fung, Oakland, CA (US); Jennifer C. Julian, Pleasanton, CA (US); Kenneth Vieira, Pleasanton, CA (US); Julie Wiesman, Pleasanton, CA (US); Timothy Kennedy, Oakland, CA (US); Chih Chiang, Oakland, CA (US); Kenneth L. Vieira, Oakland, CA (US); Maria G. Ochomogo, Oakland, CA (US); Scott Cumberland, Tracy, CA (US); Daniel Aaron Huitt, Esparto, CA (US); Diane Mellett, San Francisco, CA (US); Nikhil Dani, Pleasanton, CA (US); Maha Y. El-Sayed, Fremont, CA (US); Lafayette D. Foland, Dublin, CA (US); Jorge Robles, Stockton, CA (US); Elias A. Shaheen, Danville, CA (US)

Correspondence Address: THE CLOROX COMPANY, P.O. BOX 24305, OAKLAND, CA, 94623-1305, US

(21) Appl. No.: 11/927,288

(22) Filed: Oct. 29, 2007

(51) Int. Cl. A61L 2/20

(52) U.S. Cl. 422/37; 424/661; 422/123

(57) ABSTRACT

This invention generally relates to compositions and method of producing diluted hypohalous acid and hypohalous acid vapor. These compositions can be used to treat allergen containing surfaces, hard surfaces, food contact surfaces, hospital surfaces, food surfaces, kitchen surfaces, bathroom surfaces, human surfaces, animal surfaces, children's items, outdoor surfaces, soft surfaces, and medical instruments. These compositions can be converted to solid particulate or granular compositions. These compositions can be put into a variety of containers which preserve the stability. These compositions can be used to treat allergens and molds and as part of a mold detection system. These compositions can be dispersed into the air to enable microbiological control.

### CROSS REFERENCE TO RELATED APPLICATIONS

The present application is a continuation-in-part of Co-pending application Ser. No. 10/806,522 (Docket No. 340.182), which was filed Mar. 23, 2004, entitled “Methods for deactivating allergens and preventing disease”, and incorporated herein. The present application is a continuation-in-part of Co-pending application Ser. No. 10/870,096 (Docket No. 340.182B), which was filed Jun. 16, 2004, entitled “Complete Mold System”, which is a continuation-in-part of application Ser. No. 10/828,571 (Docket No. 340.182A, now abandoned), which was filed Apr. 20, 2004 entitled “Method of Diluting Hypochlorite”, and all incorporated herein. The present application is a continuation-in-part of Copending application Ser. No. 11/096,135 (Docket No. 340.182C), which was filed Mar. 31, 2005, entitled “Packaging for Dilute Hypochlorite”, which is a continuation-in-part of Co-pending application Ser. No. 10/838,571 (now abandoned), filed Apr. 23, 2004, which in turn is a continuation-in-part of Co-pending application Ser. No. 10/806,522, filed Mar. 23, 2004, all of which are incorporated within. The present application is a continuation-in-part of Co-pending application Ser. No. 11/130,070 (Docket No. 340.182D), which was filed May 16, 2005, entitled “Packaging for Dilute Hypochlorite”, which is a continuation-in-part of Co-pending application Ser. No. 10/828,571 (now abandoned), filed Apr. 20, 2004, all of which are incorporated within. The present application is a continuation-in-part of Copending application Ser. No. 11/111,012 (Docket No. 340.182E), which was filed Apr. 21, 2005, entitled “Dry Delivery Hypochlorite”, which is a continuation-in-part of Co-pending application Ser. No. 10/828,571 (now abandoned), filed Apr. 20, 2004, all of which are incorporated within. The present application is a continuation-in-part of Copending application Ser. No. 11/379,467 (Docket No. 340.182F), which was filed “Apr. 20, 2006, entitled “Humidifier Sanitization”, which is a continuation-in-part of Co-pending application Ser. No. 10/828,571 (now abandoned) published as U.S. Pat. App. 2005/0216,291, which was filed Apr. 20, 2004, entitled “Method for Diluting Hypochlorite”, which is a continuation-in-part of Co-pending application Ser. No. 10/806,522 published as U.S. Pat. App. 2005/0214386, which was filed Mar. 23, 2004, entitled “Methods for Deactivating Allergens and Preventing Disease”, all of which is incorporated herein. The present application is a continuation-in-part of Co-pending application Ser. No. 11/678,151 (Docket No. 340.182G), which was filed Feb. 23, 2007, entitled “Microbial Control Using Hypochlorous Acid Vapor”, which is a continuation-in-part of Co-pending application Ser. No. 11/111,012 published as U.S. Pat. App. 2005/0233900, which was filed Apr. 21, 2005, entitled “Dry Delivery Hypochlorite”, which is a continuation-in-part of Co-pending application Ser. No. 10/828,571 (now abandoned) published as U.S. Pat. App. 2005/0216,291, which was filed Apr. 20, 2004, entitled “Method for Diluting Hypochlorite”, all of which are incorporated herein. The present application is a continuation-in-part of Co-pending application Ser. No. 11/678,214 (Docket No. 340.182H), which was filed Feb. 23, 2007, entitled “Microbial Control with Reduced Chlorine”, which is a continuation-in-part of Co-pending application Ser. No. 11/111,012 published as U.S. Pat. App. 2005/0233900, which was filed Apr. 21, 2005, entitled “Dry Delivery Hypochlorite”, which is a continuation-in-part of Co-pending application Ser. No. 10/828,571 (now abandoned) published as U.S. Pat. App. 2005/0216,291, which was filed Apr. 20, 2004, entitled “Method for Diluting Hypochlorite”, all of which are incorporated herein. The present application is a continuation-in-part of Copending application Ser. No. 11/741,401 (Docket No. 340.1821) which was filed Apr. 27, 2007, entitled “Carriers for Hypochlorous Acid Vapor”, which is a continuation-in-part of Co-pending application Ser. No. 11/111,012, filed Apr. 21, 2005, which is a continuation-in-part of Co-pending application Ser. No. 10/828,571 (now abandoned), filed Apr. 20, 2004, all of which are incorporated by reference. The present application is a continuation-in-part of Co-pending application Ser. No. 11/762,254 (Docket No. 340.182J), which was filed Jun. 13, 2007, entitled “Method for Diluting Hypochlorite”, which is a continuation-in-part of Co-pending application Ser. No. 10/828,571 (now abandoned), which was filed Apr. 20, 2004, entitled “Method for Diluting Hypochlorite”, which is a continuation-in-part of Co-pending application Ser. No. 10/806,522, which was filed Mar. 23, 2004, entitled “Methods for deactivating allergens and preventing disease”, and both incorporated herein. The present application is a continuation-in-part and claims priority to co-pending application Ser. No. 11/379,135, which was filed Apr. 18, 2006, entitled “Thickened Dilute Hypochlorite” incorporated herein. The present application is a continuation-in-part and claims priority to co-pending application Ser. No. 11/277,642, which was filed Mar. 28, 2006, entitled “Antimicrobial Product Combination” incorporated herein.

### BACKGROUND OF THE INVENTION

1. Field of the Invention

This invention relates to methods for diluting hypohalous acid, hypohalous acid salt, and compositions containing these actives. The resulting compositions are useful for disinfecting (for example, water, environmental hard and soft surfaces, human and animal surfaces), sanitizing, sterilizing medical devices, controlling odor, deactivating allergens, and controlling mold. The resulting compositions can be applied by a variety of means, including vaporizing, spraying, soaking, and applying by means of an impregnated substrate. The resulting compositions can be applied on hard surfaces, soft surfaces and in the air.

This invention also relates to a complete mold system that provides consumers with tools for understanding, detecting, removing and preventing mold. The complete mold system will provide consumers with one comprehensive resource for taking care of their mold problem anywhere in the home. In addition to specific tools for detecting, removing, inhibiting/delaying and preventing mold, educational materials will guide consumers in a step-by-step manner on how best to take care of their mold problem.

This invention relates to packaging for dilute hypochlorite compositions, especially containers that provide stability to dilute hypochlorite and hypochlorous acid compositions. The invention also relates to dry powder forms and solid compositions containing hypohalite.

This invention relates to methods for delivering vapor phase hypohalous acid, dilute hypohalous acid, hypohalous acid salt, and compositions containing these actives into the air active or passive devices, such as using a humidifier. The invention also describes humidifiers, which deliver dilute hypohalous acid, hypohalous acid salt, and compositions containing these actives into the air vapor. The method and devices are useful for controlling microbiological contaminants and for treating the air, microbiologically contaminated surfaces, allergen containing surfaces, hard surfaces, food contact surfaces, hospital surfaces, food surfaces, kitchen surfaces, bathroom surfaces, human surfaces, animal surfaces, military equipment, transportation equipment, children's items, plant surfaces, seeds, outdoor surfaces, soft surfaces, air, wounds, and medical instruments.

This invention relates to shelving and displays for marketing a combination of antimicrobial products. More specifically, the invention comprises using a particular retail shelf display arrangement and particular products having a common antimicrobial active. The invention also relates to selling particular products having a common antimicrobial active in multi-packs.

2. Description of the Related Art

The compositions of the invention are generally non-hazardous, non-irritating and non-sensitizing to the skin, non-irritating to the eyes, not harmful if swallowed and show no evidence of mutagenic activity. Manufacturers of consumer goods often produce multiple products that are each focused on treating specific surfaces or one product that broadly treats multiple surfaces. Where antimicrobial products and antimicrobial product claims are involved, the products must be designed to pass rigorous testing protocol and be effective on each surface claimed. They must also meet consumer demands for safety and efficacy. Because of these limitations, it is difficult to design a single product to meet all the regulatory and consumer requirements for a variety of surfaces.

Consumers have recently become more concerned with mold due to increased media coverage of the effects of mold on health and home. In addition, research has shown that 100% of homes have mold, making mold relevant to all consumers. Although consumers know that mold is bad, they don't know how to take care of the mold problem. Several consumer products are marketed for removal of mold, however, these products do not deal with the identification and evaluation of mold or the safety requirements that may be necessary to deal with mold under certain conditions.

Mold presents special issues in treatment. Dust mite allergens, pet urine, and pet dander are non-living and, in general, are simple proteins. Prior art examples were able to modify dust mite allergens and other similar proteins so that they no longer complex with specific antibodies used in an ELISA test. These systems may not, however, denature living mold and pollen allergens, which are more complex than simple protein allergens. Mold and pollen allergens are living organisms containing protein, lipids and carbohydrates. Thus, treatments that are effective for some allergen problems may not be effective for molds and pollen. Additionally, prior art systems did not demonstrate the ability to modify the treated allergens so that they no longer generate any allergic response in animal systems.

Molds are usually not a problem indoors, unless mold spores land on a wet or damp spot and begin growing. Molds have the potential to cause health problems. Molds produce allergens (substances that can cause allergic reactions), irritants, and in some cases, potentially toxic substances (mycotoxins). Inhaling or touching mold or mold spores may cause allergic reactions in sensitive individuals. Allergic responses include hay fever-type symptoms, such as sneezing, runny nose, red eyes, and skin rash (dermatitis). Allergic reactions to mold are common. They can be immediate or delayed. Molds can also cause asthma attacks in people with asthma who are allergic to mold. In addition, mold exposure can irritate the eyes, skin, nose, throat, and lungs of both mold-allergic and non-allergic people. Molds can also produce organic toxins. These toxins include Aflatoxin B, Citrinin, Cyclosporin A, Deoxynivalenol, Emodin, Gliotoxin, Griseofulvin, Ochratoxin A, Patulin, Roridin A, Satratoxin H, Sterigmatocystin, T-2 toxin, Verrucarin A, and Endotoxins.

Generally, acaricides are used for controlling house dust mites. However, house dust mites, such as Dermatophagoides farinae, Dermatophagoides pteronyssinus, and so on can be the source of allergens even after dying and these dead bodies of house dust mites gradually decompose and release fine particles of allergens. As a result, controlling of house dust mites by applying acaricides is not always useful to remove allergens from the environment.

Treatments which modify the protein allergens from dust mites may be successful it preventing an allergic response. One measure of the success of these treatments is an in-vitro ELISA test which measures the binding of the modified proteins to enzyme-bound monoclonal antibodies. This test can show reduced binding which may or may not indicate a changed allergenic response. In-vivo test methods measure the allergenic response directly.

U.S. Pat. Appl. No. 2002/0179884 to Hoshino et al. and U.S. Pat. Appl. No. 2001/0048097 to Inui et al. disclose a method to modify binding of mite and pollen allergens above 90% efficiency using the ELISA method by treatment with rare earth metal salt in alcohol and other solvents for 5 hours. European Patent Applications 1,224,955 and 1,219,323 to Reckitt Benckiser disclose deactivants for dust mite feces. These include 6-isopropyl-m-cresol and a list of essential oils, organic compounds, and inorganic compounds. These deactivants were tested on household dust treated for 4 hours and then tested for binding response in an ELISA test for dust mite allergens. In general, the deactivants were not as effective as the control, tannic acid. They also revealed significant amounts of active allergens remaining for both tannic acid and the disclosed deactivants. PCT Application No. WO00/01429 to Hughes et al. discloses a device generating spray droplets with a unipolar charge from a composition containing allergen deactivants. The air particles remaining after treatment were tested under ELISA conditions for binding. Since the charged droplet device spraying of any composition would be expected to reduce airborne particles, the effect of the particular composition used is unclear. In addition, presumably many allergenic airborne particles remained. PCT Application No. WO01/013962 to Houlbrook discloses steam to denature substantially more allergens than would be denatured under normal laundry conditions. No data on the test method or effectiveness is disclosed.

WO02/28187 to Hasan et al. discloses Selkon states that low concentration of hypochlorite ions can reduce dust mite allergen binding up to 82% by an ELISA test after treatment for 1 hour. U.S. Pat. No. 6,428,801 to Suh et al. discloses that various formulations can reduce dust mite populations after treatment for an undetermined time.

U.S. Pat. Appl. No. 2004/0020007 to Lausevic describes a vacuum cleaner with a special attachment and a HEPA filter for removing mold. U.S. Pat. No. 6,716,885 to Twydell et al., U.S. Pat. No. 6,440,365 to Poye et al. found that the reduction in concentration of the chlorine compound in an attempt to ensure safety and prevent damage to objects involves complex compositions.

U.S. Pat. No. 5,342,597 to Tunison, III, U.S. Pat. No. 3,393,155 to Schutte et al. and U.S. Pat. No. 4,008,170 to Allan describe water dispersed in hydrophobic silica particles to give what is sometimes referred to as “dry water”. U.S. Pat. Appl. No. 2003/0160209 to Hoffman et al., electrolytically generated hypochlorite solutions thickened with Laponite clay. PCT Appl. No. WO97/11147 to Liciani describes the preparation of “dry oxone” from 1 N oxone solution and treated fumed silica. The “dry oxone” is useful in preventing collateral damage in detoxifying hazardous materials. U.S. Pat. No. 6,569,353 to Giletto et al. describes a dual system of persulfate and oxidant in a sorbent material and an activator in a sorbent material, where the two gels are mixed together to give a material for decontaminating toxic agents. The sorbent material is selected from silicon dioxide, silica gel, silicon oxyhydroxides, aluminum oxide, alumina gel, aluminum oxyhydroxides, aluminates, other metal oxides, other metal oxyhydroxides, clay minerals and mixtures thereof, preferably, fumed silica. U.S. Pat. No. 3,730,789 to Mueller et al. describes rocket propellant formed by gelling aqueous oxidants with silica gel.

U.S. Pat. Appl. No. 2003/0156980 to Fischer et al. produced thickened solutions of 2.7-3% hypochlorite thickened with a combination of clay and acrylic polymer. U.S. Pat. Appl. No. 2006/0011885 describes a thickened hypochlorite using fumed silica and optional additional abrasive cleaner, where clay is one of the disclosed thickeners. U.S. Pat. Appl. No. 2002/0179884 to Hoshino et al. found that applying a mist of dilute concentration hypochlorite solutions create difficulties in obtaining a formulation with satisfactory storage stability. That is, the activity would be reduced considerably due to the surrounding temperature, light (ultraviolet light), a third component adhered to a container, etc., a pigment present in a container material, and so on, and chlorine gas generation with decomposition of the chlorine compound. Thus, it has been difficult with a disinfecting deodorant comprising an aqueous solution of the chlorine compound to achieve sufficient disinfecting and deodorizing effects in such a low concentration range as to satisfy requirements for safety and the like describes inspecting a building for Stachybotris, applying hydrochloric acid, and heating the applied treatment. U.S. Pat. No. 5,395,541 to Carpenter et al. further finds that the composition is preferably from pH 9.5 to 11. If the pH is below 8, the disinfecting deodorant has a fear of generating chlorine gas with decomposition of the chlorine-containing oxidizing agent and fails to have sufficient storage stability. U.S. Pat. No. 5,281,280 to Lisowski et al. finds that concentrations below 2.75% are ineffective against mold, mildew and algae. U.S. Pat. No. 5,749,924, Mirch et al. discloses oleate and phosphate compositions for fabric and hard surfaces. U.S. Pat. No. 5,336,500 to Richter et al. discloses unsaturated monocarboxylic acid and benzoic acid for both hard and soft surfaces. PCT Pub. WO 97/30586 to Romano et al. discloses a disinfecting composition having terpene, phenolic, and peroxide for use on hard and soft surfaces. U.S. Pat. No. 5,591,395 to Schroeder et al. describes compositions containing propylene glycol for air sanitization that are not appropriate for treatment of hard and soft surfaces.

Potential uses for the inventive compositions and methods include for dishwashing, for example U.S. Pat. Appl. No. 2003/0216271 to Scheper et al.; for hospital environments and medical instruments, for example U.S. Pat. No. 6,632,347 to Buckley et al. and U.S. Pat. No. 6,126,810 to Fricker et al.; for wound healing, for example U.S. Pat. Appl. No. 2003/0185704 to Bernard et al. This is because loss of 100 ppm available chlorine in a 5% hypochlorite composition is usually not critical, but the same loss in a composition with 150 ppm available chlorine might be fatal. Hoshino lists several factors that affect the storage stability of dilute hypochlorite compositions, but offers no packaging solutions. U.S. Pat. No. 6,426,066 to Najafi et al. discloses disinfecting or sterilizing objects such as medical instruments, for example U.S. Pat. No. 6,623,695 to Malchesky et al.; for disinfecting and deodorizing the air, for example U.S. Pat. Appl. No. 2002/0179884 to Hoshino et al.; for water purification, for example U.S. Pat. No. 6,296,744 to Djeiranishvili et al.; for removal of mold and mildew, for example U.S. Pat. No. 5,281,280 to Lisowski et al describes containers for oxidized water, where glass containers were preferred over HDPE or Teflon®.

U.S. Pat. No. 6,586,063 to Albanesi et al. describes stable multilayer containers for dry delivery of concentrated hypochlorite. The preferred outer layer for the container was PP or PET. The preferred inner layer was LDPE or LLDPE. The multilayer container could also be stabilized against permeation of hypochlorite by including a barrier layer of MDPE, HDPE, or EVOH. U.S. Pat. App. No. 2003/0186827 to Makansi describes an aerosol container for concentrated hypochlorite. The preferred inner liner for the container is polyethylene or polypropylene. U.S. Pat. No. 5,080,826 to Colborn et al. describes containers for fragranced concentrated hypochlorite. The preferred container material is HDPE for its molding properties, rather than for stability. Colburn mentions various other additives, such as colorants, opacifying agents, antioxidants, and plasticizing agents, but there is no concern about these additives for hypochlorite stability.

No hypochlorite products currently exist in aerosol type containers or delivery devices which generate small droplet size. U.S. Pat. Appl. No. 2003/0186827 to Makansi describes an aerosol container for concentrated hypochlorite. The preferred inner liner for the container is polyethylene or polypropylene. Dilute hypochlorite presents even more difficulty in achieving sufficient stability. We have found the lined aerosol cans do not provide sufficient stability to dilute hypochlorite compositions. Makansi also describes an aerosol dispenser where the hypochlorite composition and the propellant are injected inside a flexible pouch. We have found that dilute hypochlorite compositions do not have sufficient stability in the same pouch with propellant.

Based on the prior art examples, the need exists for containers for dilute hypochlorite that can give suitable storage stability. Various novel containers and container materials for hypohalous acid, hypohalous acid salt, and compositions containing these actives. to deal with mold problems. However, the need still exists for a system to detect, remove and prevent mold problems. The complete mold system will empower consumers by providing a comprehensive solution that includes step-by-step guidelines for detecting and removing mold has been discovered.

### SUMMARY OF THE INVENTION

In accordance with the above objects and those that will be mentioned and will become apparent below, one aspect of the present invention is a method for producing a stable dilute composition, said composition selected from the group consisting of hypohalous acid, hypohalous acid salt, and combinations thereof, said method comprising the steps of:

preparing a first solution having an active halogen content of greater than about 0.5% as available chlorine; and

diluting said first solution with purified water to give a second solution;

wherein said second solution has an available chlorine concentration of between 40 ppm to about 400 ppm;

wherein said second solution retains at least 50% of the available chlorine concentration at a storage temperature of 120° F. over 27 days;

wherein said stable dilute composition does not contain additives selected from the group consisting of surfactants, alcohols, hydroxyacids, fragrances or combinations thereof.

In accordance with the above objects and those that will be mentioned and will become apparent below, one aspect of the present invention is a package for dilute hypohalous acid, hypohalous acid salt, and combinations thereof comprising:

a container;

a label; and

a composition within the container, said composition selected from the group consisting of hypohalous acid, hypohalous acid salt, and combinations thereof,

wherein said composition has an available chlorine concentration of between 1.0 ppm to about 1200 ppm;

wherein said container is selected from the group consisting of a trigger sprayer, a bag-in-can device, a plastic aerosol container, a dual delivery container, a dual chambered device, an expandable chamber device, a precompression trigger sprayer, a mechanically pressurized device, an ultrasonic sprayer, and combinations thereof.

In accordance with the above objects and those that will be mentioned and will become apparent below, one aspect of the present invention is a system for mold or allergen removal comprising:

a detection device for mold or allergen removal; and

a treatment device for mold or allergen removal.

In accordance with the above objects and those that will be mentioned and will become apparent below, one aspect of the present invention is a powder composition comprising:

greater than 10% water;

a compound selected from the group consisting of hypochlorite, hypochlorous acid, and combinations thereof, and

silica.

In accordance with the above objects and those that will be mentioned and will become apparent below, one aspect of the present invention is a method of controlling microbiological contaminants in a confined space comprising the steps of:

optionally, placing an object containing a microbiological contaminant in the confined space;

placing a composition comprising a source of hypohalous acid the confined space;

allowing hypohalous acid vapor from the source of hypohalous acid to control microbiologocal contaminants.

Further features and advantages of the present invention will become apparent to those of ordinary skill in the art in view of the detailed description of preferred embodiments below.

### BRIEF DESCRIPTION OF THE DRAWINGS

The foregoing aspects and others will be readily appreciated by the skilled artisan from the following description of illustrative embodiments when read in conjunction with the accompanying drawings, wherein:

FIG. 1 illustrates one embodiment of the invention;

FIG. 2 illustrates another embodiment of the invention;

FIG. 3 illustrates another embodiment of the invention;

FIG. 4 illustrates another embodiment of the invention;

FIG. 5 illustrates another embodiment of the invention;

FIG. 6 illustrates another embodiment of the invention; and

FIG. 7 illustrates another embodiment of the invention.

The invention is pointed out with particularity in the appended claims. The drawings are not necessarily to scale, emphasis instead generally being placed upon illustrating the principles of the invention. The advantages of the invention described herein, as well as further advantages of the invention, can be understood by references to the description taken in conjunction with the accompanying drawings.

### DETAILED DESCRIPTION OF THE INVENTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference. The citation of any document is not to be construed as an admission that it is prior art with respect to the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written. As used herein and in the claims, the term “comprising” is inclusive or open-ended and does not exclude additional unrecited elements, compositional components, or method steps. Accordingly, the term “comprising” encompasses the more restrictive terms “consisting essentially of” and “consisting of”.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active). For compositions on substrates the weight percent is of the cleaning composition alone, not accounting for the substrate weight, unless otherwise. Each of the noted cleaner composition components and substrates is discussed in detail below. All parts, ratios, and percentages herein, in the Specification, Examples, and Claims, are by weight and all numerical limits are used with the normal degree of accuracy afforded by the art, unless otherwise specified.

As used herein, the term “substrate” is intended to include any web, which is used to clean an article or a surface. Examples of cleaning sheets include, but are not limited to, mitts, webs of material containing a single sheet of material which is used to clean a surface by hand or a sheet of material which can be attached to a cleaning implement, such as a floor mop, handle, or a hand held cleaning tool, such as a toilet cleaning device.

As used herein, “wiping” refers to any shearing action that the substrate undergoes while in contact with a target surface. This includes hand or body motion, substrate-implement motion over a surface, or any perturbation of the substrate via energy sources such as ultrasound, mechanical vibration, electromagnetism, and so forth.

The term “cleaning composition”, as used herein, is meant to mean and include a cleaning formulation having at least one surfactant.

As used herein, the terms “nonwoven” or “nonwoven web” means a web having a structure of individual fibers or threads which are interlaid, but not in an identifiable manner as in a knitted web. Nonwoven webs have been formed from many processes, such as, for example, meltblowing processes, spunbonding processes, and bonded carded web processes.

The term “surfactant”, as used herein, is meant to mean and include a substance or compound that reduces surface tension when dissolved in water or water solutions, or that reduces interfacial tension between two liquids, or between a liquid and a solid. The term “surfactant” thus includes anionic, nonionic, cationic, amphoteric agents, zwitterionic surfactants and/or mixtures thereof.

As used herein, the term “microbiological contaminants” refers to any microbial contaminant. Example of microbiological contaminants include, but are not limited to, fungi, bacteria, viruses, Protista, prions, archaea, and molds, including mold spores. Examples of such microbiological contaminants include Stachybotrys Chartarum, Aspergillus niger, Absidia sp., Acrodorticm salmoneum, Aspergillus candies, anthrax, etc.

The composition can be used to control microbiological contaminants. The composition can be used as a disinfectant, sanitizer, and/or sterilizer. As used herein, the term “disinfect” shall mean the elimination of many or all pathogenic microorganisms on surfaces with the exception of bacterial endospores. As used herein, the term “sanitize” shall mean the reduction of contaminants in the inanimate environment to levels considered safe according to public health ordinance, or that reduces the bacterial population by significant numbers where public health requirements have not been established. At least 99% reduction in bacterial population within a 24 hour time period is deemed “significant.” As used herein, the term “sterilize” shall mean the complete elimination or destruction of all forms of microbial life and which is authorized under the applicable regulatory laws to make legal claims as a “Sterilant” or to have sterilizing properties or qualities.

The term “surface” refers to hard and soft surfaces and includes, but are not limited to, tile grout, plaster, drywall, ceramic, cement, clay, bricks, stucco, plastic, wallpaper, fabric, tiles, cement, and vinyl flooring, heating and/or cooling fins, filters, vanes, baffles, vents, crevices in walls or ceilings, paper and wood products such as lumber, paper, and cardboard, woven products such as blankets, clothing, carpets, drapery and the like. The term surface also includes human surfaces, animal surfaces, military equipment, transportation equipment, children's items, plant surfaces, seeds, outdoor surfaces, soft surfaces, air, wounds, and medical instruments, and the like.

As used herein “pouch” refers to a hollow receptacle defining a volume. The pouch is “closed” in the sense that the actives are substantially retained within the pouch and the pouch volume is substantially sealed around its perimeter. However, the material or materials used to construct the pouch are chosen to allow exit of the gas generated. A pouch can be a sachet, an envelope or a receptacle defining an enclosed surface. The pouch can wholly be constructed from gas permeable layers, or the gas permeable layer can comprise only a portion, e.g. one side of a pouch. The remainder of the pouch can include impermeable materials or other materials.

As used herein the term “sachet” means a closed receptacle for actives. The sachet is “closed” in the sense that the reactants are substantially retained within the sachet and the sachet volume is substantially sealed around its perimeter. However, the material or materials used to construct the sachet are chosen to allow exit of the gas generated. The material or materials used to construct sachets are referred to herein as “sachet layers.” Sachet layers typically are constructed from a planar material, such as, but not limited to, a polymeric sheet or film. Preferred materials for sachet layers are described in greater detail below. Sachets can include more than one material, e.g. a sachet can comprise a barrier layer and sachet layer sealed about the perimeters of the layers to define a closed receptacle for actives. Another example of a sachet is a rigid frame defining one or more openings and one or more layers, including at least one sachet layer, disposed about the one or more openings to define a closed receptacle for actives.

“Permeable layer,” as used herein, refers to a layer that permits passage of gas or vapor generated by an apparatus or other source of the present invention. Permeable layers typically are constructed from polymeric materials. “Impermeable layer”, as used herein, refers to a layer that substantially prevents or hinders passage of the generated gas or vapor. Impermeable layers can be constructed from various materials, including polymeric material, glass, metal, metallized polymeric material and/or coated papers. As used herein, barrier layers are impermeable layers. The skilled artisan will appreciate that what is considered to be an “impermeable layer” and what is considered to be a “permeable layer” is defined relative to the transmission rates of the respective layers used to construct apparatus of the present invention and the desired gas emission characteristics or shelf life of the product. Relying upon the teachings disclosed herein, and the general knowledge in the art, the practitioner of ordinary skill will require only routine experimentation to identify and/or construct one or more impermeable layers and one or more permeable layers adapted for the purpose at hand.

“Selective transmission films” are films that are neither perforated nor porous, but instead transfer gases through the polymer structure of the film. Selective transmission films can be multilayered or mixed polymer materials, where the layers and the polymers are chosen for controlled transmission of gases, such as carbon dioxide and oxygen. Selective transmission films are preferred in dry applications because they allow the gas to diffuse out of the apparatus. Further, such layers also can be employed to retain the initiating agent once released from a frangible pouch. Moreover, the selective transmission film can increase the stability of the apparatus prior to its use because it may not readily allow ambient water to diffuse into the apparatus, which could prematurely initiate the reactants.

As used herein “water vapor selective” refers to a material that selectively allows permeation of water vapor and substantially impedes permeation of liquid water. Suitably, the material excludes permeation of liquid water. Typically, the water vapor selective material is hydrophobic. The skilled practitioner typically refers to water vapor selective material as water impermeable.

### Hypohalous Acid and Salts

In one embodiment, the compositions comprise hypohalite, defined as hypohalous acid and/or salts thereof. Suitable hypohalous acids and salts may be provided by a variety of sources, including compositions that lead to the formation of positive halide ions and/or hypohalite ions, as well as compositions that are organic based sources of halides, such as chloroisocyanurates, haloamines, haloimines, haloimides and haloamides, or mixtures thereof. These compositions may also produce hypohalous acid or hypohalite species in situ. Suitable hypohalous acids and salts for use herein include the alkali metal and alkaline earth metal hypochlorites, hypobromites, hypoiodites, chlorinated trisodium phosphate dodecahydrates, potassium and sodium dichloroisocyanurates, potassium and sodium trichlorocyanurates, N-chloroimides, N-chloroamides, N-chlorosulfamide, N-chloroamines, chlorohydantoins such as dichlorodimethyl hydantoin and chlorobromo dimethylhydantoin, bromo-compounds corresponding to the chloro-compounds above, and compositions which generate the corresponding hypohalous acids, or mixtures thereof.

In one embodiment wherein the compositions herein are liquid, said hypohalite compositions is an alkali metal and/or alkaline earth metal hypochlorite, or mixtures thereof. Compositions may be an alkali metal and/or alkaline earth metal hypochlorite selected from the group consisting of sodium hypochlorite, potassium hypochlorite, magnesium hypochlorite, lithium hypochlorite and calcium hypochlorite, and mixtures thereof.

The hypohalous acids and salt composition may be an equilibrium mixture of hypochlorous acid and sodium hypochlorite. The oxidant active species is present in an amount from above zero to about 15 weight percent of the composition, or from about 0.001 weight percent (10 ppm) to about 10 weight percent of the composition, or from about 0.001 weight percent (10 ppm) to about 1 weight percent of the composition, or from about 0.005 (50 ppm) to about 0.05 weight percent of the composition.

Other oxidants are also possible including peroxygen compounds such as hydrogen peroxide and other oxidants such as 5 weight percent of the composition. The compositions may have between 40 ppm to about 600 ppm available chlorine, or between 40 ppm to about 500 ppm available chlorine, or between 40 ppm to about 400 ppm available chlorine, or between 40 ppm and 1200 ppm, or from 40 ppm to less than 200 ppm, or from 40 ppm to less than 100 ppm, or between 50 ppm to about 400 ppm available chlorine dioxide. In some embodiments the oxidant or oxidants are effective against mold, mildew, odors, allergens, biofilm, etc. in the absence of any other antimicrobial agent or active ingredient, such as metal ions, quaternary ammonium compounds, or volatile alcohols.

The amount of available halogen oxidant in the composition is determined by placing samples of the composition into about 50 milliliters of distilled water, followed by addition of about 10 milliliters of a 10 weight/weight percent solution of potassium iodide and addition of about 10 milliliters of a 10 volume percent solution of sulfuric acid, the resulting mixture being well stirred. The resulting yellow to brown solution, whose color is the result of oxidation of free iodine ion (I⁻) to molecular iodine (I₂), was then volumetrically titrated to an essentially colorless endpoint by addition of standardized 0.1 Molar sodium thiosulfate (Na₂S₂O₃) titrant. Calculation then expresses the result as percent of available molecular chlorine (Cl₂), that is to say assigning two equivalents per mole of titrated hypohalite oxidant. Stability results are then expressed by repeated assays over time using identically prepared samples resulting from the same composition, normalized to 100 percent representative of the starting available chlorine measured initially.

During the course of evaluating various oxidants and antimicrobials for their allergen deactivating ability, we have found that a very dilute solution (on the order of 40-80 ppm) of primarily hypochlorous acid can effectively deactivate allergens. Presumably the low levels of oxidant are still able to break up the allergen proteins, rendering them biologically inert.

Additional descriptions of dilute hypochlorite and packaging technology are found in Co-pending U.S. Pat. App. 2005/0232848, entitled “Packaging for Dilute Hypochlorite”; Co-pending U.S. Pat. App. 2005/0221113, entitled “Packaging for Dilute Hypochlorite”; Co-pending Application U.S. Pat. App. 2005/0232847, entitled “Method for Diluting Hypochlorite”; and Co-pending Application U.S. Pat. App. 2005/0214386, entitled “Methods for deactivating allergens and preventing disease”, and all or which are incorporated herein.

The anodic oxidation of chloride in an electrolysis cell results in the production of a number of oxychlorine ions including hypochlorite, chlorite, chlorate, and perchlorate. Chlorite is readily oxidized to chlorate. Perchlorate may be an undesirable contaminant in the environment due to its low reactivity, high mobility, and inhibition of thyroid function. The production of hypochlorite via chlorination of caustic water is not believed to result in the formation of perchlorate. This route may be advantageous for certain uses where minor amounts of perchlorate would be undesirable.

### Antimicrobial Actives and Registered Actives

In one embodiment the active is an antimicrobial active. In one embodiment the active is sufficient to satisfy the requirements for US EPA registration as a sanitizer or disinfectant. Certain chemical compositions for disinfecting, sanitizing, and deodorizing, including acidic materials, antibacterial materials, and solvents that kill bacteria require EPA registration as a pesticide for health concerns. The requirements for different surfaces and target areas are different. Thus, an active registered to sanitize a hard surface may not be effective or registered to sanitize a soft surface.

### Other Antimicrobial Actives

Suitable antimicrobial agents include quaternary ammonium compounds. Non-limiting examples of these quaternary compounds include benzalkonium chlorides and/or substituted benzalkonium chlorides, di(C₆-C₁₄)alkyl di short chain (C₁₋₄ alkyl and/or hydroxyalkl) quaternaryammonium salts, N-(3-chloroallyl) hexaminium chlorides, benzethonium chloride, methylbenzethonium chloride, and cetylpyridinium chloride. Other quaternary compounds include the group consisting of dialkyldimethyl ammonium chlorides, alkyl dimethylbenzylammonium chlorides, dialkylmethylbenzylammonium chlorides, and mixtures thereof. Biguanide antimicrobial actives including, but not limited to polyhexamethylene biguanide hydrochloride, p-chlorophenyl biguanide; 4-chlorobenzhydryl biguanide, halogenated hexidine such as, but not limited to, chlorhexidine (1,1′-hexamethylene-bis-5-(4-chlorophenyl biguanide) and its salts are also in this class. There are three principal suppliers of quaternary based antimicrobials that are registered as actives for this type of use with the EPA. These companies are Lonza, Stepan and Mason Chemical Company. The trade names under which they are marketed are Bardac, BTC and Maquat respectively.

Suitable antibacterial metal salts include salts of metals in groups 3b-7b,8 and 3a-5a. Specifically are the salts of aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof. Suitable metallic antimicrobials include silver compounds as described in U.S. Pat. No. 6,180,584 to Sawan.

Suitable phenolic antimicrobials include o-penyl-phenol, o-benzyl(p-chlorophenol), 4-tertamylphenol and mixtures thereof.

Suitable essential oil antimicrobials include those essential oils which exhibit anti-microbial activity. By “actives of essential oils”, it is meant herein any ingredient of essential oils that exhibit anti-microbial activity. It is speculated that said anti-microbial essential oils and actives thereof act as proteins denaturing agents. Such anti-microbial essential oils include, but are not limited to, those obtained from thyme, lemongrass, citrus, lemons, orange, anise, clove, aniseed, pine, cinnamon, geranium, roses, mint, lavender, citronella, eucalyptus, peppermint, camphor, ajowan, sandalwood, rosmarin, vervain, fleagrass, lemongrass, ratanhiae, cedar and mixtures thereof. Suitable anti-microbial essential oils to be used herein are thyme oil, clove oil, cinnamon oil, geranium oil, eucalyptus oil, peppermint oil, citronella oil, ajowan oil, mint oil or mixtures thereof. Actives of essential oils to be used herein include, but are not limited to, thymol (present for example in thyme, ajowan), eugenol (present for example in cinnamon and clove), menthol (present for example in mint), geraniol (present for example in geranium and rose, citronella), verbenone (present for example in vervain), eucalyptol and pinocarvone (present in eucalyptus), cedrol (present for example in cedar), anethol (present for example in anise), carvacrol, hinokitiol, berberine, ferulic acid, cinnamic acid, methyl salicylic acid, methyl salycilate, terpineol, limonene and mixtures thereof. Suitable actives of essential oils to be used herein are thymol, eugenol, verbenone, eucalyptol, terpineol, cinnamic acid, methyl salicylic acid, limonene, geraniol or mixtures thereof.

Suitable oxidant antimicrobials include hydrogen peroxide and other peroxides, sources of hydrogen peroxide and other peroxides, generators of hydroxyl radical, peracid bleaches and peracid bleach precursors, as described in U.S. Pat. No. 6,548,467 to Baker et al. and U.S. Pat. No. 6,627,590 to Sherry et al.

Suitable acid antimicrobials include: citric acid, cresylic acid, dodecylbenzene sulfonic acid, phosphoric acid, salicylic acid, sorbic acid, sulfamic acid, acetic acid, benzoic acid, boric acid, capric acid, caproic acid, cyanuric acid, dihydroacetic acid, dimethylsulfamic acid, propionic acid, polyacrylic acid, 2-ethyl-hexanoic acid, formic acid, fumaric acid, 1-glutamic acid, isopropyl sulfamic acid, naphthenic acid, oxalic acid, phosphorus acid, valeric acid, benzene sulfonic acid, xylene sulfonic acid, as well as any acid listed as a registered pesticide active ingredient with the United States Environmental Protection Agency. Further useful acids include: sulfonic acids, maleic acid, acetic acid, adipic acid, lactic acid, butyric acid, gluconic acid, malic acid, tartaric acid, as well as glycolic acid. Desirably glycolic acid and citric acid are used as they are effective and in plentiful supply.

Antimicrobial agents are present, suitably at levels below about 0.5%, or below about 0.4%, or below 0.1%.

### Other Product Components

Other suitable components in any suitable amount may be used. Suitable ingredients include, but are not limited to: aesthetic agents, anti-filming agents, antiredopsition agents, anti-spotting agents, beads, binders, bleach activators, bleach catalysts, bleach stabilizing systems, bleaching agents, brighteners, buffering agents, builders, carriers, chelants, clay, color speckles, control release agents, corrosion inhibitors, dishcare agents, disinfectant, dispersant agents, dispersant polymers, draining promoting agents, drying agents, dyes, dye transfer inhibiting agents, enzymes, enzyme stabilizing systems, fillers, free radical inhibitors, fungicides, germicides, hydrotropes, opacifiers, perfumes, pH adjusting agents, pigments, processing aids, silicates, soil release agents, suds suppressors, surfactants, stabilizers, thickeners, zeolite, and mixtures thereof.

### Surfactants

The composition of the invention may contain surfactants either separate from the dilute hypohalous acid and salt or in the same composition. The surfactants should be stable to hypohalous acid or hypohalous acid salt if long term storage together is desired. If the solutions of the composition are generated prior to use, then surfactants having less stability may be used unless they are physically isolated. Examples of surfactants having relatively good stability can be found in U.S. Pat. Nos. 6,413,925 and 5,851,421. In general, surfactants such as amine oxide, alkylpolyglycoside, aryl sulfonates, quaternary ammonium compounds, are not compatible with dilute hypochlorite compositions for long term stability, especially dilute hypochlorite compositions of near neutral pH. The compositions may not have any surfactants for maximum stability.

The composition may contain one or more surfactants selected from anionic, nonionic, cationic, ampholytic, amphoteric and zwitterionic surfactants and mixtures thereof. A typical listing of anionic, nonionic, ampholytic, and zwitterionic classes, and species of these surfactants, is given in U.S. Pat. No. 3,929,678 to Laughlin and Heuring. A list of suitable cationic surfactants is given in U.S. Pat. No. 4,259,217 to Murphy. Where present, ampholytic, amphoteric and zwitteronic surfactants are generally used in combination with one or more anionic and/or nonionic surfactants. The surfactants may be present at a level of from about 0% to 90%, or from about 0.001% to 50%, or from 0.001% to 1.0%, or from about 0.01% to 25% by weight.

The composition may comprise an anionic surfactant. Essentially any anionic surfactants useful for detersive purposes can be comprised in the cleaning composition. These can include salts (including, for example, sodium, potassium, ammonium, and substituted ammonium salts such as mono-, di- and tri-ethanolamine salts) of the anionic sulfate, sulfonate, carboxylate and sarcosinate surfactants. Anionic surfactants may comprise a sulfonate or a sulfate surfactant. Anionic surfactants may comprise an alkyl sulfate, a linear or branched alkyl benzene sulfonate, or an alkyldiphenyloxide disulfonate, as described herein.

Other anionic surfactants include the isethionates such as the acyl isethionates, N-acyl taurates, fatty acid amides of methyl tauride, alkyl succinates and sulfosuccinates, monoesters of sulfosuccinate (for instance, saturated and unsaturated C12-C18 monoesters) diesters of sulfosuccinate (for instance saturated and unsaturated C6-C14 diesters), N-acyl sarcosinates. Resin acids and hydrogenated resin acids are also suitable, such as rosin, hydrogenated rosin, and resin acids and hydrogenated resin acids present in or derived from tallow oil. Anionic sulfate surfactants suitable for use herein include the linear and branched primary and secondary alkyl sulfates, alkyl ethoxysulfates, fatty oleoyl glycerol sulfates, alkyl phenol ethylene oxide ether sulfates, the C5-C17 acyl-N—(C1-C4 alkyl) and —N—(C1-C2 hydroxyalkyl) glucamine sulfates, and sulfates of alkylpolysacchanides such as the sulfates of alkylpolyglucoside (the nonionic nonsulfated compounds being described herein). Alkyl sulfate surfactants may be selected from the linear and branched primary C10-C18 alkyl sulfates, the C11-C15 branched chain alkyl sulfates, or the C12-C14 linear chain alkyl sulfates.

Alkyl ethoxysulfate surfactants may be selected from the group consisting of the C10-C18 alkyl sulfates which have been ethoxylated with from 0.5 to 20 moles of ethylene oxide per molecule. The alkyl ethoxysulfate surfactant may be a C11-C18, or a C11-C15 alkyl sulfate which has been ethoxylated with from 0.5 to 7, or from 1 to 5, moles of ethylene oxide per molecule. One aspect of the invention employs mixtures of the alkyl sulfate and/or sulfonate and alkyl ethoxysulfate surfactants. Such mixtures have been disclosed in PCT Patent App. No. WO 93/18124.

Anionic sulfonate surfactants suitable for use herein include the salts of C5-C20 linear alkylbenzene sulfonates, alkyl ester sulfonates, C6-C22 primary or secondary alkane sulfonates, C6-C24 olefin sulfonates, sulfonated polycarboxylic acids, alkyl glycerol sulfonates, fatty acyl glycerol sulfonates, fatty oleyl glycerol sulfonates, and any mixtures thereof. Suitable anionic carboxylate surfactants include the alkyl ethoxy carboxylates, the alkyl polyethoxy polycarboxylate surfactants and the soaps (‘alkyl carboxyls’), especially certain secondary soaps as described herein. Suitable alkyl ethoxy carboxylates include those with the formula RO(CH₂CH₂O)ₓCH₂COO⁻M⁺ wherein R is a C6 to C18 alkyl group, x ranges from 0 to 10, and the ethoxylate distribution is such that, on a weight basis, the amount of material where x is 0 is less than 20% and M is a cation. Suitable alkyl polyethoxypolycarboxylate surfactants include those having the formula RO—(CHR¹—CHR²—O)—R³ wherein R is a C6 to C18 alkyl group, x is from 1 to 25, R¹ and R² are selected from the group consisting of hydrogen, methyl acid radical, succinic acid radical, hydroxysuccinic acid radical, and mixtures thereof, and R³ is selected from the group consisting of hydrogen, substituted or unsubstituted hydrocarbon having between 1 and 8 carbon atoms, and mixtures thereof.

Suitable soap surfactants include the linear saturated soaps, such as lauric acid. Also suitable are secondary soap surfactants, which contain a carboxyl unit connected to a secondary carbon. Suitable secondary soap surfactants for use herein are water-soluble members selected from the group consisting of the water-soluble salts of 2-methyl-1-undecanoic acid, 2-ethyl-1-decanoic acid, 2-propyl-1-nonanoic acid, 2-butyl-1-octanoic acid and 2-pentyl-1-heptanoic acid. Certain soaps may also be included as suds suppressors.

Other suitable anionic surfactants are the alkali metal sarcosinates of formula R—CON(R¹) CH—)COOM, wherein R is a C5-C17 linear or branched alkyl or alkenyl group, R¹ is a C1-C4 alkyl group and M is an alkali metal ion. Examples are the myristyl and oleoyl methyl sarcosinates in the form of their sodium salts.

Essentially any alkoxylated nonionic surfactants are suitable herein, for instance, ethoxylated and propoxylated nonionic surfactants. Nonionic surfactants with stability to hypohalous acid or hypohalous acid salt, such as capped nonionics, are especially suitable. Alkoxylated surfactants can be selected from the classes of the nonionic condensates of alkyl phenols, nonionic ethoxylated alcohols, nonionic ethoxylated/propoxylated fatty alcohols, nonionic ethoxylate/propoxylate condensates with propylene glycol, and the nonionic ethoxylate condensation products with propylene oxide/ethylene diamine adducts.

The condensation products of aliphatic alcohols with from 1 to 25 moles of alkylene oxide, particularly ethylene oxide and/or propylene oxide, are suitable for use herein. The alkyl chain of the aliphatic alcohol can either be straight or branched, primary or secondary, and generally contains from 6 to 22 carbon atoms. Also suitable are the condensation products of alcohols having an alkyl group containing from 8 to 20 carbon atoms with from 2 to 10 moles of ethylene oxide per mole of alcohol.

Polyhydroxy fatty acid amides suitable for use herein are those having the structural formula R²CONR¹Z wherein: R¹ is H, C1-C4 hydrocarbyl, 2-hydroxyethyl, 2-hydroxypropyl, ethoxy, propoxy, or a mixture thereof, for instance, C1-C4 alkyl, or C1 or C2 alkyl; and R² is a C5-C31 hydrocarbyl, for instance, straight-chain C5-C19 alkyl or alkenyl, or straight-chain C9-C17 alkyl or alkenyl, or straight-chain C11-C17 alkyl or alkenyl, or mixture thereof-, and Z is a polyhydroxyhydrocarbyl having a linear hydrocarbyl chain with at least 3 hydroxyls directly connected to the chain, or an alkoxylated derivative (for example, ethoxylated or propoxylated) thereof. Z may be derived from a reducing sugar in a reductive amination reaction, for example, when Z is a glycityl.

Suitable fatty acid amide surfactants include those having the formula: R¹CON(R²)₂ wherein R¹ is an alkyl group containing from 7 to 21, or from 9 to 17 carbon atoms and each R² is selected from the group consisting of hydrogen, C1-C4 alkyl, C1-C4 hydroxyalkyl, and —(C₂H₄O)ₓH, where x is in the range of from 1 to 3.

Suitable alkylpolysaccharides for use herein are disclosed in U.S. Pat. No. 4,565,647 to Llenado, having a hydrophobic group containing from 6 to 30 carbon atoms and a polysaccharide, e.g., a polyglycoside, hydrophilic group containing from 1.3 to 10 saccharide units. Alkylpolyglycosides may have the formula: R²O(CₙH₂ₙO)ₜ(glycosyl)ₓ wherein R² is selected from the group consisting of alkyl, alkylphenyl, hydroxyalkyl, hydroxyalkylphenyl, and mixtures thereof in which the alkyl groups contain from 10 to 18 carbon atoms; n is 2 or 3; t is from 0 to 10, and x is from 1.3 to 8. The glycosyl may be derived from glucose.

Suitable amphoteric surfactants for use herein include the amine oxide surfactants and the alkyl amphocarboxylic acids. Suitable amine oxides include those compounds having the formula R³(OR⁴)_{X}NO(R⁵)₂ wherein R³ is selected from an alkyl, hydroxyalkyl, acylamidopropyl and alkylphenyl group, or mixtures thereof, containing from 8 to 26 carbon atoms; R⁴ is an alkylene or hydroxyalkylene group containing from 2 to 3 carbon atoms, or mixtures thereof, x is from 0 to 5, preferably from 0 to 3; and each R⁵ is an alkyl or hydroxyalkyl group containing from 1 to 3, or a polyethylene oxide group containing from 1 to 3 ethylene oxide groups. Suitable amine oxides are C10-C18 alkyl dimethylamine oxide, and C10-18 acylamido alkyl dimethylamine oxide. A suitable example of an alkyl amphodicarboxylic acid is Miranol™ C2M Conc. manufactured by Miranol, Inc., Dayton, N.J.

Zwitterionic surfactants can also be incorporated into the cleaning compositions. These surfactants can be broadly described as derivatives of secondary and tertiary amines, derivatives of heterocyclic secondary and tertiary amines, or derivatives of quaternary ammonium, quaternary phosphonium or tertiary sulfonium compounds. Betaine and sultaine surfactants are exemplary zwittenionic surfactants for use herein.

Suitable betaines are those compounds having the formula R(R¹)₂N⁺R²COO⁻ wherein R is a C6-C18 hydrocarbyl group, each R¹ is typically C1-C3 alkyl, and R² is a C1-C5 hydrocarbyl group. Suitable betaines are C12-18 dimethyl-ammonio hexanoate and the C10-18 acylamidopropane (or ethane) dimethyl (or diethyl) betaines. Complex betaine surfactants are also suitable for use herein.

Suitable cationic surfactants to be used herein include the quaternary ammonium surfactants. The quaternary ammonium surfactant may be a mono C6-C16, or a C6-C10 N-alkyl or alkenyl ammonium surfactant wherein the remaining N positions are substituted by methyl, hydroxyethyl or hydroxypropyl groups. Suitable are also the mono-alkoxylated and bis-alkoxylated amine surfactants.

Another suitable group of cationic surfactants, which can be used in the cleaning compositions, are cationic ester surfactants. The cationic ester surfactant is a compound having surfactant properties comprising at least one ester (i.e. —COO—) linkage and at least one cationically charged group. Suitable cationic ester surfactants, including choline ester surfactants, have for example been disclosed in U.S. Pat. Nos. 4,228,042, 4,239,660 and 4,260,529. The ester linkage and cationically charged group may be separated from each other in the surfactant molecule by a spacer group consisting of a chain comprising at least three atoms (i.e. of three atoms chain length), or from three to eight atoms, or from three to five atoms, or three atoms. The atoms forming the spacer group chain are selected from the group consisting, of carbon, nitrogen and oxygen atoms and any mixtures thereof, with the proviso that any nitrogen or oxygen atom in said chain connects only with carbon atoms in the chain. Thus spacer groups having, for example, —O—O— (i.e. peroxide), —N—N—, and —N—O— linkages are excluded, whilst spacer groups having, for example —CH₂—O—, CH₂— and —CH₂—NH—CH₂— linkages are included. The spacer group chain may comprise only carbon atoms, or the chain is a hydrocarbyl chain.

The composition may comprise cationic mono-alkoxylated amine surfactants, for instance, of the general formula: R¹R²R³N⁺ApR⁴X⁻ wherein R¹ is an alkyl or alkenyl moiety containing from about 6 to about 18 carbon atoms, or from 6 to about 16 carbon atoms, or from about 6 to about 14 carbon atoms; R² and R³ are each independently alkyl groups containing from one to about three carbon atoms, for instance, methyl, for instance, both R² and R³ are methyl groups; R⁴ is selected from hydrogen, methyl and ethyl; X⁻ is an anion such as chloride, bromide, methylsulfate, sulfate, or the like, to provide electrical neutrality; A is a alkoxy group, especially a ethoxy, propoxy or butoxy group; and p is from 0 to about 30, or from 2 to about 15, or from 2 to about 8. The ApR⁴ group in the formula may have p=1 and is a hydroxyalkyl group, having no greater than 6 carbon atoms whereby the —OH group is separated from the quaternary ammonium nitrogen atom by no more than 3 carbon atoms. Suitable ApR⁴ groups are —CH₂CH₂—OH, —CH₂CH₂CH₂—OH, —CH₂CH(CH₃)—OH and —CH(CH₃)CH₂—OH. Suitable R¹ groups are linear alkyl groups, for instance, linear R¹ groups having from 8 to 14 carbon atoms.

Suitable cationic mono-alkoxylated amine surfactants for use herein are of the formula R¹(CH₃)(CH₃)N⁺(CH₂CH₂O)₂₋₅H X⁻ wherein R¹ is C10-C18 hydrocarbyl and mixtures thereof, especially C10-C14 alkyl, or C₁₀ and C12 alkyl, and X is any convenient anion to provide charge balance, for instance, chloride or bromide.

As noted, compounds of the foregoing type include those wherein the ethoxy (CH₂CH₂O) units (EO) are replaced by butoxy, isopropoxy [CH(CH₃)CH₂O] and [CH₂CH(CH₃)O] units (i-Pr) or n-propoxy units (Pr), or mixtures of EO and/or Pr and/or i-Pr units.

The cationic bis-alkoxylated amine surfactant may have the general formula: R¹R²N⁺ApR³A′qR⁴X⁻ wherein R¹ is an alkyl or alkenyl moiety containing from about 8 to about 18 carbon atoms, or from 10 to about 16 carbon atoms, or from about 10 to about 14 carbon atoms; R² is an alkyl group containing from one to three carbon atoms, for instance, methyl; R³ and R⁴ can vary independently and are selected from hydrogen, methyl and ethyl, X⁻ is an anion such as chloride, bromide, methylsulfate, sulfate, or the like, sufficient to provide electrical neutrality. A and A′ can vary independently and are each selected from C1-C4 alkoxy, for instance, ethoxy, (i.e., —CH₂CH₂O—), propoxy, butoxy and mixtures thereof, p is from 1 to about 30, or from 1 to about 4 and q is from 1 to about 30, or from 1 to about 4, or both p and q are 1.

Suitable cationic bis-alkoxylated amine surfactants for use herein are of the formula R¹CH₃N⁺(CH₂CH₂OH)(CH₂CH₂OH)X⁻, wherein R¹ is C10-C18 hydrocarbyl and mixtures thereof, or C10, C12, C14 alkyl and mixtures thereof, X⁻ is any convenient anion to provide charge balance, for example, chloride. With reference to the general cationic bis-alkoxylated amine structure noted above, since in one example compound R¹ is derived from (coconut) C12-C14 alkyl fraction fatty acids, R² is methyl and ApR³ and A′qR⁴ are each monoethoxy.

Other cationic bis-alkoxylated amine surfactants useful herein include compounds of the formula: R¹R²N—(CH₂CH₂O)ₚH—(CH₂CH₂O)_{q}H X⁻ wherein R¹ is C10-C18 hydrocarbyl, or C10-C14 alkyl, independently p is 1 to about 3 and q is 1 to about 3, R² is C1-C3 alkyl, for example, methyl, and X⁻ is an anion, for example, chloride or bromide.

Other compounds of the foregoing type include those wherein the ethoxy (CH₂CH₂O) units (EO) are replaced by butoxy (Bu) isopropoxy [CH(CH₃)CH₂O] and [CH₂CH(CH₃)O] units (i-Pr) or n-propoxy units (Pr), or mixtures of EO and/or Pr and/or i-Pr units.

The inventive compositions may include at least one fluorosurfactant selected from nonionic fluorosurfactants, cationic fluorosurfactants, and mixtures thereof which are soluble or dispersible in the aqueous compositions being taught herein, sometimes compositions which do not include further detersive surfactants, or further organic solvents, or both. Suitable nonionic fluorosurfactant compounds are found among the materials presently commercially marketed under the tradename Fluorad® (ex. 3M Corp.) Exemplary fluorosurfactants include those sold as Fluorad® FC-740, generally described to be fluorinated alkyl esters; Fluorad® FC-430, generally described to be fluorinated alkyl esters; Fluorad® FC-431, generally described to be fluorinated alkyl esters; and, Fluorad® FC-170-C, which is generally described as being fluorinated alkyl polyoxyethylene ethanols.

Suitable nonionic fluorosurfactant compounds include those which is believed to conform to the following formulation:

CₙF₂ₙ₊₁SO₂N(C₂H₅)(CH₂CH₂O)ₓCH₃ wherein: n has a value of from 1-12, or from 4-12, or 8; x has a value of from 4-18, or from 4-10, or 7; which is described to be a nonionic fluorinated alkyl alkoxylate and which is sold as Fluorad® FC-171 (ex. 3M Corp., formerly Minnesota Mining and Manufacturing Co.).

Additionally suitable nonionic fluorosurfactant compounds are also found among the materials marketed under the tradename ZONYL® (DuPont Performance Chemicals). These include example, ZONYL® FSO and ZONYL® FSN. These compounds have the following formula: RfCH₂CH₂—O—(CH₂CH₂O)ₓH where Rf is F(CF₂CF₂)_{y}. For ZONYL® FSO, x is 0 to about 15 and y is 1 to about 7. For ZONYL® FSN, x is 0 to about 25 and y is 1 to about 9.

An example of a suitable cationic fluorosurfactant compound has the following structure: CₙF₂ₙ₊₁SO₂NHC₃H₆N⁺(CH₃)₃I⁻ where n˜8. This cationic fluorosurfactant is available under the tradename Fluorad® FC-135 from 3M. Another example of a suitable cationic fluorosurfactant is F₃—(CF₂)ₙ—(CH₂)ₘSCH₂CHOH—CH₂—N⁺R₁R₂R₃ Cl⁻ wherein: n is 5-9 and m is 2, and R₁, R₂ and R₃ are —CH₃. This cationic fluorosurfactant is available under the tradename ZONYL® FSD (available from DuPont, described as 2-hydroxy-3-((gamma-omega-perfluoro-C₆₋₂₀-alkyl)thio)-N,N,N-trimethyl-1-propyl ammonium chloride). Other cationic fluorosurfactants suitable for use in the present invention are also described in EP 866,115 to Leach and Niwata.

The fluorosurfactant selected from the group of nonionic fluorosurfactant, cationic fluorosurfactant, and mixtures thereof may be present in amounts of from 0.001 to 5% wt., preferably from 0.01 to 1% wt., and more preferably from 0.01 to 0.5% wt.

### Solvent

The composition of the invention may contain solvents. The solvents should be stable to hypohalous acid or hypohalous acid salt if long term storage together is desired. However, even hypochlorite stable solvents are generally not stable in dilute hypochlorite compositions, such as those containing 40 to 200 ppm at near neutral pH. The compositions may not have any solvents, aside from water, for maximum stability. Suitable solvents might be hydrocarbons or esters not having any alcohol or olefinic groups. If the solutions of the composition are generated prior to or during use, then solvents having less stability may be used.

Suitable organic solvents include, but are not limited to, C₁₋₆ alkanols, C₁₋₆ diols, C₁₋₁₀ alkyl ethers of alkylene glycols, C₃₋₂₄ alkylene glycol ethers, polyalkylene glycols, short chain carboxylic acids, short chain esters, isoparafinic hydrocarbons, mineral spirits, alkylaromatics, terpenes, terpene derivatives, terpenoids, terpenoid derivatives, formaldehyde, and pyrrolidones. Alkanols include, but are not limited to, methanol, ethanol, n-propanol, isopropanol, butanol, pentanol, and hexanol, and isomers thereof. Diols include, but are not limited to, methylene, ethylene, propylene and butylene glycols. Alkylene glycol ethers include, but are not limited to, ethylene glycol monopropyl ether, ethylene glycol monobutyl ether, ethylene glycol monohexyl ether, diethylene glycol monopropyl ether, diethylene glycol monobutyl ether, diethylene glycol monohexyl ether, propylene glycol methyl ether, propylene glycol ethyl ether, propylene glycol n-propyl ether, propylene glycol monobutyl ether, propylene glycol t-butyl ether, di- or tri-polypropylene glycol methyl or ethyl or propyl or butyl ether, acetate and propionate esters of glycol ethers. Short chain carboxylic acids include, but are not limited to, acetic acid, glycolic acid, lactic acid and propionic acid. Short chain esters include, but are not limited to, glycol acetate, and cyclic or linear volatile methylsiloxanes. Water insoluble solvents such as isoparafinic hydrocarbons, mineral spirits, alkylaromatics, terpenoids, terpenoid derivatives, terpenes, and terpenes derivatives can be mixed with a water-soluble solvent when employed.

Examples of organic solvent having a vapor pressure less than 0.1 mm Hg (20° C.) include, but are not limited to, dipropylene glycol n-propyl ether, dipropylene glycol t-butyl ether, dipropylene glycol n-butyl ether, tripropylene glycol methyl ether, tripropylene glycol n-butyl ether, diethylene glycol propyl ether, diethylene glycol butyl ether, dipropylene glycol methyl ether acetate, diethylene glycol ethyl ether acetate, and diethylene glycol butyl ether acetate (all available from ARCO Chemical Company) or which are incorporated herein.

The solvents can be present at a level of from 0.001% to 10%, or from 0.01% to 10%, or from 1% to 4% by weight.

### Additional Adjuncts

The compositions optionally contain one or more of the following adjuncts: stain and vapor pressure modifiers, soil repellants, lubricants, odor control agents, perfumes, fragrances and fragrance release agents, brighteners, and fluorescent whitening agents. Other adjuncts include, but are not limited to, acids, electrolytes, dyes and/or colorants, solubilizing materials, stabilizers, thickeners, defoamers, hydrotropes, cloud point modifiers, preservatives, and other polymers. For maximum stability, the compositions can contain no carboxylic acids, no carboxylic acids with hydroxyl or olefinic groups, no alcohols, no amines such as primary or secondary amines, no fragrances, no colorants, no flavorants, no preservatives, no odor or taste masking agents, and low salt content, for example less than 0.3 g/L, or less than 0.2 g/L.

The solubilizing materials, when used, include, but are not limited to, hydrotropes (e.g. water soluble salts of low molecular weight organic acids such as the sodium and/or potassium salts of toluene, cumene, and xylene sulfonic acid). The acids, when used, include, but are not limited to, mineral acids, organic hydroxy acids, citric acids, keto acid, and the like. Electrolytes, when used, include, calcium, sodium and potassium chloride. Thickeners, when used, include, but are not limited to, polyacrylic acid, xanthan gum, calcium carbonate, aluminum oxide, alginates, guar gum, methyl, ethyl, clays, and/or propyl hydroxycelluloses. Defoamers, when used, include, but are not limited to, silicones, aminosilicones, silicone blends, and/or silicone/hydrocarbon blends.

Preservatives, when used, include, but are not limited to, mildewstat or bacteriostat, methyl, ethyl and propyl parabens, phosphates such as trisodium phosphate, short chain organic acids (e.g. acetic, lactic and/or glycolic acids), bisguanidine compounds (e.g. Dantagard® and/or Glydant®) and/or short chain alcohols (e.g. ethanol and/or IPA). The mildewstat or bacteriostat includes, but is not limited to, mildewstats (including non-isothiazolone compounds) including Kathon GC, a 5-chloro-2-methyl-4-isothiazolin-3-one, KATHON® ICP, a 2-methyl-4-isothiazolin-3-one, and a blend thereof, and KATHON® 886, a 5-chloro-2-methyl-4-isothiazolin-3-one, all available from Rohm and Haas Company; BRONOPOL®, a 2-bromo-2-nitropropane 1, 3 diol, from Boots Company Ltd., PROXEL® CRL, a propyl-p-hydroxybenzoate, from ICI PLC; NIPASOL® M, an o-phenyl-phenol, Na⁺ salt, from Nipa Laboratories Ltd., DOWICIDE® A, a 1,2-Benzoisothiazolin-3-one, from Dow Chemical Co., Nipacides from Clariant, and IRGASAN® DP 200, a 2,4,4′-trichloro-2-hydroxydiphenylether, from Ciba-Geigy A.G.

### Antimicrobial Agent

The composition of the invention may contain antimicrobial agents. The antimicrobial agents should be stable to hypohalous acid or hypohalous acid salt if long term storage is desired. If the solutions of the composition are generated prior to use, then antimicrobial agents having less stability may be used.

Antimicrobial agents include quaternary ammonium compounds and phenolics. Non-limiting examples of these quaternary compounds include benzalkonium chlorides and/or substituted benzalkonium chlorides, di(C6-C14)alkyl di short chain (C14 alkyl and/or hydroxyalkyl) quaternary ammonium salts, N-(3-chloroallyl) hexammonium chlorides, benzethonium chloride, methylbenzethonium chloride, and cetylpyridinium chloride. Other quaternary compounds include the group consisting of dialkyldimethyl ammonium chlorides, alkyl dimethylbenzyl ammonium chlorides, dialkylmethylbenzylammonium chlorides, and mixtures thereof. Biguanide antimicrobial actives include, but are not limited to polyhexamethylene biguanide hydrochloride, p-chlorophenyl biguanide; 4-chlorobenzhydryl biguanide, halogenated hexidine such as, but not limited to, chlorhexidine (1,1′-hexamethylene-bis-5-(4-chlorophenyl biguanide) and its salts are also in this class.

### Builder/Buffer

The composition of the invention may contain a builder or buffer. The builder or buffer should be stable to hypohalous acid or hypohalous acid salt if long term storage is desired. If the solutions of the composition are generated prior to use, then builders or buffers having less stability may be used.

The composition may include a builder or buffer, which can be used as a pH adjusting agent or as a sequestering agent in the composition. A variety of builders or buffers can be used and they include, but are not limited to, phosphate-silicate compounds, carbon dioxide or carbonate, zeolites, alkali metal, ammonium and substituted ammonium polyacetates, trialkali salts of nitrilotriacetic acid, carboxylates, polycarboxylates, carbonates, bicarbonates, polyphosphates, aminopolycarboxylates, polyhydroxysulfonates, and starch derivatives.

Builders or buffers can also include polyacetates and polycarboxylates. The polyacetate and polycarboxylate compounds include, but are not limited to, sodium, potassium, lithium, ammonium, and substituted ammonium salts of ethylenediamine tetraacetic acid, ethylenediamine triacetic acid, ethylenediamine tetrapropionic acid, diethylenetriamine pentaacetic acid, nitrilotriacetic acid, oxydisuccinic acid, iminodisuccinic acid, mellitic acid, polyacrylic acid or polymethacrylic acid and copolymers, benzene polycarboxylic acids, gluconic acid, sulfamic acid, oxalic acid, phosphoric acid, phosphonic acid, organic phosphonic acids, acetic acid, and citric acid. These builders or buffers can also exist either partially or totally in the protonated or neutralized form.

The builder agent can include sodium and/or potassium salts of EDTA and substituted ammonium salts. The substituted ammonium salts include, but are not limited to, ammonium salts of methylamine, dimethylamine, butylamine, butylenediamine, propylamine, triethylamine, trimethylamine, monoethanolamine, diethanolamine, triethanolamine, isopropanolamine, ethylenediamine tetraacetic acid and propanolamine.

Buffering and pH adjusting agents, when used, include, but are not limited to, organic acids, mineral acids, alkali metal and alkaline earth salts of silicate, metasilicate, polysilicate, borate, hydroxide, carbonate, carbamate, phosphate, polyphosphate, pyrophosphates, triphosphates, tetraphosphates, ammonia, hydroxide, monoethanolamine, monopropanolamine, diethanolamine, dipropanolamine, triethanolamine, and 2-amino-2-methylpropanol. Preferred buffering agents for compositions of this invention are nitrogen-containing materials. Some examples are amino acids such as lysine or lower alcohol amines like mono-, di-, and tri-ethanolamine. Other preferred nitrogen-containing buffering agents are tri(hydroxymethyl) amino methane (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol, N,N′-tetra-methyl-1,3-diamino-2-propanol, N,N-bis(2-hydroxyethyl)glycine (bicine) and N-tris(hydroxymethyl)methyl glycine (tricine). Other suitable buffers include ammonium carbamate, citric acid, acetic acid. Mixtures of any of the above are also acceptable. Useful inorganic buffers/alkalinity sources include ammonia, the alkali metal carbonates and alkali metal phosphates, e.g., sodium carbonate, sodium polyphosphate. For additional buffers see WO 95/07971, which is incorporated herein by reference. Other preferred pH adjusting agents include sodium or potassium hydroxide.

When employed, the builder, buffer, or pH adjusting agent comprises at least about 0.001% and typically about 0.01-5% by weight of the cleaning composition. Preferably, the builder or buffer content is about 0.01-2%.

### Substances Generally Recognized as Safe

Compositions according to the invention may comprise substances generally recognized as safe (GRAS), including essential oils, oleoresins (solvent-free) and natural extractives (including distillates), and synthetic flavoring materials and adjuvants. Compositions may also comprise GRAS materials commonly found in cotton, cotton textiles, paper and paperboard stock dry food packaging materials (referred herein as substrates) that have been found to migrate to dry food and, by inference may migrate into the inventive compositions when these packaging materials are used as substrates for the inventive compositions.

The composition of the invention may contain GRAS materials. The GRAS materials should be stable to hypohalous acid or hypohalous acid salt if long term storage is desired. If the solutions of the composition are generated prior to use, then GRAS materials having less stability may be used.

Suitable GRAS materials are listed in the Code of Federal Regulations (CFR) Title 21 of the United States Food and Drug Administration, Department of Health and Human Services, Parts 180.20, 180.40 and 180.50, which are hereby incorporated by reference. These suitable GRAS materials include essential oils, oleoresins (solvent-free), and natural extractives (including distillates). The GRAS materials may be present in the compositions in amounts of up to about 10% by weight, preferably in amounts of 0.01 and 5% by weight.

Suitable GRAS materials include oils and oleoresins (solvent-free) and natural extractives (including distillates) derived from alfalfa, allspice, almond bitter (free from prussic acid), ambergris, ambrette seed, angelica, angostura (cusparia bark), anise, apricot kernel (persic oil), asafetida, balm (lemon balm), balsam (of Peru), basil, bay leave, bay (myrcia oil), bergamot (bergamot orange), bois de rose (Aniba rosaeodora Ducke), cacao, camomile (chamomile) flowers, cananga, capsicum, caraway, cardamom seed (cardamon), carob bean, carrot, cascarilla bark, cassia bark, Castoreum, celery seed, cheery (wild bark), chervil, cinnamon bark, Civet (zibeth, zibet, zibetum), ceylon (Cinnamomum zeylanicum Nees), cinnamon (bark and leaf), citronella, citrus peels, clary (clary sage), clover, coca (decocainized), coffee, cognac oil (white and green), cola nut (kola nut), coriander, cumin (cummin), curacao orange peel, cusparia bark, dandelion, dog grass (quackgrass, triticum), elder flowers, estragole (esdragol, esdragon, estragon, tarragon), fennel (sweet), fenugreek, galanga (galangal), geranium, ginger, grapefruit, guava, hickory bark, horehound (hoarhound), hops, horsemint, hyssop, immortelle (Helichrysum augustifolium DC), jasmine, juniper (berries), laurel berry and leaf, lavender, lemon, lemon grass, lemon peel, lime, linden flowers, locust bean, lupulin, mace, mandarin (Citrus reticulata Blanco), marjoram, mate, menthol (including menthyl acetate), molasses (extract), musk (Tonquin musk), mustard, naringin, neroli (bigarade), nutmeg, onion, orange (bitter, flowers, leaf, flowers, peel), origanum, palmarosa, paprika, parsley, peach kernel (persic oil, pepper (black, white), peanut (stearine), peppermint, Peruvian balsam, petitgrain lemon, petitgrain mandarin (or tangerine), pimenta, pimenta leaf, pipsissewa leaves, pomegranate, prickly ash bark, quince seed, rose (absolute, attar, buds, flowers, fruit, hip, leaf), rose geranium, rosemary, safron, sage, St. John's bread, savory, schinus molle (Schinus molle L), sloe berriers, spearmint, spike lavender, tamarind, tangerine, tarragon, tea (Thea sinensis L.), thyme, tuberose, turmeric, vanilla, violet (flowers, leaves), wild cherry bark, ylang-ylang and zedoary bark.

Suitable synthetic flavoring substances and adjuvants are listed in the Code of Federal Regulations (CFR) Title 21 of the United States Food and Drug Administration, Department of Health and Human Services, Part 180.60, which is hereby incorporated by reference. These GRAS materials may be present in the compositions in amounts of up to about 1% by weight, preferably in amounts of 0.01 and 0.5% by weight.

Suitable synthetic flavoring substances and adjuvants that are generally recognized as safe for their intended use, include acetaldehyde (ethanal), acetoin (acetyl methylcarbinol), anethole (parapropenyl anisole), benzaldehyde (benzoic aldehyde), n-Butyric acid (butanoic acid), d- or l-carvone (carvol), cinnamaldehyde (cinnamic aldehyde), citral (2,6-dimethyloctadien-2,6-al-8, gera-nial, neral), decanal (N-decylaldehyde, capraldehyde, capric aldehyde, caprinaldehyde, aldehyde C-10), ethyl acetate, ethyl butyrate, 3-Methyl-3-phenyl glycidic acid ethyl ester (ethyl-methyl-phenyl-glycidate, so-called strawberry aldehyde, C-16 aldehyde), ethyl vanillin, geraniol (3,7-dimethyl-2,6 and 3,6-octadien-1-ol), geranyl acetate (geraniol acetate), limonene (d-, l-, and dl-), linalool (linalol, 3,7-dimethyl-1,6-octadien-3-ol), linalyl acetate (bergamol), methyl anthranilate (methyl-2-aminobenzoate), piperonal (3,4-methylenedioxy-benzaldehyde, heliotropin) and vanillin.

Suitable GRAS substances that may be present in the inventive compositions that have been identified as possibly migrating to food from cotton, cotton textiles, paper and paperboard materials used in dry food packaging materials are listed in the Code of Federal Regulations (CFR) Title 21 of the United States Food and Drug Administration, Department of Health and Human Services, Parts 180.70 and 180.90, which are hereby incorporated by reference. The GRAS materials may be present in the compositions either by addition or incidentally owing to migration from the substrates to the compositions employed in the invention, or present owing to both mechanisms. If present, the GRAS materials may be present in the compositions in amounts of up to about 1% by weight.

Suitable GRAS materials that are suitable for use in the invention, identified as originating from either cotton or cotton textile materials used as substrates in the invention, include beef tallow, carboxymethylcellulose, coconut oil (refined), cornstarch, gelatin, lard, lard oil, oleic acid, peanut oil, potato starch, sodium acetate, sodium chloride, sodium silicate, sodium tripolyphosphate, soybean oil (hydrogenated), talc, tallow (hydrogenated), tallow flakes, tapioca starch, tetrasodium pyrophosphate, wheat starch and zinc chloride.

Suitable GRAS materials that are suitable for use in the invention, identified as originating from either paper or paperboard stock materials used as substrates in the invention, include alum (double sulfate of aluminum and ammonium potassium, or sodium), aluminum hydroxide, aluminum oleate, aluminum palmitate, casein, cellulose acetate, cornstarch, diatomaceous earth filler, ethyl cellulose, ethyl vanillin, glycerin, oleic acid, potassium sorbate, silicon dioxides, sodium aluminate, sodium chloride, sodium hexametaphosphate, sodium hydrosulfite, sodium phosphoaluminate, sodium silicate, sodium sorbate, sodium tripolyphosphate, sorbitol, soy protein (isolated), starch (acid modified, pregelatinized and unmodified), talc, vanillin, zinc hydrosulfite and zinc sulfate.

### Fragrance

The composition of the invention may contain fragrance. The fragrance should be stable to hypohalous acid or hypohalous acid salt if long term storage is desired. If the solutions of the composition are generated prior to use, then fragrances having less stability may be used.

Compositions of the present invention may comprise from about 0.001% to about 5% by weight of the fragrance. Compositions of the present invention may comprise from about 0.005% to about 2.5% by weight of the fragrance. Compositions of the present invention may comprise from about 0.01% to about 1% by weight of the fragrance.

As used herein the term “fragrance” relates to the mixture of perfume raw materials that are used to impart an overall pleasant odor profile to a composition. As used herein the term “perfume raw material” relates to any chemical compound which is odiferous when in an un-entrapped state, for example in the case of pro-perfumes, the perfume component is considered, for the purposes of this invention, to be a perfume raw material, and the pro-chemistry anchor is considered to be the entrapment material. In addition “perfume raw materials” are defined by materials with a ClogP value preferably greater than about 0.1, more preferably greater than about 0.5, even more preferably greater than about 1.0. As used herein the term “ClogP” means the logarithm to base 10 of the octanol/water partition coefficient. This can be readily calculated from a program called “CLOGP” which is available from Daylight Chemical Information Systems Inc., Irvine Calif., U.S.A. Octanol/water partition coefficients are described in more detail in U.S. Pat. No. 5,578,563.

The individual perfume raw materials which comprise a known natural oil can be found by reference to Journals commonly used by those skilled in the art such as “Perfume and Flavourist” or “Journal of Essential Oil Research”. In addition some perfume raw materials are supplied by the fragrance houses as mixtures in the form of proprietary specialty accords. In order that fragrance oils can be developed with the appropriate character for the present invention the perfume raw materials have been classified based upon two key physical characteristics:

boiling point (BP) measured at 1 atmosphere pressure. The boiling point of many fragrance materials are given in Perfume and Flavor Chemicals (Aroma Chemicals), Steffen Arctander (1969). Perfume raw materials for use in the present invention are divided into volatile raw materials (which have a boiling point of less than, or equal to, about 250° C.) and residual raw materials (which have a boiling point of greater than about 250° C., preferably greater than about 275° C.). All perfume raw materials will preferably have boiling points (BP) of about 500° C. or lower.

odor detection threshold which is defined as the lowest vapour concentration of that material which can be olfactorily detected. The odor detection threshold and some odor detection threshold values are discussed in e.g., “Standardized Human Olfactory Thresholds”, M. Devos et al, IRL Press at Oxford University Press, 1990, and “Compilation of Odor and Taste Threshold Values Data”, F. A. Fazzalar, editor ASTM Data Series DS 48A, American Society for Testing and Materials, 1978, both of said publications being incorporated by reference. Perfume raw materials for use in the present invention can be classified as those with a low odor detection threshold of less than 50 parts per billion, preferably less than 10 parts per billion and those with a high odor detection threshold which are detectable at greater than 50 parts per billion (values as determined from the reference above).

Since, in general, perfume raw materials refer to a single individual compound, their physical properties (such ClogP, boiling point, odor detection threshold) can be found by referencing the texts cited above. In the case that the perfume raw material is a natural oil, which comprises a mixture of several compounds, the physical properties of the complete oil should be taken as the weighted average of the individual components. In the case that the perfume raw material is a proprietary specialty accord the physical properties should be obtain from the Supplier.

In general a broad range of suitable perfume raw materials can be found in U.S. Pat. Nos. 4,145,184, 4,209,417, 4,515,705, and 4,152,272. Non-limiting examples of perfume raw materials which are useful for blending to formulate fragrances for the present invention are given below. Any perfume raw materials, natural oils or proprietary specialty accords known to a person skilled in the art can be used within the present invention.

Volatile perfume raw materials useful in the present invention are selected from, but are not limited to, aldehydes with a relative molecular mass of less than or equal to about 200, esters with a relative molecular mass of less than or equal to about 225, terpenes with a relative molecular mass of less than or equal to about 200, alcohols with a relative molecular mass of less than or equal to about 200 ketones with a relative molecular mass of less than or equal to about 200, nitriles, pyrazines, and mixtures thereof.

Examples of volatile perfume raw materials having a boiling point of less than, or equal to, 250° C., with a low odor detection are selected from, but are not limited to, anethol, methyl heptine carbonate, ethyl aceto acetate, para cymene, nerol, decyl aldehyde, para cresol, methyl phenyl carbinyl acetate, ionone alpha, ionone beta, undecylenic aldehyde, undecyl aldehyde, 2,6-nonadienal, nonyl aldehyde, octyl aldehyde. Further examples of volatile perfume raw materials having a boiling point of less than, or equal to, 250° C., which are generally known to have a low odour detection threshold include, but are not limited to, phenyl acetaldehyde, anisic aldehyde, benzyl acetone, ethyl-2-methyl butyrate, damascenone, damascone alpha, damascone beta, flor acetate, frutene, fructone, herbavert, iso cyclo citral, methyl isobutenyl tetrahydro pyran, isopropyl quinoline, 2,6-nonadien-1-ol, 2-methoxy-3-(2-methylpropyl)-pyrazine, methyl octine carbonate, tridecene-2-nitrile, allyl amyl glycolate, cyclogalbanate, cyclal C, melonal, gamma nonalactone, c is 1,3-oxathiane-2-methyl-4-propyl.

Other volatile perfume raw materials having a boiling point of less than, or equal to, 250° C., which are useful in the present invention, which have a high odor detection threshold, are selected from, but are not limited to, benzaldehyde, benzyl acetate, camphor, carvone, borneol, bornyl acetate, decyl alcohol, eucalyptol, linalool, hexyl acetate, iso-amyl acetate, thymol, carvacrol, limonene, menthol, iso-amyl alcohol, phenyl ethyl alcohol, alpha pinene, alpha terpineol, citronellol, alpha thujone, benzyl alcohol, beta gamma hexenol, dimethyl benzyl carbinol, phenyl ethyl dimethyl carbinol, adoxal, allyl cyclohexane propionate, beta pinene, citral, citronellyl acetate, citronellal nitrile, dihydro myrcenol, geraniol, geranyl acetate, geranyl nitrile, hydroquinone dimethyl ether, hydroxycitronellal, linalyl acetate, phenyl acetaldehyde dimethyl acetal, phenyl propyl alcohol, prenyl acetate, triplal, tetrahydrolinalool, verdox, cis-3-hexenyl acetate.

Examples of residual “middle and base note” perfume raw materials having a boiling point of greater than 250° C., which have a low odor detection threshold are selected from, but are not limited to, ethyl methyl phenyl glycidate, ethyl vanillin, heliotropin, indol, methyl anthranilate, vanillin, amyl salicylate, coumarin. Further examples of residual perfume raw materials having a boiling point of greater than 250° C. which are generally known to have a low odor detection threshold include, but are not limited to, ambrox, bacdanol, benzyl salicylate, butyl anthranilate, cetalox, ebanol, cis-3-hexenyl salicylate, lilial, gamma undecalactone, gamma dodecalactone, gamma decalactone, calone, cymal, dihydro iso jasmonate, iso eugenol, lyral, methyl beta naphthyl ketone, beta naphthol methyl ether, para hydroxyl phenyl butanone, 8-cyclohexadecen-1-one, oxocyclohexadecen-2-one/habanolide, florhydral, intreleven aldehyde.

Other residual “middle and base note” perfume raw materials having a boiling point of greater than 250° C. which are useful in the present invention, but which have a high odor detection threshold, are selected from, but are not limited to, eugenol, amyl cinnamic aldehyde, hexyl cinnamic aldehyde, hexyl salicylate, methyl dihydro jasmonate, sandalore, veloutone, undecavertol, exaltolide/cyclopenta-decanolide, zingerone, methyl cedrylone, sandela, dimethyl benzyl carbinyl butyrate, dimethyl benzyl carbinyl isobutyrate, triethyl citrate, cashmeran, phenoxy ethyl isobutyrate, iso eugenol acetate, helional, iso E super, ionone gamma methyl, pentalide, galaxolide, phenoxy ethyl propionate.

The composition may include a builder or buffer, which can be used as a pH adjusting agent or as a sequestering agent in the composition. The builder, buffer, or pH adjusting agent may be an inorganic buffer. Examples of buffers or pH adjusting agents include a hydroxide of alkali metal, a hydroxide of alkaline earth metal, an inorganic acid or a salt thereof, sodium hydroxide, potassium hydroxide, calcium hydroxide, hydrochloric acid, sulfuric acid, sodium sulfate, sodium nitrate, sodium chloride, sodium carbonate, potassium hydrogen carbonate, sodium hydrogen carbonate, magnesium sulfate, magnesium nitrate, magnesium chloride, magnesium carbonate, sodium triphosphate, potassium triphosphate, disodium hydrogenphosphate, dipotassium hydrogenphosphate, sodium dihydrogenphosphate, potassium dihydrogenphosphate, and sodium polyphosphate.

When employed, the builder, buffer, or pH adjusting agent comprises at least about 0.001% and typically about 0.001-0.5% of the composition. Preferably, the builder or buffer content is about 0.001-0.2%.

### Water and pH

The water should be present at a level of less than about 99.999%. The water may be deionized, filtered to remove impurities including metals and organic carbon, purified by reverse osmosis, purified by distillation, or any combination thereof. Purified water may be prepared by a process selected from the group consisting of sodium cation exchange, hydrogen cation exchange, reverse osmosis, activated carbon treatment, UV light treatment, UVC, ozone treatment, chlorination, ultrafiltration, nanofiltration, electrodialysis, and a combination thereof. During preparation there may be a need for hygiene and segregation to prevent the introduction of compounds that are oxidized by hypochlorite since these become more important at low concentrations where the loss of a few ppm may be significant.

The composition may be adjusted for pH using a pH adjusting agent. Suitable pH adjusting agents include carbon dioxide, alkali metal carbonate, alkali metal bicarbonate, alkali metal silicates, alkali metal hydroxide, alkali phosphate salt, alkaline earth phosphate salt, alkali borate salt, hydrochloric acid, nitric acid, sulfuric acid, alkali metal hydrogen sulfate, acetic acid, vinegar from various sources, other carboxylic acids, polycarboxylates, organic sulfonic acids, sulfamic acid, amine, alkyl amine, dialkyl amine, and trialkyl amine. The composition may have a pH from 1 to 13. The composition may have a pH from 2 to 12. The composition may have a pH from 2 to 5. The composition may have a pH from 5 to less than 8. The composition may have a pH from between 4 and less than 8. The composition may have a pH between 6 to and less than 8. The composition may have a pH from greater than 5 to 6 and less than 9. The composition may have a pH greater than 5 and less than 8. The composition may have a pH from 6 to 7.5. The composition may have a pH from 9 to 13. The composition may have a pH from 9 to 12 and in another embodiment, the composition may have a pH of from 9 to about 11. The composition may have a pH from 10 to 12.

### Dry Forms of Hypohalous Acid

U.S. Pat. App. 2005/0233900 to Smith et al. describes a dry, powdered form of dilute hypochlorite and hypochlorous acid compositions suitable for use in the invention. Compositions of high water content can be prepared as described in U.S. Pat. App. No. 2003/0160209 to Hoffman et al., U.S. Pat. No. 6,716,885 to Twydell et al., U.S. Pat. No. 5,342,597 to Tunison, III, U.S. Pat. No. 3,393,155 to Schutte et al., and U.S. Pat. No. 4,008,170 to Allan, which are incorporated by reference herein. In accordance with one embodiment of the invention, solutions of dilute hypochlorite are coated using small quantities of treated (hydrophobic) particles by either vigorous agitation or by aerosolization of the solution in the presence of hydrophobic particles to form a solid powder. For example, when hydrophobic fumed silica particles, for example Cab-O-Sil TS-530®, are sheared in the presence of 100 ppm hypochlorite solution in approximately a 95:5-weight ratio of solution to silica, a dry powder can form. Also, a weight ratio of 80:20 can be utilized. The hydrophobic silica forms a porous coating of insoluble fine particles around the solution. Alternately, other colloidal particles or nanoparticles, such as alumina clays, could be treated with a hydrophobic chemical to alter their surface characteristics and then used to encapsulate the hypochlorite solutions. The inorganic thickener can be any natural or synthetic clays, aluminas, etc. One suitable class of thickeners include colloid-forming clays, for example, such as smectite and/or attapulgite types. Smectite clays are more commonly known as bentonite or magnesium aluminium silicate

Fumed silica is formed by burning a volatile silicon compound. This forms primary particles of a few silicon oxide units with a size about 10 nm. These primary particles fuse together to form aggregates with a particle size on the order of 200 nm. These aggregates associate to form agglomerates that are bound by long-range intermolecular forces such as van der Waals forces. The agglomerates have typical particles sizes between 5 and 100 μm. In order to coat water droplets, about 50% or more of the surface silanol groups are typically blocked so they can not ionize, form hydrogen bonds, or otherwise interact with water. The most common approach is to react the silanol groups with silylating agents such as hexamethyldisilazane or polydimethylsiloxane. This converts the surface silanol groups into trimethylsilyl groups. Other agents that are commonly used to block surface silanol groups include trimethylchlorosilane, dimethyldichlorosilane, octamethylcyclotetrasiloxane, alkylsilanes (e.g. octylsilane and hexadecysilane), vinylsilanes (e.g. acrylsilane and methacrylsilane), and similar compounds. The surface silanol groups can also be blocked by association with organic cations or organic polycations (e.g. long chain alkyl amines, quaternary ammonium compounds, or carbamates); by association with polyvalent cations that are also ionically bound to organic ligands (e.g. aluminum stearate, chromium oleate, chromium methacrylate and other metal ions that are complexed to soaps or other anionic organic compounds); by esterification with alcohols or phenols (e.g. methanol, isopropanol, n-butanol, diazomethane, and many other similar compounds); and by association with various types of organic polymers (e.g. polymers formed on a silica surface using polyisocyanate and a polyol, using aldehydes, or using carbodiimides). Non-limiting examples of these quaternary compounds include benzalkonium chlorides and/or substituted benzalkonium chlorides, di(C₆-C₁₄)alkyl di short chain (C₁₋₄ alkyl and/or hydroxyalkl) quaternaryammonium salts, N-(3-chloroallyl) hexaminium chlorides, benzethonium chloride, methylbenzethonium chloride, and cetylpyridinium chloride. Other quaternary compounds include the group consisting of dialkyldimethyl ammonium chlorides, alkyl dimethylbenzylammonium chlorides, dialkylmethylbenzylammonium chlorides, and mixtures thereof. Biguanide antimicrobial actives including, but not limited to polyhexamethylene biguanide hydrochloride, p-chlorophenyl biguanide; 4-chlorobenzhydryl biguanide, halogenated hexidine such as, but not limited to, chlorhexidine (1,1′-hexamethylene-bis-5-(4-chlorophenyl biguanide) and its salts are also in this class. There are three principal suppliers of quaternary based antimicrobials that are registered as actives for this type of use with the EPA. These companies are Lonza, Stepan and Mason Chemical Company. The trade names under which they are marketed are Bardac, BTC and Maquat respectively.

Generally, at least 50% of the surface silanol groups need to be blocked. However, decreasing the amount of unblocked surface silanol groups increases the maximum ionic strength and the maximum pH that can be tolerated. The pKa for treated fumed silica is unknown, but results for silica gel shows that as the surface silanol groups are partially neutralized, the pKa of the unneutralized groups increase. In other words, while the pKa of polymeric silica gel is about 6.5, as the silanol groups are neutralized the pKa of the remaining silanol groups approach the first dissociation constant for mono silicic acid (pH 9.8). With treated fumed silica, the pKa could be higher since the dissociation of the second, third, and fourth hydrogens of silicic acid have pKas of about 12-13. In all of these cases, increasing ionic strength would be expected to decrease the pKa and increase the ionization of unblocked silanol groups. Since data is not available for treated silicas, it has to be confirmed that pH and ionic strength have an impact on particle formation. Also, the critical values of pH and ionic below which particles can be formed with a specific type of treated fumed silica must be empirically determined.

Particles or powders of aqueous solutions coated by hydrophobic materials may be dry blended with various other dry or powdered materials. Separate particles containing incompatible ingredients can be mixed together. Said particles can be formed by coating separate solutions with hydrophobic silica. Other types of particles can be mixed with particles formed by coating solutions with hydrophobic silica. These other types of particles include ingredients that are coated with polymer shells that can be formed by a variety of techniques, including ingredients that are embedded in a matrix such as spray dried starch or sugar, co crystallized with another component such as sugar, absorbed onto a solid support such as fumed silica, zeolite, low density sodium carbonate, puffed borax, etc, or incorporated into polymer beads by absorption or during polymerization, etc. The other ingredients may also be used in solid forms such as powders, crystals, etc.

Ingredients that do not affect the wetting of treated fumed silica by water can be included in the solution that is being coated limited only by solubility and compatibility with other ingredients. Other ingredients such as surfactants and solvents that may affect the interaction of the treated silica and water may be added to the solution in amounts that do not interfere with the ability of the hydrophobic fumed silica to coat the water droplets. The tolerance for these interacting ingredients depends on the type of silylating agent used to treat the silica, the number of unblocked silanol groups, the nature of the ingredient, the ionic strength, and the pH of the solution.

### Silica and Silicate Carriers

The silicas and silicates can be dried by a spray drying technique to obtain particles that are substantially spherical, have a size anywhere from about 50 to about 150 μm. Spray dried precipitated silicas may also be ground so that the densities will vary anywhere from about 80 g/l to about 270 g/l, and the particle size anywhere from about 4 μm to 100 μm. Precipitated silicas and silicates can also be dried by standard drying processes, for example in turbo-driers or rotating driers. Silicas and silicates dried in this conventional way must always be subsequently ground. The tapped density in this regard can be from about 80 g/l to about 240 g/l, and the particle size from about 4 μm to about 15 μm.

Silicas can also be produced by means of a high temperature flame hydrolysis during which silicon tetrachloride is hydrolyzed in an oxyhydrogen flame, which is sometimes referred to as pyrogenic silica. The tapped density of these silicas is somewhere around 50 g/l. Both the precipitated silicas and the pyrogenic silicas can be post-treated in a secondary stage in order to change the naturally hydrophilic surface to a hydrophobic surface, e.g. by a suitable chlorosilane to react with a silanol group on the surface of the silica.

Suitable silicas include hydrophilic silicas having a surface area of from about 50 to 450 m²/g, an average agglomerate size of from about 3.5 to about 100 μm, or an average primary particle size of from about 12 to 30 nm, a tapped density of from about 50 to 240 g/l, a pH of from about 3.6 to about 9, and a DBP adsorption of about 160 to 335 g/100 g. Suitable silicates may comprise those that have a surface area from about 30 to about 40 m²/g, an average agglomerate size of from about 4 to about 6 μm, a tapped density of from about 285 to 315 g/l, a pH of from about 9.5 to about 10.5, and a DBP adsorption of from about 150 to about 170 g/100 g. The other inorganic carriers will also have substantially the same surface area and particle size, although the density will vary depending upon the material employed. Larger surface areas and particle sizes can also be utilized. Extruded films that are water-soluble or water-permeable can also be effective carriers in certain formulations.

Suitable carriers are silicon dioxide, precipitated silica, fumed silica, silicates, bentonite, synthetic hydrated silicon dioxide, diatomaceous earth, clays, attapulgite, hectorite clay, montmorillonite clay, silica gel particles, zeolite (natural or synthetic), kaolinite, smectite, illite, halloysite, vermiculite, sepiolite, beidelite, palygorskite, talc, metal oxides, etc. and mixtures thereof. Synthetic silicon containing particles are suitable, as it enables a good control of the particle size.

Carrier particles can form agglomerates and the average primary particle size is the size of the agglomerated particle. Precipitated silica materials usually appear in the form of agglomerates. The average agglomerate size of the silica range from about 50 to 100 microns. The silica agglomerates may be milled by various known methods to reduce the agglomerate size to the range of 2 to 15 microns. The pH of the silica is normally from about 5.5 to about 7.0.

The hydrophilic silica can also be a fumed silica. Hydrophilic precipitated silica materials useful herein are commercially available from Degussa Corporation under the names SIPERNAT® 22S, 22LS, 50S. Suitably, the silica gel is in the form of particles. The silica gel particles have an average pore diameter, suitably, from about 8 nm to about 10 nm, and a particle diameter of from about 1 mm to about 5 mm.

The smectites produce thixotropic, pseudoplastic dispersions with yield value. These clays are available in a range of viscosities, although their primary functions is to impart yield value and thereby stabilize emulsions, suspension, and foams. They are often used in combination with anionic and nonionic organic thickeners to finely tailor rheology and for advantages synergism in viscosity and/or yield value. The hormites are water dispersible clays with a chain structure that results in microscopic, needle-like particles. The commercial varieties are palygorskite, more commonly known as attapulgite, and sepiolite. The primary commercial palygorskite, attapulgite has typically short (less than 2 um) and low aspect ratio (less than 10:1) needles. When hormite clays are dispersed in water, they do not swell like smectites, but deagglomerate in proportion to the amount of shear applied, and form a random colloidal network. This loosely cohesive structure offers rheological properties similar to those of smectite clays but often with somewhat less physical stability.

### Additional Actives

Additional actives that can be delivered include, for example, a surfactant, a perfume, a fragrance, an insect repellent, a fumigant, a disinfectant, a bactericide, an insecticide, a pesticide, a germicide, an acaricide, a sterilizer, a deodorizer, a fogging agent, and mixtures of these. These actives can be delivered with the hypohalous acid, in a separate vapor stream, or as separate vapors. Suitable fragrances for delivery are described in U.S. Pat. App. 2003/0024997 to Welch et al., which is incorporated herein.

Incompatible actives can be delivered by separating them from the hypohalous acid generator. Fragrances that are sensitive to oxidizing solutions can be added and dispersed into the atmosphere by using individual, replaceable cartridges that liberate the fragrance when heated. Other incompatible actives can be delivered in the same way.

Additional actives that can be delivered with the humidifier include, for example, a perfume, a fragrance, an insect repellent, a fumigant, a disinfectant, a bactericide, an insecticide, a pesticide, a germicide, an acaricide, a sterilizer, a deodorizer, a fogging agent and mixtures of these. These actives can be delivered with the dilute hypohalous acid, in a separate vapor stream, in a mixed vapor stream, or as alternating vapors. Suitable fragrances for delivery are described in U.S. Pat. App. No. 2003/0024997 to Welch et al., which is incorporated herein.

Fragrances, or other incompatible actives that are sensitive to oxidizing solutions can be added and dispersed into the atmosphere by using individual, replaceable cartridges that liberate the fragrance when heated. Other incompatible actives can be delivered in the same way.

### Preparation of Solid Compositions

Compositions can be prepared as described in U.S. Pat. App. 2003/0160209 to Hoffman et al., U.S. Pat. No. 6,716,885 to Twydell et al., U.S. Pat. No. 5,342,597 to Tunison, III, U.S. Pat. No. 3,393,155 to Schutte et al., and U.S. Pat. No. 4,008,170 to Allan, which are incorporated by reference herein. In accordance with the invention, solutions of dilute hypochlorite are coated using small quantities of treated (hydrophobic) particles by either vigorous agitation or by aerosolization of the solution in the presence of hydrophobic particles to form a solid powder. For example, when hydrophobic fumed silica particles, for example “Cab-O-Sil TS-530® are shaken in the presence of 100 ppm hypochlorite solution in approximately a 95:5-weight ratio of solution to silica, a dry powder can form. Also, a weight ratio of 80:20 can be utilized. The hydrophobic silica forms a porous coating of insoluble fine particles around the solution. Alternately, other colloidal particles or nanoparticles, such as alumina or clays, could be treated with a hydrophobic chemical to alter their surface characteristics and then used to encapsulate the hypochlorite solutions.

Free flowing powders containing at least 90% of aqueous solutions of sodium hypochlorite or hypochlorous acid and other optional water soluble salts, buffers, and pH control agents can be formed by mixing said solutions with hydrophobic fumed silica. Suitable hydrophobic fumed silica typically have at least 50% of the silanol groups in the parent fumed silica converted to alkyl siloxy groups or otherwise blocked so they can not interact with water. Further reducing the number of surface silanol groups increases the maximum pH and the ionic strength of the solution that can be coated by the hydrophobic fumed silica. The particles of powdered hypochlorite form spontaneously when the solution is mixed with the silica using enough shear to form water droplets less than about 20 μm in diameter and to break apart the weakly associated silica agglomerates into their fused aggregates of primary particles. The resulting particles break apart when rubbed against a surface to release hypochlorite. Thus, they may be used to clean and to disinfect articles and surfaces. This includes household surfaces and laundry. The hydrophobic silica particles may also have cleaning benefits, either as an abrasive, or by absorbing oils and hydrophobic soils.

The particles do not release hypochlorite until they are disrupted which allows careful control of where they are applied to prevent damage to sensitive areas. They could be applied with a pen-type applicator or some other device. The particles are small enough to adhere to nonwoven material to form hypochlorite-impregnated cleaning wipes or a disposable head for a cleaning wand. The particles can be dispersed in an organic phase such as a cream or a nonaqueous lotion to provide sanitization of hands or removal of odors from feet or underarms. The particles allow the escape of hypochlorous acid vapor, so they may be used as a source of volatile disinfectant which may be used to control odors and the growth of microorganisms, including mold and bacteria, on food in food storage containers, on articles stored in bags, dressers, closets, etc., on dirty laundry stored in hampers, diapers stored in diaper pails, on trash or garbage in waste containers, and on animal litter such as cat litter. In addition to controlling inhibiting the growth of microorganisms, the hypochlorous acid vapors also prevent odors due to the growth of microorganisms as well as modifying odor-causing substances so that they no longer cause undesirable odors. The hypochlorous acid vapor can also deactivate allergens, for example, by deactivating the allergen or allergen generating species. Since hypochlorous acid vapor destroys allergens, the particles may be particularly useful for treating carpets, upholstery and drapery. The particles are small enough to be applied from an aerosol dispenser as well as a shaker can. Combining the ability of allergen destruction and the release of hypochlorous acid may reduce airborne allergens in the vicinity of pet areas such as bird or rodent cages, dog kennels, and cat boxes.

The particles also expand the possibility of formulating hypochlorite-containing products with other ingredients. The dry particles can be combined with a variety of other dry ingredients that will may or may not be kept separate until used. When used the particles will rupture and allow the hypochlorite solution to mix with the other components. These components may only be stable for a brief period when mixed with hypochlorite. They other components could also destroy the hypochlorite after a desired contact time to prevent residual odors or to protect sensitive surfaces from excess exposure to hypochlorite. The destruction could be accomplished by the slow release of a reactive substance such as a reducing agent or a pH control agent that controls the reaction rate with another substance 200 ppm, or from 50 ppm to less than 100 ppm, or between 100 ppm to about 600 ppm available chlorine, or between 100 ppm to about 500 ppm available chlorine, or between 100 ppm to about 400 ppm available chlorine, or between 400 ppm to about 500 ppm available chlorine.

The amount of available halogen oxidant in the composition is determined by placing samples of the composition into about 50 milliliters of distilled water, followed by addition of about 10 milliliters of a 10 weight/weight percent solution of potassium iodide and addition of about 10 milliliters of a 10 volume percent solution of sulfuric acid, the resulting mixture being well stirred. A surfactant that does not react rapidly with hypochlorous acid can be added to facilitate the release of hypochlorite from the particles. The resulting yellow to brown solution, whose color is the result of oxidation of free iodide ion (I⁻) to molecular iodine (I₂), is then volumetrically titrated to an essentially colorless endpoint by addition of standardized 0.01 or 0.1 Molar sodium thiosulfate (Na₂S₂O₃) titrant-Calculation then expresses the result as percent of available molecular chlorine (Cl₂), that is to say assigning two equivalents per mole of titrated hypohalite oxidant. Stability results are then expressed by repeated assays over time using identically prepared samples resulting from the same composition, normalized to 100 percent representative of the starting available chlorine measured initially.

### Hypohalous Acid Vapor

Hypohalous acid vapor can be formed from a variety of oxidants, including compositions containing hypohalite or hypohalous acid, including sodium hypochlorite and hypochlorous acid. Suitable hypohalous acids and salts may be provided by a variety of sources, including compositions that lead to the formation of positive halide ions and/or hypohalite ions, hypohalous acid, hypohalous acid salt, hypohalous acid generating species, hypohalous acid salt generating species; as well as compositions that are organic based sources of halides, such as chloroisocyanurates, haloamines, haloimines, haloimides and haloamides, or mixtures thereof. These compositions may also produce hypohalous acid or hypohalite species in situ. Suitable hypohalous acids and salts for use herein include the alkali metal and alkaline earth metal hypochlorites, hypobromites, hypoiodites, chlorinated trisodium phosphate dodecahydrates, potassium and sodium dichloroisocyanurates, potassium and sodium trichlorocyanurates, N-chloroimides, N-chloroamides, N-chlorosulfamide, N-chloroamines, chlorohydantoins such as dichlorodimethyl hydantoin and chlorobromo dimethylhydantoin, bromo-compounds corresponding to the chloro-compounds above, and compositions which generate the corresponding hypohalous acids, or mixtures thereof.

In one embodiment wherein the compositions herein are liquid, said hypohalite composition comprises an alkali metal and/or alkaline earth metal hypochlorite, or mixtures thereof. Compositions may comprise an alkali metal and/or alkaline earth metal hypochlorite selected from the group consisting of sodium hypochlorite, potassium hypochlorite, magnesium hypochlorite, lithium hypochlorite and calcium hypochlorite, and mixtures thereof. Oxidized water, containing low available chlorine concentrations can be produced by the electrolysis of an aqueous saline solution as described in U.S. Pat. App. 2002/0182262 directed to Selkon.

The anodic oxidation of chloride in an electrolysis cell results in the production of a number of oxychlorine ions including hypochlorite, chlorite, chlorate, and perchlorate. Chlorite is readily oxidized to chlorate. Perchlorate may be an undesirable contaminant in the environment due to its low reactivity, high mobility, and inhibition of thyroid function. The production of hypochlorite via chlorination of caustic water is not believed to result in the formation of perchlorate. This route may be advantageous for certain uses where minor amounts of perchlorate would be undesirable.

### Control of Microbiological Contaminants

In one aspect, the present invention provides a method for controlling a microbiological contaminant. The method generally includes the step of exposing a microbiological contaminant to a gas, e.g. hypochlorous acid, thereby controlling the microbiological contaminant. In one embodiment, the microbiological contaminant can be found in the air. In one embodiment, the microbiological contaminant can be found on a porous surface, such as tile grout, plaster, drywall, ceramic, cement, clay, bricks, stucco, caulking, heating, ventilating, and air conditioning (HVAC) system ducting, ductwork, insulation, and plastic. The microbiological contaminant can be found on a textured surface, such as wallpaper, fabric, tiles, cement, and vinyl flooring. The microbiological contaminant can also be found in other types of interstices or voids, including those defined by heating and/or cooling fins, filters, vanes, baffles, vents, crevices in walls or ceilings, paper and wood products such as lumber, paper, and cardboard, woven products such as blankets, clothing, carpets, drapery, insulation, ceiling tiles, floor coverings, HVAC system, ductwork, shoes, insulation and the like.

The microbiological contaminants can include a mold, mildew, a bacterium, a fungus and/or a virus, e.g. Aspergillus niger, stachybotrys, and penicillin digitatum. The control encompassed by the present invention can include cleaning, sanitizing, deodorizing, sterilizing, or killing target microbiological contaminants. This control can include killing a mold spore population and/or a mold population. The method can include controlling one or more microbiological contaminants in a bedroom, bathroom, kitchen, refrigerator, toy box, play area, storage area, restaurant, gym, medical facility, locker room, or aquatic facility. The present invention can be used for a variety of applications, including delivery of a gas to residential and commercial surfaces, and for a variety of purposes including, but not limited to disinfecting, deodorizing, bleaching, sanitizing, and sterilizing.

### Forms

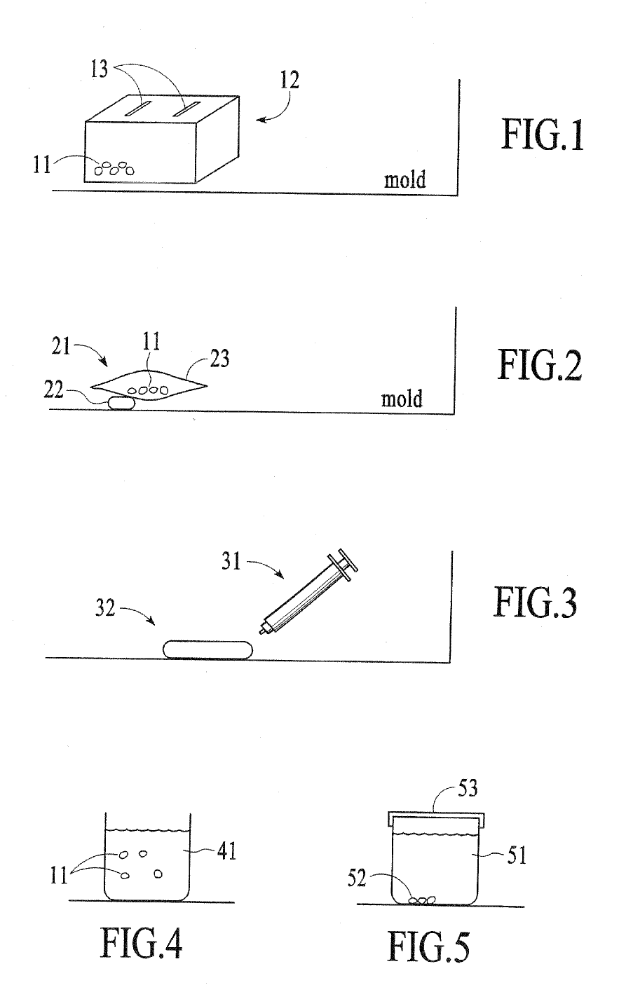

Aqueous solutions made from sodium hypochlorite emit sufficient amounts of hypochlorous acid vapor and possibly other available chlorine compounds (e.g. dichlorine monoxide and chlorine) to disinfect or prevent the growth of microorganisms on surfaces in contact with the vapor. As shown in FIG. 1, a liquid composition can be converted to a solid 11 to make it easier to contain the composition within a container 12 that emits the hypochlorous acid vapors through openings 13 to control mold or other microbiological contamination at a remote location. For example, the solution can be absorbed onto a mass of fibers or a porous solid such as puffed borax, fumed silica, or clay. Such solids may be free flowing or not depending on the ratio of liquid to absorbent. Free flowing solids can be made by mixing the aqueous solution with hydrophobic fumed silica. Hypochlorite solutions may also be encapsulated or microencapsulated using various shell-forming materials. In addition to the above containers, solids 11 or other forms can also be incorporated into pouches or sachets 21 made of woven or nonwoven materials, as shown in FIG. 2. Clays such as Laponite® can also be used to convert the liquid solution into a gel. Gels may be incorporated into any of the above containers or delivery systems. In addition, gels 32 may be applied to a surface using an applicator such as a syringe 31, as shown in FIG. 3. Solids may be sprinkled on a surface. These powders and gels will then emit the hypochlorous acid vapors into the space where microbial control is desired. Solutions may also be absorbed onto pads or nonwovens from which the vapors are emitted similar to some air fresheners. Solid carriers 11 may also be incorporated into wax gels 41, as shown in FIG. 4, from which the hypochlorous acid vapors are slowly emitted.

In one embodiment, the gel includes volatile waxes such as cyclotetradecane. Solutions 51 may also be in equilibrium with solid hypochlorite releasing materials 52, as shown in FIG. 5, to prolong the life of the emitter. For example, dichlorohydantoins have a solubility limit that results in a sodium hypochlorite concentration of several hundred ppm. Excess dichlorohydantoin will remain as a solid that dissolves to replenish the hypochlorite as it is emitted as hypochlorous acid vapor.

### Carriers

The carriers may take any shape or form, including particles, agglomerates, granules, pellets, briquets, continuous sheets, discontinuous sheets, films, coatings, extruded rods, tubes, and the like. Granules, pellets, or briquets comprise suitable carrier shapes and sizes although water vapor can permeate the layer, and refers to materials that allow permeation of liquid water as water permeable. Suitable water vapor selective materials can be made from a variety of materials including, but not limited to, polytetrafluoroethylene (PTFE), polypropylene (PP), polyethylene (PE), and fluorinated ethylene propylene (FEP). Some water vapor selective materials are applied to a web that provides structural integrity to the material, e.g. where the material is thin and requires support to prevent tearing during manufacture and use.

Examples and embodiments of the materials and apparatuses described herein are also disclosed in U.S. Pat. Nos. 6,607,696 and 6,602,466, as well as PCT Publication No. WO 03/05146, all entitled “Methods and Apparatus for Controlled Release of a Gas,” the entire disclosures of which are incorporated in their entirety by this reference.

### Containers and Pouches

The vapor emitting composition may be a liquid, gel or solid in a container with one or more openings or perforations to allow the vapor to escape. Optionally, the opening may have a membrane or film 53 that is at least partially permeable to the hypochlorous acid vapor, as shown in FIG. 5. The composition, a liquid, gel or solid, may also be contained in a pouch made from a membrane or film that contains the composition but allows the vapors to pass. Generally, discrete amounts of actives disposed within a device such as a pouch, can control microbiological contaminants in a target area. The device can be affixed with an adhesive strip 22 (FIG. 2) or other fastening device to the surface to expose the microbial contaminants to the gas. In one embodiment, the active is substantially sealed in a pouch (e.g. a sachet) that includes a gas permeable layer. The gas permeable layer can be any permeable layer, e.g. a water vapor selective material or any of the permeable layers described herein. The sachet or pouch can wholly be constructed from gas permeable layers, or the gas permeable layer can comprise only a portion, e.g. one side 23 (FIG. 2) of a sachet. The remainder of the sachet or pouch can include impermeable materials or other materials, such as sachet layers forming an impermeable area. The device can also include additional elements such as additional sachets or one or more envelopes.

Suitable permeable and selective transmission films include 8181-G from Bemis® (OPET/adhesive/LLDPE), a film from American Packaging Corp. (PET/ink/adhesive/LLDPE), 24CTN from Exopack® (PET), a film from Alcan® (LLDPE), OW-134.5 from Pliant® Corp. (MDPE), GF-14 from Pliant® Corp. (LDPE), X5-202-315.2 from Pliant® Corp. (LLDPE/EVOH/m-PE), and GX-P from Pliant® Corp. (AlOx coated PE). Other suitable films include fluoropolymer films from W.L. Gore®.

The device can be in the form of a surface patch that generates a gas (e.g. hypochlorous acid), which diffuses across a permeable membrane (e.g. a water vapor selective layer), and migrates into the porous surface controlling the microbiological contaminant (e.g. mold and/or mold spores). In one embodiment, the patch includes an impermeable layer on the side of the apparatus to be placed opposite the surface to be treated. The utilization of an impermeable backing prevents the escape of the gas in the opposite direction, instead focusing diffusion to the surface containing the microbial contaminant.

The patch can also include an adhesive layer that faces the contaminated surface. The adhesive or other attachment means can be applied about the entire perimeter or only a portion of the perimeter. Other methods and devices for adhering an apparatus to a surface can also be employed, such as one or more clips, velcro, etc. In a suitable embodiment, the present invention features an apparatus for the generation of hypochlorous acid that is applied to dry wall. However, the present invention can be applied to any number of porous surfaces which may be found, but not limited to, the home, gym, dental and medical equipment, building restoration, food processing plants, and any other areas which would have a surface (e.g., a porous or textured surface), containing a contaminant. Further embodiments include apparatus in the form of a strip for application to selected surfaces and devices that include dispersion devices for application in larger areas, e.g. a room or a portion of a room.

The composition may be contained in a sachet or other porous form of containment that allows vapors such as hypochlorous acid to be released into the environment. The composition may also be adhered to a strip or some other device such as a double-sided adhesive tape for attachment inside containers such as trash cans, closets, drawers, diaper pails, etc. This allows the release of hypochlorus acid or other vapors that control odors, allergens and microorganisms in air or on surfaces. In a suitable embodiment, the apparatus includes an adhesive strip disposed about its perimeter, or a portion of its perimeter.

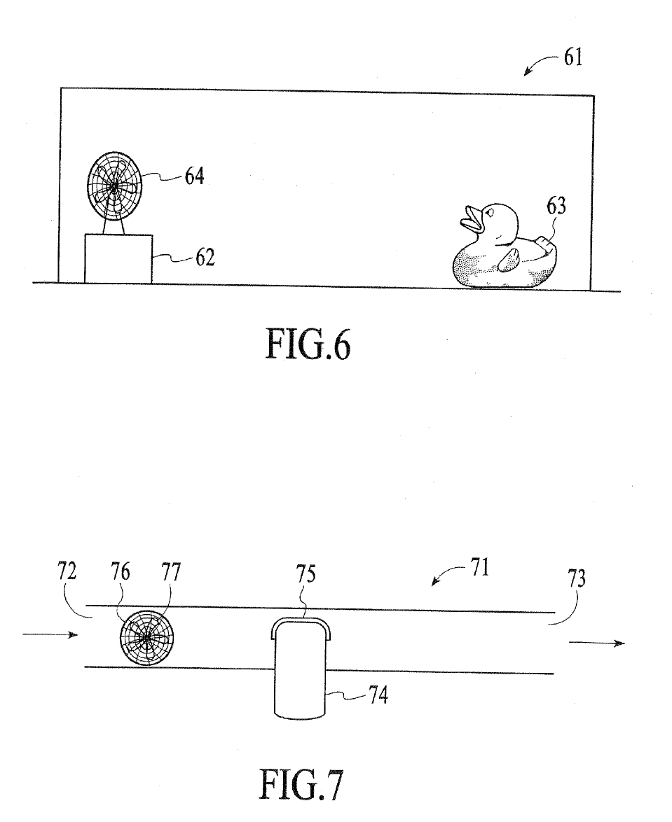

The source of hypochlorous acid vapor may be incorporated as part of an article or container that has a cavity and a door or lid into which items can be placed. Alternatively the source 62 of hypochlorous acid and the container 61 are separate entities which are combined at the time of use, as shown in FIG. 6. In either case, items, such as a toy duck, 63 are placed in the container 61 and the hypochlorous acid vapors either reduce the number of viable organisms on the item or they prevent the growth of microorganisms. This is accomplished without direct contact by the composition that emits the hypochlorous acid vapors. Thus, items such as electronic devices that are sensitive to immersion in water can be decontaminated. Multiple items can also be decontaminated at the same time. The decontamination of the items is done automatically by the vapors without wiping or scrubbing. The continuous nature of the hypochlorous vapor emission prevents the growth of microorganisms over time. This will prevent the growth of mildew and the development of odors in clothing and other items stored for a prolonged period. It will also preserve food and prolong food freshness in food storage containers. Articles which emit hypochlorous acid vapors may also be placed in various confined spaces such as drawers, closets, hampers, diaper pails, trash cans, toy boxes, and refrigerators. This will provide benefits to the contents of these confined spaces as discussed above.

Articles which emit hypochlorous acid vapors may also be used in rooms to disinfect surfaces or hinder the growth of microorganisms. They may be useful in showers to hinder the growth of mold and mildew or the growth of athelete's foot fungus. They may be placed near toilets to continuously disinfect surfaces such as the handle. This would also apply to other high touch areas such as door knobs. They may be used in doctor's offices to slowly disinfect environmental surfaces overnight with less effort than traditional disinfectants. The vapors may also be more effective at decontaminating difficult to reach places and irregular surfaces. The hypochlorous acid vapors can react with and neutralize many odorous compounds for odor control. These include compounds with sulfide, sulfhydryl, alkene, alkyne, aldehyde, ketone, amine, amide, nitrile and similar reactive groups, such as described in U.S. Pat. No. 6,749,805 for the deodorization of flatus.

The vapor emitting articles may be any form suitable to be hung using tape or hooks or they may be constructed to be set on a floor or other surface. They may have any shape and size. They may have mechanical louvers or vents to control the emission of the hypochlorous acid vapors or they may be placed inside containers with covers that screw or slide to form an opening of variable size.

Since the loss of hypochlorous acids during product distribution will affect the useful life of the product, it will be advantageous to have a product that is sealed during distribution and activated before use. This could be as simple as a tight fitting closure on a bottle or an overwrap of a barrier film on a pouch or sachet. The hypochlorous acid could be generated in situ by electrolysis. It can also be formed in-situ by altering pH. Alkaline hypochlorite solutions above about pH 11 are primarily composed of sodium hypochlorite which is not volatile. At the time of use the product could be activated by adding an acid to reduce the pH to where an effective amount of hypochlorous acid can be released. This could be done by adding a liquid or a powder to the solution or by removing or breaking a barrier that separates the two substances and allowing them to mix. An example is two compartments of a pouch or sachet that are separated by a film or valve that is broken or opened by applying pressure, vacuum, or some other physical means. Another approach would be to add water to a solid such as dichlorohydantoin, which results in at least partial hydrolysis of the solid to form a solution that contains hypochlorous acid forms of hypochlorite and is hereby incorporated by reference in its entirety. Suitable carriers can comprise silicas and silicates. Precipitated silicas employed in this regard are produced from solutions of water glass into which sulfuric acid is introduced under fixed conditions. They are formed in the aqueous phase, and depending on the conditions of precipitation, it is possible to produce products with smaller or somewhat larger primary particles, which then basically determine particle size and specific surface area. The precipitates obtained are then washed and dried by methods known in the art. Silicates are also manufactured by a precipitation method, however, the acids which are necessary for precipitation may be replaced partially or completely by solutions of metallic salts such as aluminum sulfate, and the like. The precipitation parameters can also be adjusted to suit the various raw materials.

### Suitable Hypochlorous Acid Devices Substantially Free From Chlorine

Chlorine and chlorine dioxide vapors inhibit mold and kill bacteria, however, they also discolor dyes on fabrics and have relatively higher toxicity than hypochlorous acid, which makes chlorine dioxide and chlorine less desirable. Solutions that emit hypochlorous acid vapors can be modified to reduce or eliminate the co-emission of chlorine. Several approaches can be effective in mitigating the release of chlorine and chlorine dioxide and the discoloration of fabrics.

One approach is the reduction or elimination of water vapor, for example, using a desiccant. Chlorine does not absorb readily onto dry fabrics. The desiccant can be in a larger container that surrounds the container from which the hypochlorous acid vapors are emitted. The desiccant can also be sandwiched or otherwise contained within permeable or perforated plastic films that are used to cover the emitting container. Semi-permeable films, membranes or nonwovens that allow hypochlorous acid vapors to largely permeate, but restrict the release of water vapors may also be used (e.g. Gore-Tex®. films). The role of water can be demonstrated by comparing damage on dry fabric, fabric equilibrated at 80° F./80% relative humidity, and fabric soaking wet. The soaking wet fabric shows the worst dye damage.

Another approach is increasing the pH of the bleach solution to reduce chlorine. See in the Examples Section below. An isobaric line for constant hypochlorous acid vapor pressure can be calculated from literature values of various equilibrium constants for various concentrations of sodium hypochlorite and pH. Thus, one can maintain the performance of a desired concentration of hypochlorous acid but eliminate chlorine by increasing pH and hypochlorite concentration according to the isobaric line. This is just an example since other partial pressures of hypochlorous acid are also effective and may be more effective depending on the size of the container, etc. The higher bleach concentrations also allow for smaller volumes of solution since the volume of solution required to provide a certain number of moles of hypochlorous acid decreases as the concentration increases. These more concentrated solutions also maintain a more stable concentration with time because the amount of hypochlorous acid vapor emitted per hour is a much smaller fraction of the total amount of bleach than in a more dilute solution. This allows much longer product lifetimes for an emitter. For example, using 6000 ppm sodium hypochlorite at pH 9 provides continuous disinfection for more than a month in which contaminated slides are exposed and then evaluated every couple of days to confirm ongoing efficacy. In one embodiment, the sodium hypochlorite at about pH 9 is gelled using clay.

Another approach is minimizing the amount of vapor emitted. In this case the dose of vapor must be sufficient to kill microbes but not damage fabric dyes. In one embodiment, this can be done using a small volume of sodium hypochlorite solution at a dilute concentration at low pH. For example, 50 g of a 200 ppm sodium hypochlorite solution at pH 5.5 did not discolor fabrics in a 14 L container. However, essentially all the bleach was emitted from the solution in a relatively short period of time.

Another approach is using a filter to remove chlorine from the vapor leaving the emitter. Covering the emitter with nylon or with polyester fabric prevented the discoloration of fabric dyes. Unfortunately, the nylon also absorbed most of the hypochlorous acid as well and the vapor was not as effective at killing microorganisms. With polyester, the vapors were still an effective biocide and the vapor concentration (as measured electrochemically) was only partially reduced. Other polymers may also selectively remove chlorine from the vapor.

Another approach is using a fan 64 or spray to better disperse the vapors throughout the container 61, as shown in FIG. 6. There appears to be a non-linear concentration gradient of vapor as fabrics close to the emitter experience greater dye discoloration than those further away, but after some distance the fabric damage is essentially constant. This gradient was also confirmed by measuring the bleach absorbed into water at varying distances from the emitter. In addition, combinations of various approaches may also be effective.

Additional volatile agents may also be effective biocides. Examples include diacetyl, maltol, t-butyl hypochlorite, and hydrogen peroxide. With hydrogen peroxide vapors acceptable disinfection is achieved in closed containers with aqueous solutions that contain more than about 0.5% hydrogen peroxide, although the lower concentrations have some activity. Solid, nonvolatile compounds that contain an active halogen such as N-halohydantoins can also emit effective vapors by various means including equilibria with volatile chlorine containing species, hydrolysis with water vapor present in air, and auto decomposition. Such compounds can also be combined with solid acids or bases or other reactants to promote or regulate the formation of effective vapors.

### Replaceable Cartridges

The device can have replaceable or disposable cartridges containing concentrated or dilute hypohalous acid in liquid or solid form that are readily placed in the device. The replaceable cartridges can also be generators of hypohalous acid, such as by electrolysis or hydrolysis. The replaceable cartridges can also deliver additional ingredients.

### Portable Devices and Powered Devices

The device can contain an energy source, such as batteries, and can also contain a means for allowing recharging of rechargeable internal batteries via such means as a plug or port such that the consumer can conveniently recharge the batteries. Other means of providing energy sources that allow the device to be portable include methanol fuel cells or minerals that generate heat upon mixture with water, for example, mixing water with anhydrous calcium oxide. Portable devices would allow for disposable dispersion devices that could be taken for “on the go” occasions. For example, such systems could fit in the cup holders of vehicles.

In one embodiment of the device, the battery, fan, motor, and circuitry are designed to require a very low power draw, enabling the device to run continuously for a long period of time. Suitably, this embodiment of the device continuously draws less than 20 mA, or less than 10 mA, or less than 8 mA. To avoid the need for frequent battery replacement, the replaceable power supply of this embodiment preferably is designed to last at least one month, or at least two months, or at least three months, or at least four months.

### Product Containers for Delivery of Dilute Hypochlorite Solutions

Any container adapted to deliver a spray of droplets as defined herein is suitable for use herein. Several modifications can be made to the conventional, single aperture, spray head to ensure that a spray of such droplets as required herein is formed. Suitable containers to be used herein (also called “spray dispensers”) share the common feature of having at least one aperture or a plurality of apertures also called “dispensing openings” through which the composition is dispensed so as to produce the spray of droplets as defined herein. Examples of suitable containers are disclosed in U.S. Pat. App. 2005/0221113 to Bitowft et al., which is hereby incorporated within.

The container herein can comprise a spray dispenser. The composition may be dispersed into the air. The composition may be dispersed using an atomizer, an ultrasonic sprayer, a humidifier, a vaporizer, a nebulizer, or a spray device. The composition may be delivered on a continuous basis, such as with a humidifier. The composition may be delivered on a pulsed basis, such as with a canister on a timer. One spray device is an electrostatic sprayer, as described in PCT App. WO01/20988. The composition may be applied to skin surfaces. The composition may be delivered from a variety of containers, such as a dual chambered bottle, a trigger spray bottle, an aerosol canister, and a bleach pen. The composition may be applied as a foam to soft or hard surfaces.

The composition is placed into a spray dispenser in order to be distributed onto the target. The spray dispenser for producing a spray of liquid droplets can be any of the manually activated means as is known in the art, e.g. trigger-type, pump-type, non-aerosol self-pressurized, and aerosol-type spray means, for adding the composition to small surface areas and/or a small number of targets, as well as non-manually operated, powered sprayers for conveniently adding the composition to large surface areas and/or a large number of targets. Suitable manually activated sprayers and non-manually activated sprayers for use with the compositions of the current invention are described, e.g., in U.S. Pat. No. 5,783,544 and U.S. Pat. No. 5,997,759 to Trinh et al., both of said patents are incorporated herein by reference. Additional sprayers are disclosed in U.S. Pat. No. 5,294,025 to Foster; U.S. Pat. No. 4,082,223 to Nozawa; U.S. Pat. No. 4,161,288 to McKinney; U.S. Pat. No. 4,558,821 to Tada et al.; U.S. Pat. No. 4,434,917 to Saito et al.; and U.S. Pat. No. 4,819,835 to Tasaki, all of said patents being incorporated herein by reference.

These spray dispensers may be manually or electrically operated. Typical manually operated spray dispensers include pump operated ones to trigger operated ones. Indeed, in such a container with a spray dispenser head the composition contained in the container is directed through the spray dispenser head via energy communicated to a pumping mechanism by the user as said user activates said pumping mechanism or to an electrically driven pump. In one embodiment, the means for delivering the composition comprises an electrically driven pump and a spray arm being either extended or extendible and having at least one dispensing opening so that in operation, the composition is pumped by electrically driven pump from the container, through the spray arm to the dispensing opening from which it is dispensed. In this embodiment, the spray arm communicates with the container by means of a flexible connector. The spray arm may have one nozzle or multiple nozzles located along its length. The spray arm makes it easier to control where the composition is sprayed. The electrically driven pump may be, for example, a gear pump, an impeller pump, a piston pump, a screw pump, a peristaltic pump, a diaphragm pump, or any other miniature pump. The spray arm may be extensible either by means of telescopic or foldable configuration.

The compositions herein can be used by placing them in an aerosol dispenser. An aerosol dispenser comprises a container which can be constructed of any of the conventional materials employed in fabricating aerosol containers, including plastics, aluminum, and tin plate. The dispenser must be capable of withstanding internal pressure in the range of from about 20 to about 110 p.s.i.g., more preferably from about 20 to about 70 p.s.i.g. The one important requirement concerning the dispenser is that it be provided with a valve member, which will permit the composition contained in the dispenser to be dispensed in the form of a spray of particles or droplets. The aerosol dispenser utilizes a pressurized sealed container from which the composition is dispensed through a special actuator/valve assembly under pressure. The aerosol dispenser is pressurized by incorporating therein a gaseous component generally known as a propellant. Suitable propellants are compressed air, nitrogen, inert gases, carbon dioxide, gaseous hydrocarbons such as isobutene, etc. A more complete description of commercially available aerosol-spray dispensers appears in U.S. Pat. No. 3,436,772 to Stebbins; and U.S. Pat. No. 3,600,325 to Kaufman et al.; both of said references are incorporated herein by reference.

The composition may be stored or shipped in a variety of containers, including glass, ABS, polycarbonate, high density polyethylene, low density polyethylene, high density polypropylene, low density polypropylene, polyethylene terephthalate, or polyvinylchloride. A variety of additives in the container may affect the stability of the composition. For instance, the density of the polyethylene resin may be modified by co-polymerizing with a small amount of a short chain alkylene, e.g., butene, hexene or octene. Various other additives can be added, such as colorants, UV blockers, opacifying agents, and antioxidants, such as hindered phenols, e.g., BHT, Irganox 1010 (Ciba-Geigy A.G.), Irganox 1076 (Ciba-Geigy A.G.), Tonol (Shell Chemical Co.). Mold release agents and plasticizers can be added, especially to other types of plastics. The containers may have barrier films to increase storage stability. Suitable barrier films may include nylons, polyethylene terephthalate, fluorinated polyethylenes, and Barex (a copolymer of acrylonitrile and methylmethacrylate that is available from British Petroleum).

The composition may be prepared by mixing a solid composition with water. The solid composition may be a tablet, granular composition, paste, or other solid composition. The composition may be prepared by diluting a liquid composition with water. The water may be purified. The composition may be prepared by mixing two liquids, for example, from a dual chambered container or a dual chambered spray bottle. The composition may be produced by chemical or electrical means, for example by electrolysis.

The compositions of the invention can be diluted prior to use with tap water or water of higher purity. Preparation of dilute compositions for storage, for example as pre-diluted in bottles, may require water of higher purity. This higher purity water can be obtained by a variety of processes, including for example, distillation, filtering, sodium cation exchange (soft water), hydrogen cation exchange (deionized water without anion exchange), reverse osmosis, activated carbon treatment, ultrafiltration, nanofiltration, electrodialysis, and UV light treatment.

The compositions of the invention can be diluted prior to use from a concentrated liquid or solid composition. For instance, liquid, especially aqueous, sodium hypochlorite optionally containing surfactants or other additives of 5.25% available chlorine concentration can be diluted to below 500 ppm available chlorine concentration. Tablets or powders having solid hypochlorite or hypochlorite generators can be dissolved in water to deliver compositions below 500 ppm concentration. Examples of compositions that can be diluted are described in U.S. Pat. No. 6,297,209, U.S. Pat. No. 6,100,228, U.S. Pat. No. 5,851,421, U.S. Pat. No. 5,688,756, U.S. Pat. No. 5,376,297, U.S. Pat. No. 5,034,150, U.S. Pat. No. 6,534,465, U.S. Pat. No. 6,503,877, U.S. Pat. No. 6,416,687, U.S. Pat. No. 6,180,583, and U.S. Pat. No. 6,051,676. The compositions will typically be diluted with an aqueous liquid, usually tap water, prior to use. When diluted, the compositions comprise from about 40 ppm to about 12,500, preferably from about 50 ppm to about 200 ppm of registered disinfectant.

The compositions of the invention can be delivered as part of a multi-compartment delivery system, for example as described in U.S. Pat. No. 5,954,213, U.S. Pat. No. 5,316,159, PCT App. WO2004/014760, U.S. Pat. No. 6,610,254, and U.S. Pat. No. 6,550,694.

### Plastic Aerosol Container

Several container technologies can improve the stability of dilute hypochlorite compositions. One technology involves changing the materials in contact with the dilute hypochlorite composition. We have surprisingly found that dilute hypochlorite may be stored or shipped in a variety of plastic aerosol containers that offer better stability than metal aerosol containers laminated with plastic film. Another technology involves separating the dilute hypochlorite composition from the propellant or other active ingredients in separate chambers. Another technology option is to create a fine mist without the use of propellant. All three options can improve the stability of dilute hypochlorite compositions. The container can also be electrically powered, for example, as described in U.S. Pat. No. 5,716,007 to Nottingham et al., U.S. Pat. App. 2002/0055176 to Ray. Plastic aerosol containers can improve stability of dilute hypochlorite. The plastic container may be composed of any thermoplastic material that may be formed into the desired shape. Examples of such materials include ethylene based polymers, including ethylene/vinyl acetate, ethylene acrylate, ethylene methacrylate, ethylene methyl acrylate, ethylene methyl methacrylate, ethylene vinyl acetate carbon monoxide, and ethylene N-butyl acrylate carbon monoxide, polybutene-1, high and density polyethylene, low density polyethylene, polyethylene blends and chemically modified linear low density polyethylene, copolymers of ethylene and C1-C6 mono- or di-unsaturated monomers, polyamides, polybutadiene rubber, polyesters such as polyethylene terephthalate, polyethylene naphthalate, polybutylene terephthalate; thermoplastic polycarbonates, atactic polyalphaolefins, including atactic, high density polypropylene, polyvinylmethylether and others; thermoplastic polyacrylamides, polyacrylonitrile, copolymers of acrylonitrile and other monomers such as butadiene styrene; polymethyl pentene, polyphenylene sulfide, aromatic polyurethanes; styrene-acrylonitrile, acrylonitrile-butadiene-styrene, styrene-butadiene rubbers, acrylontrile-butadiene-styrene elastomers, polyphenylene sulfide, A-B, A-B-A, A (B-A)n-B, (A-B)n-Y block polymers wherein the A block comprises a polyvinyl aromatic block such as polystyrene, the B block comprises a rubbery midblock which can be polyisoprene, and optionally hydrogenated, such as polybutadiene, Y comprises a multivalent compound, and n is an integer of at least 3, and mixtures of said substances. A suitable thermoplastic material is polyethylene naphthalate, polyethylene terephthalate (PET) and copolymers derived from PET. The thermoplastic polymers used to make the plastic container can be transparent, opaque or partially opaque polymers low density polypropylene, polyethylene terephthalate, or polyvinylchloride. A variety of additives may affect the stability of the composition. For instance, the density of the polyethylene resin may be modified by co-polymerizing with a small amount of a short chain alkylene, e.g., butene, hexene or octene.

The manufacture of thermoplastic parts by melt fabrication processes such as extrusion and molding is generally not possible using neat polymers directly as synthesized. Instead, it is common practice to “formulate” compositions containing a variety of ingredients in relatively small, but critical amounts. These ingredients may be categorized into two main and fairly distinct groups, namely product additives and processing aids. The product additives, which primarily serve the function of modifying the properties of the fabricated material, include pigments, such as titanium dioxide, and dyes (colorants), heat stabilizers and antioxidants, light and UV stabilizers, antistatic agents, slip and antiblocking agents, and the like. The processing aids primarily, if not exclusively, facilitate processing-1-often to the point that processing would be impossible without them. Foremost among these aids are lubricants, sometimes referred to as release agents, which prevent sticking of the hot molten thermoplastic polymer to fabrication surfaces such as extruder screws, extrusion dies, mill and calender rolls, injection molds, and the like. Lubricants are described in U.S. Pat. No. 4,925,890 to Leung et al. Antioxidants, UV absorbers and light stabilizers are described in U.S. Pat. No. 4,972,009 to Suhadolnik et al.

Various other additives include colorants, UV blockers, opacifying agents, and antioxidants, such as hindered phenols, e.g., BHT, Irganox 1010 (Ciba-Geigy A.G.), Irganox 1076 (Ciba-Geigy A.G.), Tonol (Shell Chemical Co.). Mold release agents and plasticizers can be added. The containers may have barrier films to increase storage stability. Suitable plastic barrier films may include nylons, polyethylene terephthalate, fluorinated polyethylenes, and Barex (a copolymer of acrylonitrile and methylmethacrylate that is available from British Petroleum).

Labels may contain an opacifier, colorant, or UV inhibitor, for example, PCT App. WO0132411 to Cole et al. where the label adhesive contains a UV inhibitor.

Multilayer containers are preferred for compositions of the invention. Multilayer containers are described in PCT App. No. WO2004/069667, WO9601213 to Slat et al., PCT App. No. WO0238674 to Pope Share et al., U.S. Pat. No. 5,553,753, PCT App. WO0192007 to Abplanalp including a separate compartment for propellant, U.S. Pat. No. 5,579,944 to Hafner Barger et al. describing plastic gaskets, U.S. Pat. No. 6,474,513 to Burt describing a plastic valve stem, PCT App. 2003/0178432 to Meiland et al. describing an aerosol container with plastic side walls, U.S. Pat. App. 2003/0150327 to Bolden. The treatment may be provided by electrostatic filters, for example, as 6,019,252 to Benecke et al. describing a plastic aerosol container inside a metal sleeve, and U.S. Pat. No. 6,589,509 to Keller et al. describing a plastic aerosol container with a composition containing two phases WO0134479 to Serrano and references cited therein.

Suitable propellants must not cause instability to the dilute hypochlorite. Nitrogen and hydrofluorocarbons, such as 134A and 152A, can give greater stability compared to carbon dioxide and hydrocarbon propellants.

### Containers

The composition may be delivered using a variety of delivery devices, including those described in Co-pending application Ser. No. 11/096,135, Packaging for Dilute Hypochlorite, filed Mar. 31, 2005 to Bitowft et al. The composition may be dispersed using an atomizer, a vaporizer, a nebulizer, a hose with laser created slits, or a spray device. The composition may be delivered on a continuous basis, such as with a humidifier. The composition may be delivered on a pulsed basis, such as with a canister on a timer. One spray device is an electrostatic sprayer, as described in PCT App. WO972883 to Fox et al. The composition may be applied to skin surfaces. The composition may be delivered from a variety of containers, such as a dual chambered bottle, a trigger spray bottle, an aerosol canister, and a bleach pen.

### Dual Delivery Container

Alternatively, such liquid formulations may be provided as a dual container delivery system can comprise a first container containing a first aqueous solution comprising the hypohalite or a source thereof, a second container containing a second aqueous or non-aqueous solution comprising the incompatible active, for example a promoter, surfactant, additional agents, and delivery means for delivering the first and second solutions to a surface such that the hypohalite and incompatible active agents are admixed just before or upon impacting the surface. A suitable kind of embodiment could be realised merely by providing the two solutions in respective separate containers. The consumer could then apply each to the surface, either sequentially or simultaneously. However, it is more convenient to provide the products in a dual-compartment container in which the aqueous solutions are stored in separate compartments. The delivery means, then allows them to be delivered to the surface as they are exiting the delivery means and/or in mid-air as they are directed to the surface and/or on the surface itself. Preferably, they are delivered to be mixed in approximately equal volumes, i.e. typically from 0.5:1 v/v to 1:0.5 v/v. A particularly preferred delivery means, either from a single compartment or a dual compartment container, is a trigger spray head. In the case of a dual compartment system, this will preferably have two siphon tubes, respectively leading into each compartment and either a single nozzle with a mixing chamber or two separate nozzles substantially adjacent to each other. If desired, a dispensing nozzle or nozzles configured to promote foaming may be used. U.S. Pat. No. 6,817,493 to Parsons et al. describes a dual nozzle suitable for an aerosol or other liquid dispensing device.

### Bag-in-Can Technology

In this container design, the product exists in a separate pouch, either foiled or foil-less bag, that is surrounded by propellant, for example, U.S. Pat. No. 6,196,275 to Yazawa et al., U.S. Pat. No. 4,308,973 to Irland, and U.S. Pat. No. 5,730,326 to Kaeser describing a rechargeable container. U.S. Pat. App. 2003/0102328 to Abplanalp et al. describes an aerosol container lacking a return spring and product dip tube. For some applications, a dip tube may still be appropriate. The valve may have multiple product delivery openings. The container may use a propellant driven piston to dispense the product or the product may be in a collapsible, flexible bag.

### Dual Chambered Device

With a dual chambered device, the solution of dilute bleach is separated from the propellant or other additives. This allows additional components that may be incompatible with dilute hypochlorite (fragrance, surfactant) to be in the final delivered composition. U.S. Pat. No. 6,481,435 to Hochrainer et al. and U.S. Pat. No. 4,988,017 to Schrader et al. describe a variety of dual chambered devices.

### Expandable Chamber Device

AQUA U.S. Pat. No. 5,111,971 to Winer describes a pressurized liner-sleeve assembly that can be fitted with an aerosol valve. This technology has no propellant, however, the product must still be stable to the elastomeric sleeve used to form the chamber.

Precompression Trigger

This technology is similar to standard trigger technology, but with a compression chamber that allows the product to be delivered with more force and smaller particle size. U.S. Pat. No. 6,364,172 to Maas et al. and U.S. Pat. No. 5,730,335 to Maas et al. describe a precompression valve in a pumping cylinder of a trigger sprayer which only allows pressurized liquid to be expelled when the pressure of the liquid in the pumping cylinder is above a certain predetermined level.

### Mechanically Pressurized Device

U.S. Pat. No. 6,708,852 to Blake describes a mechanically pressurized dispensing system that offers an alternative to chemically pressurized aerosol dispensers. The system is fitted over a standard container holding a liquid product, and includes a dip tube assembly to draw liquid into the dispensing head assembly, where the contents are released through the dispensing head assembly, via the nozzle and valve. A twist of the threaded cap raises a piston, thereby opening a charging chamber within the dispensing head assembly. This creates a vacuum with the resulting suction pulling the product up through the dip tube to fill the charging chamber. Twisting the cap in the opposite direction lowers the piston in a downstroke, which closes the charging chamber, forcing the product into the expandable elastic reservoir where it is then discharged through the nozzle.

Elimination of the chemical propellant can improve the stability of dilute hypochlorite. Alternatives to chemically pressurized dispensers include various mechanically pressurized models that obtain prolonged spray time by storing a charge without the use of chemical propellants. Such “stored charge” dispensers include types that are mechanically pressurized at the point of assembly, as well as types that may be mechanically pressurized by an operator at the time of use. Stored charge dispensers that are pressurized at the point of assembly often include a bladder that is pumped up with product. Examples include those described in U.S. Pat. No. 6,656,253 to Willey et al. The treatment may provide a variety of treatment mechanisms, for example, as U.S. Pat. Nos. 4,387,833 and 4,423,829.

Stored charge dispensers that are pressurized by an operator at the time of use typically include charging chambers that are charged by way of screw threads, cams, levers, ratchets, gears, and other constructions providing a mechanical advantage for pressurizing a product contained within a chamber. This type of dispenser will be referred to as a “charging chamber dispenser.” Many ingenious charging dispensers have been produced. Examples include those described in U.S. Pat. App. 2004/0047776 to Thomsen. The treatment may provide a chemical means to decontaminate, for example, U.S. Pat. No. 4,872,595 of Hammett et al., U.S. Pat. No. 4,222,500 of Capra et al., U.S. Pat. No. 4,174,052 of Capra et al., U.S. Pat. No. 4,167,941 of Capra et al., and U.S. Pat. No. 5,183,185 of Hutcheson et al., which are expressly incorporated by reference herein.

### Ultrasonic Spray

The chemical means may be a source of active material from the group consisting of describes an ultrasonic spray coating system comprising an ultrasonic transducer with spray forming head, integrated fluid delivery device with air and liquid supply passage ways, support brackets and an ultrasonic power generator. The ultrasonic transducer consists of an ultrasonic converter that converts high frequency electrical energy into high frequency mechanical energy. The converter has a resonant frequency. A spray forming head is coupled to the converter and is resonant at the same resonant frequency of the converter. The spray forming head has a spray-forming tip and concentrates the vibrations of the converter at the spray-forming tip. The separate passage ways for air and the liquid supply allows the dilute hypochlorite to remain separated from potential contaminants until used. The ultrasonic transducer can produce a fine mist or a spray as the tranducer is adjusted. Additional ultrasonic spray devices are described in U.S. Pat. App. 2004/0256482 to Linden and U.S. Pat. No. 6,651,650 to Yamamoto et al., which describes an ultrasonic atomizer for pumping up a liquid from a liquid vessel by an ultrasonic pump and atomizing the liquid by passing it through a mesh plate formed to have multiplicity of minute holes. The device can be controlled for automatic, manual, or intermittent operation. See U.S. Pat. Apps. 2003/0056648 directed to Fornai et al. and 2005/0035213 directed to Erickson et al.

### Nonwoven Substrate

In one embodiment, the substrate of the present invention is composed of nonwoven fibers or paper. The term nonwoven is to be defined according to the commonly known definition provided by the “Nonwoven Fabrics Handbook” published by the Association of the Nonwoven Fabric Industry.

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The air-laying process is described in U.S. Pat. App. 2003/0036741 to Abba et al. and U.S. Pat. App. 2003/0118825 to Melius et al. The resulting layer, regardless of its method of production or composition, is then subjected to at least one of several types of bonding operations to anchor the individual fibers together to form a self-sustaining substrate. In the present invention the nonwoven substrate can be prepared by a variety of processes including, but not limited to, air-entanglement, hydroentanglement, thermal bonding, and combinations of these processes. Additional descriptions of dilute hypochlorite and packaging technology are found in Co-pending U.S. Pat. App. 2005/0232848, entitled “Packaging for Dilute Hypochlorite.

In one aspect, dry cleaning substrates can be provided with dry or substantially dry cleaning or disinfecting agents coated on or in the multicomponent, multilobal fiber layer. In addition, the cleaning substrates can be provided in a pre-moistened and/or saturated condition. The wet cleaning substrates can be maintained over time in a sealable container such as, for example, within a bucket with an attachable lid, sealable plastic pouches or bags, canisters, jars, tubs and so forth. Desirably the wet, stacked cleaning substrates are maintained in a resealable container. The use of a resealable container is particularly desirable when using volatile liquid compositions since substantial amounts of liquid can evaporate while using the first substrates thereby leaving the remaining substrates with little or no liquid. Exemplary resealable containers and dispensers include, but are not limited to, those described in U.S. Pat. No. 4,171,047 to Doyle et al., thereof. The treatment may be provided by typical chemical compositions or cleaning substrates, for example, U.S. Pat. App. 2003/0228996 to Hei et al., U.S. Pat. No. 4,353,480 to McFadyen, U.S. Pat. No. 6,576,604 to Hoshino et al., U.S. Pat. No. 4,778,048 to Kaspar, U.S. Pat. No. 6,200,941 to Strandburg et al., U.S. Pat. No. 4,741,944 to Jackson et al., U.S. Pat. No. 5,972,864 to Counts, U.S. Pat. No. 5,595,786 to McBride et al.; the entire contents of each of the aforesaid references are incorporated herein by reference. The cleaning substrates can be incorporated or oriented in the container as desired and/or folded as desired in order to improve ease of use or removal as is known in the art. The cleaning substrates of the present invention can be provided in a kit form, wherein a plurality of cleaning substrates and a cleaning tool are provided in a single package. Suitable systems are described in U.S. Pat. No. 5,972,239 to Coyle-Rees, U.S. Pat. No. 5,929,013 to Kuriyama et al., U.S. Pat. No. 5,869,440 to Kobayashi et al., U.S. Pat. No. 5,783,550 to Kuriyama et al., U.S. Pat. App. 2004/0072712 to Man et al., U.S. Pat. No. 5,688,756 to Garabedian et al., U.S. Pat. No. 6,624,134 to Briatore et al., Co-pending Application Serial (Docket No. 340.182), which was filed March 23, 20042005/0221113, entitled “Packaging for Dilute Hypochlorite”; Co-pending Application U.S. Pat. App. 2005/0232847, entitled “Method for Diluting Hypochlorite”; and Co-pending Application U.S. Pat. App. 2005/0214386, entitled “Methods for deactivating allergens and preventing disease”, Co-pending application Ser. No. 10/632,573, which was filed Aug. 1, 2003, entitled “Disinfecting article with extended efficacy”, and incorporated herein.

### Cleaning Substrate

A wide variety of materials can be used as the cleaning substrate. The substrate should have sufficient wet strength, abrasivity, loft and porosity. Examples of suitable substrates include, nonwoven substrates, wovens substrates, hydroentangled substrates, foams and sponges. Any of these substrates may be water-insoluble, water-dispersible, or water-soluble. Suitable substrates are described in Co-pending application Ser. No. 10/882,001, which was filed Jun. 29, 2004, entitled “Cleaning Pad with Functional Properties”, and incorporated herein.

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The air-laying process is described in U.S. Pat. App. 2003/0036741 to Abba et al. and U.S. Pat. App. 2002/0193278 to Cermenati et al. Surface treatments have been developed for residual mold control, for example, PCT App. WO2002/064877 to Rohrbaugh et al., U.S. Pat. App. 2003/0171446 to Murrer et al., and U.S. Pat. App. 2002/0028288 to Rohrbaugh et al. Devices that have been developed for residual mold control include U.S. Pat. App. 2003/0032569 to Takemura et al. and U.S. Pat. No. 6,463,600 to Conway et al. 2003/0118825 to Melius et al. The resulting layer, regardless of its method of production or composition, is then subjected to at least one of several types of bonding operations to anchor the individual fibers together to form a self-sustaining substrate. In the present invention the nonwoven substrate can be prepared by a variety of processes including, but not limited to, air-entanglement, hydroentanglement, thermal bonding, and combinations of these processes.

### Method of Use

The composition may be dispersed into the air. The composition may be dispersed by using an atomizer, a vaporizer, a nebulizer, a hose with laser created slits, or a spray device. The composition may be delivered on a continuous basis, such as with a humidifier. The composition may be delivered on a pulsed basis, such as with a canister on a timer. One spray device is an electrostatic sprayer, as described in PCT App. WO01/20988. The composition may be applied to skin surfaces. The composition may be delivered from a variety of containers, such as a dual chambered bottle, a trigger spray bottle, an aerosol canister, and a bleach pen. The compositions may be contained within a treatment device.

The composition may be applied to soft surfaces including clothing, bedding, upholstery, curtains, and carpets. The composition may be applied to soft surfaces by spraying, by wiping, by direct application, by immersion, or as part of the laundry washing process.

The composition may be applied to hard surfaces including kitchen surfaces, bathroom surfaces, walls, floors, outdoor surfaces, automobiles, countertops, food contact surfaces, toys, food products including fruits and vegetables. The composition may be applied to hard surfaces by spraying, by wiping, by direct application, by immersion, or as part of the normal cleaning process.

The composition may be applied with a nonwoven substrate, wipe or cleaning pad on inanimate, household surfaces, including floors, counter tops, furniture, windows, walls, and automobiles. The composition may be applied to baby and children's items, including toys, bottles, pacifiers, etc. Other surfaces include stainless steel, chrome, and shower enclosures. The nonwoven substrate, wipe or cleaning pad can be packaged individually or together in canisters, tubs, etc. The nonwoven substrate, wipe or cleaning pad can be used with the hand, or as part of a cleaning implement attached to a tool or motorized tool, such as one having a handle. Examples of tools using a nonwoven substrate, wipe or pad include U.S. Pat. No. 6,611,986 to Seals, WO00/71012 to Belt et al., U.S. Pat. App. 2002/0129835 to Pieroni and Foley, and WO00/27271 to Policicchio et al.

For certain uses, the composition may be thickened. The composition may be thickened by surfactant thickening, polymer thickening, or other means. Thickening may allow more controlled application or application from a device. Examples of thickened and unthickened compositions can be found in U.S. Pat. No. 6,162,371, U.S. Pat. No. 6,066,614, U.S. Pat. No. 6,153,120, U.S. Pat. No. 6,037,318, U.S. Pat. No. 6,313,082, U.S. Pat. No. 5,688,435, U.S. Pat. No. 6,413,925, U.S. Pat. No. 6,297,209, U.S. Pat. No. 6,100,228, U.S. Pat. No. 5,916,859, U.S. Pat. No. 5,851,421, U.S. Pat. No. 5,688,756, U.S. Pat. No. 5,767,055, U.S. Pat. No. 5,055,219, and U.S. Pat. No. 5,075,029.

The composition may be prepared by mixing a solid composition with water. The solid composition may be a tablet, granular composition, paste, or other solid composition. The composition may be prepared by diluting a liquid composition with water. The water may be purified. The composition may be prepared by mixing two liquids, for example, from a dual chambered container or a dual chambered spray bottle. The compositions of the invention can be delivered as part of a multi-compartment delivery system, for example as described in U.S. Pat. No. 5,954,213, U.S. Pat. No. 5,316,159, WO2004/014760, U.S. Pat. No. 6,610,254, and U.S. Pat. No. 6,550,694.

The composition may be part of an article of manufacture, wherein said article of manufacture in addition to the usage instructions bears an additional indication comprising a term selected from the group consisting of: neutralizes mold allergens, denatures toxins from mold, neutralizes toxins from mold, neutralizes protein allergens, controls allergens, removes allergens by cleaning, removes allergens by wiping, removes allergens in the laundry, reduces respiratory illness, reduces hay fever, reduces absenteeism, denatures mold allergens, prevents allergenic reactions, prevents allergenic reaction in humans, prevents allergenic symptoms due to mold, kills mold, destroys mold spores, destroys mold spores that cause adverse health effects, proven to prevent mold-triggered allergic sensitization in humans, proven to prevent mold-triggered allergic sensitization in animals, reduces the risk of mold-triggered allergic sensitization, reduces the risk of mold-triggered allergic response, destroys mold spores that induce allergic symptoms, neutralizes mold specific antigens, and prevents non-immune inflammatory reactions to mold.

The article of manufacture may include a set of instructions. The set of instructions may be used with a method of instructing the public by providing to the public a set of instructions for the use of an article of manufacture. The method of instructing the public may include information that an allergic response represents a response to pollen, dust mite, or mold allergens. The set of instructions may be provided to the public via electronic and/or print media. The set of instructions may be posted at the point of sale adjacent the package. The set of instructions may be posted on a global computer network at an address associated with products from a group consisting of said liquid composition, said target surface, or a combination thereof.

The following patents are incorporated herein by reference for their disclosure related to nonwovens: U.S. Pat. No. 3,862,472; U.S. Pat. No. 3,982,302; U.S. Pat. No. 4,004,323; U.S. Pat. No. 4,057,669; U.S. Pat. No. 4,097,965; U.S. Pat. No. 4,176,427; U.S. Pat. No. 4,130,915; U.S. Pat. No. 4,135,024; U.S. Pat. No. 4,189,896; U.S. Pat. No. 4,207,367; U.S. Pat. No. 4,296,161; U.S. Pat. No. 4,309,469; U.S. Pat. No. 4,682,942; U.S. Pat. No. 4,637,859; U.S. Pat. No. 5,223,096; U.S. Pat. No. 5,240,562; U.S. Pat. No. 5,556,509; and U.S. Pat. No. 5,580,423.

The compositions may be used in personal care applications, including uses to treat wounds, rashes, acne, etc. Example of suitable uses include: sprinkling on wound before bandaging, treatment for urishol-induced rashes (e.g. poison ivy, poison oak), as a band-aid additive, as a wound cleaner and disinfectant, as a treatment for athlete's foot fungus, as a facial anti-acne defoliator, as a diaper rash preventer, as an acne facial wash powder, or suspended as particles in a cream or other carrier.

Other suitable personal care uses might include: a denture cleaner; a hand sanitizer/moisturizer, as a waterless hand sanitizer, as a anti-gingivitis toothpaste, as a tooth whitener including good for gums claim, as a foot powder deodorizer, as a mouth freshener, as a portable dry shower or deodorant, as a skin lightener for “age spots”, as a hand sanitizer and moisturizer. Other potential uses include treating odors caused by bacteria and mildew, as a shoe cleaner, gym disinfecting powder, as a diaper pail odor remover, as a fridge deodorizer/freshener, as a sachet placed in food container, as sachet drawer fresheners, shoe powder deodorizer, as an air freshener for cars, as a garbage deodorizer, as a laundry dryer clothes freshener, as a garbage disposal freshener, for use anywhere baking soda is used, in a kitty litter box, as a freshener to carpets. Other potential uses include as a travel sanitizer, including camping gear, to treat cutting boards, as a powder to drop into air ducts to clean air, for waterless baby toy disinfecting, for closet mildew prevention, and as a seed treatment. Other potential uses include for water treatment, including as an additive for swimming pools, for cut flower freshness, for use in water filters for removal of microorganisms, and for direct addition to water. Other potential uses include use as a sprayable cleaning product, as a laundry detergent with bleach, to improve the odor control of an existing product, as a dry disinfecting wipe, in a direct bleach applicator device, as a dog/cat pet wash to treat odors, allergens, and as a disinfectant, as an upholstery cleaner to treat allergens, odors, germs, for waterless dish washing, as an additive to diapers to prevent odors or disinfect. Other potential uses include incorporation into items for long term use, for example in a sponge treatment so that sponge releases bleach with use, as an anti-mold building material additive, as an additive for grout and caulking, and as an additive to air filters for antimicrobial efficacy. Other potential uses include use to treat pests, for example as an ant preventer or for garden dusting. Other potential uses include industrial uses, including contaminated spill clean-up, algae removal from drinking water containers for farming, treating sick building syndrome, and as a general purpose disinfectant for hospitals. Other potential uses are in allergen deactivation (i.e. reaction of hypochlorous acid vapor to destroy proteins) and Weapon of Mass Destruction deactivation (e.g. hypochlorous acid vapor destroys many chemical weapons as well as microbial agents). Hypochlorous acid vapors can also deactivate many toxic gases such as cyanide, and hypochlorous acid vapor can also deactivate bacterial toxins—this could be useful where ever food is handled or served, could be useful for home canning—an alternate way to sterilize canning jars using hypochlorous acid vapors instead of boiling water, etc. Laponite® clay shear thins. The shear thinning behavior is suitable for dispensing through a spray applicator that may be trigger or pump activated or an aerosol. It then rethickens on the surface.

The clay materials can be described as expandable layered clays, i.e., aluminosilicates and magnesium silicates. The term “expandable” as used to describe the instant clays relates to the ability of the layered clay structure to be swollen, or expanded, on contact with water. The expandable clays used herein are those materials classified geologically as smectites (or montmorillonite) and attapulgites (or polygorskites). Smectites are three-layered clays. There are two distinct classes of smectite-type clays. In the first, aluminum oxide is present in the silicate crystal lattice; in the second class of smectites, magnesium oxide is present in the silicate crystal lattice. The general formulas of these smectites are Al₂(Si₂O₅)₂(OH)₂ and Mg₃(Si₂O₅)(OH)₂, for the aluminum and magnesium oxide type clays, respectively. It is to be recognized that the range of the water of hydration in the above formulas may vary with the processing to which the clay has been subjected.

Commercially available clays include, for example, montmorillonite (bentonite), volchonskoite, nontronite, beidellite, hectorite, saponite, sauconite and vermiculite. The clays herein are available under various trade names such as Gelwhite H NF® and Gelwhite GP® from Southern Clay Products. (both montmorillonites); Van Gel O® from R. T. Vanderbilt, smectites, laponites and layered silicates from Southern Clay Products. A second type of expandable clay material useful in the instant invention is classified geologically as attapulgite (polygorskite). Attapulgites are magnesium-rich clays having principles of superposition of tetrahedral and octahedral unit cell elements different from the smectites. Like the smectites, attapulgite clays are commercially available. For example, such clays are marketed under the tradename Attagel®, i.e. Attagel 40®, Attagel 50® and Attagel 150® from Engelhard Minerals & Chemicals Corporation.

One such synthetic mineral is sodium lithium magnesium silicate (CAS Reg. No. 53320-86-8) and in the Cosmetic, Toiletries and Fragrance Association (CTFA) dictionary as Sodium Magnesium Silicate. This synthetic mineral is sold commercially under the trade name Laponite®, a registered trademark of Southern Clay Products, Inc., Gonzales, Tex.

The thickener may form a viscous solution, a flowable gel or a rigid gel. The thickener component may be used in amounts of about 0.1% to 10% by weight.

### Application

The composition may be stored or shipped, or applied in a variety of containers, container materials, including glass, ABS, polycarbonate, high density polyethylene, low density polyethylene, high density polypropylene, low density polypropylene, polyethylene terephthalate, or polyvinylchloride. A variety of additives may affect the stability of the composition. For instance, the density of the polyethylene resin may be modified by co-polymerizing with a small amount of a short chain alkylene, e.g., butene, hexene or octene. Various other additives can be added, such as colorants, UV blockers, opacifying agents, and antioxidants, such as hindered phenols, e.g., BHT, Irganox 1010 (Ciba-Geigy A.G.), Irganox 1076 (Ciba-Geigy A.G.), Tonol (Shell Chemical Co.). Mold release agents and plasticizers can be added, especially to other types of plastics. The containers may have barrier films to increase storage stability. Suitable barrier films may include nylons, polyethylene terephthalate, fluorinated polyethylenes, and Barex (a copolymer of acrylonitrile and methylmethacrylate that is available from British Petroleum). The composition may be dispersed into the air. The composition may be dispersed into air using an aerosol or an electrostatic sprayer, as described in WO01/20988. The composition can be applied by the various device described in U.S. Pat. App. File number 340.182C, filed Mar. 31, 2005 to Bitowft et al.

The composition may be applied to soft surfaces including clothing, bedding, upholstery, curtains, and carpets. The composition may be applied to soft surfaces by spraying, by wiping, or by direct application, by immersion, or as part of the laundry washing process.

The composition may be applied to hard surfaces including kitchen surfaces, bathroom surfaces, walls, floors, outdoor surfaces, automobiles, countertops, food contact surfaces, toys, food products including fruits and vegetables. The composition may be applied to hard surfaces by spraying, by wiping, or by direct application, by immersion, or as part of the normal cleaning process.

The composition may be applied on human and animal surfaces, including external skin areas and internal cavities. The composition may have low skin sensitivity and may be appropriate to be taken orally or by inhalation. The composition may be applied to human and animal surfaces by spraying, by wiping, by direct application, by immersion, or as part of the normal treatment process. The composition may be applied as a thickened gel. The composition may be applied using a device to direct its application, such as a bleach pen. The composition may be applied as a wound dressing.

The composition may be applied with a nonwoven substrate, wipe or cleaning pad on inanimate, household surfaces, including floors, counter tops, furniture, windows, walls, and automobiles. Other surfaces include stainless steel, chrome, and shower enclosures. The composition may be applied to baby and children's items, including toys, bottles, pacifiers, etc. The composition may be applied with a nonwoven substrate, brush, sponge, wipe or cleaning pad on human and animal surfaces, including external skin areas and internal cavities. Other surfaces include stainless steel, chrome, and shower enclosures. The nonwoven substrate, wipe or cleaning pad can be packaged individually or together in canisters, tubs, etc. The nonwoven substrate, wipe or cleaning pad can be used with the hand, or as part of a cleaning implement attached to a tool or motorized tool, such as one having a handle. Examples of tools using a nonwoven substrate, wipe or pad include U.S. Pat. No. 6,611,986 to Seals, WO00/71012 to Belt et al., U.S. Pat. App. 2002/0129835 to Pieroni and Foley, and WO00/27271 to Policicchio et al.

For certain uses, for example, for human and animal surfaces, the composition may be thickened. The composition may be thickened using surfactant thickening, polymer thickening, for example clays, or other means. Thickening may allow more controlled application or application from a device. The composition may be thickened to a viscosity of from 40 to 10,000 cps. Examples of thickened and unthickened compositions can be found in U.S. Pat. No. 6,162,371, U.S. Pat. No. 6,066,614, U.S. Pat. No. 6,153,120, U.S. Pat. No. 6,037,318, U.S. Pat. No. 6,313,082, U.S. Pat. No. 5,688,435, U.S. Pat. No. 6,413,925, U.S. Pat. No. 6,297,209, U.S. Pat. No. 6,100,228, U.S. Pat. No. 5,916,859, U.S. Pat. No. 5,851,421, U.S. Pat. No. 5,688,756, U.S. Pat. No. 5,767,055, U.S. Pat. No. 5,055,219, and U.S. Pat. No. 5,075,029.

The anodic oxidation of chloride in an electrolysis cell results in the production of a number of oxychlorine ions including hypochlorite, chlorite, chlorate, and perchlorate. This electrolysis product is often referred to as oxidized water. Chlorite is readily oxidized to chlorate. Perchlorate may be an undesirable contaminant in the environment due to its low reactivity, high mobility, and inhibition of thyroid function. The production of hypochlorite via chlorination of caustic water is not believed to result in the initial formation of perchlorate. This route may be advantageous for certain uses where minor amounts of perchlorate would be undesirable.

The composition may be prepared by mixing a solid composition with water. The solid composition may be a tablet, granular composition, paste, or other solid composition. The composition may be prepared by diluting a liquid composition with water. The water may be purified. The composition may be prepared by mixing two liquids, for example, from a dual chambered container or a dual chambered spray bottle.

### Cleaners

The compositions of the invention can be diluted prior to use with tap water or water of higher purity. Preparation of dilute compositions for storage, for example as pre-diluted in bottles, may require water of higher purity. This higher purity water can be obtained by a variety of processes, including for example, distillation, filtering, sodium cation exchange (soft water), hydrogen cation exchange (deionized water without anion exchange), reverse osmosis, activated carbon treatment, ultrafiltration, nanofiltration, electrodialysis, and UV light treatment.

The compositions may be used in a direct application, sprayable or aerosolized product on hard surfaces, for cleaning, odor control, bleaching and sanitization. The compositions of the invention can be diluted prior to use from a concentrated liquid or solid composition. For instance, liquid sodium hypochlorite optionally containing surfactants combined with other dry ingredient cleaners, for example, laundry detergents or other additives of 5.25% available chlorine concentration (or above 0.5% concentration) can be diluted to below 500 ppm available chlorine concentration (or below 200 ppm). Tablets or powders having solid hypochlorite or hypochlorite generators can be dissolved in water to deliver compositions below 500 ppm concentration. Examples of compositions that can be diluted are described in U.S. Pat. No. 6,297,209, U.S. Pat. No. 6,100,228, U.S. Pat. No. 5,851,421, U.S. Pat. No. 5,688,756, U.S. Pat. No. 5,376,297, U.S. Pat. No. 5,034,150, U.S. Pat. No. 6,534,465, U.S. Pat. No. 6,503,877, U.S. Pat. No. 6,416,687, U.S. Pat. No. 6,180,583, and U.S. Pat. No. 6,051,676 for abrasive cleansers. The compositions can be applied to a woven or nonwoven substrate and used as a dry disinfecting wipe, for odor control, as an additive to diapers, for waterless dishwashing, for touching up fabric and upholstery. The method provides a safe and easy way to sanitize hard-to reach and difficult to sanitize objects and locations using dry, airborne technology. The method allows consumers to easily sanitize objects that they know have germs, but in a variety of forms including a pouch, sachet, a stick-up, a flat disc, and a powder dispenser. The pouch or other form can be vapor permeable, such as Tyvec® (HDPE) pouches. Semi-permeable films, membranes or nonwovens that allow hypochlorous acid vapors to largely permeate, but restrict the release of water vapors may also be used (e.g. Goretex® films). The method can be used in a variety of spaces, including toy boxes, closets, laundry hampers, trash cans and diaper pails, behind the toilet, and under the kitchen sink. The method can be used for batch sanitizing, preventing odors, preventing mold and mildew growth, sanitizing objects with “nooks and crannies”. Products using this technology both kill germs (Staph) and inhibit mold growth in an enclosed space. The composition can be used in a method of controlling odors, for example general cooking odors, bathroom or refrigerator odors, or odors from biofilm. The compositions can also control the growth of biofilm. One particular consumer problem is the growth of biofilm in washing machines or dryers, such as the new high efficiency washing machines. These compositions could be used to prevent or control the growth of biofilm in washing machines or control odors from that biofilm. For example a sachet or package containing the composition might be tossed into the washer or attached or otherwise connected to the inside of the washer. Since the composition can be effective by delivering the oxidant vapor, the composition can be effective in a hard to reach area of a laundry appliance.

The composition can be used in a method of controlling the growth of mold or bacteria using the steps of placing at least one particle in a confined space, allowing hypochlorous vapor from the particle to contact the mold or bacteria, wherein the growth of mold or bacteria is controlled or eliminated. The composition can be used in a method deactivating allergens using the steps of placing at least one particle in a confined space, allowing hypochlorous acid vapor from the particle to contact the allergen or allergen generating species, wherein the allergen is deactivated.

The compositions of the invention can be delivered as part of a multi-compartment delivery system, for example as described in U.S. Pat. No. 5,954,213, U.S. Pat. No. 5,316,159, WO2004/014760, U.S. Pat. No. 6,610,254, and U.S. Pat. No. 6,550,694.

### Allergen Deactivation

During the course of evaluating various oxidants and antimicrobials for their allergen deactivating ability, we have found that a very dilute solution (on the order of 40-80 ppm) of primarily hypochlorous acid can effectively deactivate allergens. Presumably the low levels of oxidant are still able to break up the allergen proteins, rendering them biologically inert.

While still extremely effective, the low concentration and nearly neutral pH (5-8) of the hypochlorous/hypochlorite mixture virtually eliminates surface damage. There is no sticky residue that can affect the feel of fabrics and there may also be minimal dye damage. The solution may be aerosolized to treat air directly, or applied to surfaces effective.

Aerosols are known to have a low collision rate between denaturant and allergen particles. As a result, the denaturant must be used in high concentrations to be effective. Using this approach with conventional denaturants, which may be irritating or fragranced at high levels, can cause health problems.

Dust mites, house dust, animal dander, animal hair, and the like, represent a mix of substances that contain allergens. Not all substances found in dust mite, house dust, animal dander, animal hair, etc. are capable of inducing an immune response, much less an allergic response. Some of these substances are antigens. They will induce a specific immune response. Some of these antigens are also allergens—they will induce a hypersensitivity response in susceptible individuals. Common allergens present indoors include, but are not limited to, Dermarophagoides pteronyssinus and Dermatophagoides farinae (both from dust mites), Felis domesticus (from cats), Canis familiaris (from dogs), Blatella germanica (from German cockroach), Penicillium, Aspergillus and Cladosporium (from fungi), as well as allergens from outdoors that enter the indoor environment, eg., pollen allergens.

As used herein, the term “allergen” refers to “the ability of certain materials to induce specific manifestations of hypersensitivity in man . . . and the associated special antibodies in the serum of such patients are known as reagins.” K. Landsteiner, THE SPECIFICITY OF SEROLOGICAL REACTIONS 9 (Dover Publications, NY, rev. ed. 1962), which is hereby incorporated by reference. A reagin is defined as an antibody found in the blood of individuals having a genetic predisposition to allergies. Allergy is the study and treatment of human hypersensitivity reactions producing a pathogenic response to nonself molecules termed allergens. Hypersensitivity (allergic) responses are a type of immune response. Antigens that induce hypersensitivity responses are known as allergens.

As used herein, the term “allergy-related product” refers to products that are marketed to help relieve and/or prevent allergy-related symptoms or control allergens, as well as the source of allergens, such as dust mites. Allergy-related products include, but are not limited to: non-prescription drugs; prescription drugs, especially including, but not limited to, antihistamines, antiinflammatory drugs, glucocorticosteroids, beta-adrenergics and leukotriene modifiers or antagonists; products that control and/or kill the sources of allergens, such as dust mites, including, but not limited to, carpet powders, household sprays, pillowcases, and mattress covers; air filters; HEPA filters; vacuums, especially those with HEPA filters; air purification devices; air pollution monitors; books (especially those relating to the treatment of allergy-related symptoms); face masks for filtering air; water filters (especially those for use in showers and/or bathtubs); household cleaning products, including, but not limited to, hard surface cleaning detergents (especially for floors and countertops), dusting sprays (especially for dusting and/or polishing furniture and household surfaces), and laundry detergents and/or additives capable of controlling and/or killing allergens and the sources thereof, personal cleansing products for either humans and/or animals including, but not limited to, bar soaps, liquid soaps, shampoos, and skin lotions; and the like. As defined herein, the term “allergy-related product” further includes the present cleaning sheets, implements, and articles of manufacture.

In one embodiment, the products can be used on food preparation surfaces and can contain only food-safe ingredients. Compositions for use herein may contain only materials that are food grade or GRAS, including, of course, direct food additives affirmed as GRAS, to protect against possible misuse by the consumer. Failure to rinse thoroughly after cleaning is less of a concern if all of the ingredients are GRAS and/or food grade. In the United States of America, the use and selection of cleaning ingredients for the purpose of washing fruits and vegetables is described by the United States Code of Federal Regulations, Title 21, Section 173. 315: “Ingredients for use in washing or lye peeling of fruits and vegetables”. These regulations restrict the ingredients that can be used for direct contact with food to those described as “generally regarded as safe” (GRAS), and a few other selected ingredients. These sections also provide certain limitations on the amount of material that can be used in a given context.

In one embodiment, the present invention encompasses the method of spraying an effective amount of the composition for reducing malodor onto household surfaces. The composition may reduce malodors by chemically destroying or breaking down the malodor or cause of the malodor. The household surfaces can be selected from the group consisting of countertops, cabinets, walls, floors, bathroom surfaces and kitchen surfaces. Other suitable household surfaces include pet areas, pet litter, litter boxes, pet bowls, and pets. The present invention encompasses the method of spraying a mist of an effective amount of the composition for reducing malodor onto fabric and/or fabric articles. The fabric and/or fabric articles can include, but are not limited to, clothes, curtains, drapes, upholstered furniture, carpeting, bed linens, bath linens, tablecloths, sleeping bags, tents, car interior, e.g., car carpet, fabric car seats, etc. The present invention relates to the method of spraying a mist of an effective amount of the composition for reducing malodor impression into the air to absorb malodor. The present invention relates to the method of spraying a mist of an effective amount of the composition for reducing malodor impression onto cat litter, pet bedding and pet houses to absorb malodor. The present invention relates to the method of spraying a mist of an effective amount of the composition for reducing malodor impression onto household pets to absorb malodor.

During the course of evaluating various oxidants and antimicrobials for their allergen deactivating ability, we have found that a very dilute solution (on the order of 40-200 ppm or more preferably 40-80 ppm) containing a substantial amount of hypochlorous acid can effectively deactivate allergens. Presumably the low levels of oxidant are still able to break up the allergen proteins, rendering them biologically inert.

While still extremely biocidally effective, the low concentration and nearly neutral pH (6.9) of hypochlorous acid virtually eliminates surface damage. There is no sticky residue that can affect the feel of fabrics and there may be minimal dye damage. The solution may be delievered to treat air directly, or applied to surfaces.

Aerosols Denaturant sprays and aerosol are known to have a low collision rate between denaturant and allergen particles. As a result, the denaturant must be used in high concentrations to be effective. Using this approach with conventional allergen denaturants, which may be irritating or fragranced at high levels, can cause health problems. Dilute hypochlorite compositions can have low irritancy and may be suitable to inactivate allergens and other contaminants in the air.

### Complete Mold System

The mold system can contain a combination of elements including: a mold detection device for collecting and analyzing mold presence in the home; detailed guidelines for how to take care of the mold problem based on results of the detection; components for removing or treating mold; components for ongoing mold prevention; and educational material about mold. The mold system might be part of a home construction kit targeting the bathroom. The mold system might be part of educational materials on how to maintain your home. The mold system might be part of a larger enterprise and could be expanded or broadened based on potential partnerships with (but not limited to) home insurers, property managers, professional mold remediation companies, health insurers, pharmaceutical companies, health-industry agencies (e.g. allergy associations) and government agencies (e.g. EPA, CA IAQ). The mold system may be provided in a satellite shop at the location selected from the group consisting of substantially within an existing retail store, substantially adjacent to an existing retail store, and a combination thereof.

### Mold Detection Device

The mold system can contain a mold detection device for collecting and analyzing mold presence in the home. The detection device may perform quantitative and qualitative testing. For example, the detection device may verify the presence of mold, the type and level of mold present. Examples of suitable detection devices include PCT App. WO03/031562 to Green et al., PCT App. WO2004/029216 to Han et al., U.S. Pat. No. 6,713,298 to McDevitt et al., U.S. Pat. No. 5,827,748 to Golden, U.S. Pat. No. 5,858,804 to Zanzucchi et al., U.S. Pat. No. 6,146,593 to Pinkel et al., U.S. Pat. No. 5,994,149 to Robinson et al., U.S. Pat. No. 6,729,196 to Moler et al., U.S. Pat. No. 6,303,316 to Kiel et al., and U.S. Pat. No. 6,192,168 to Feldstein et al., each incorporated herein by reference in their entirety.

The detection device may be based on a biosensor. A requirement for the biosensor may be that it is capable of detecting binding of an analyte to each binding moiety spot. The detection device may perform the analysis of a fluid containing one or more analytes. The device may be used for either liquid or gaseous fluids.

A biosensor is an apparatus that uses specific and/or selective binding interactions with one or more biomolecules (“ligands”), such as peptides, proteins, enzymes, antibodies, receptors, nucleic acids, aptamers, etc. to detect one or more target molecules (“analytes”). Binding of the target molecule to the ligand results in a signal that can be used to detect or quantify the analyte present in a sample. A wide variety of biosensors of different design are known. Typically, these are designed for use in clinical or research laboratories and tend to be very bulky and relatively fragile. For example, U.S. Pat. No. 6,258,606 discloses a multiplexed active biologic electrode array, allowing a variety of protein or nucleic acid biomolecules to be attached to specific locations on an integrated circuit chip. The biomolecules are exposed to samples and binding of various analyses to specific locations on the chip may be detected, for example, by fluorescence spectroscopy. U.S. Pat. No. 6,294,392 discloses a flow-through microchannel (capillary) biosensor that is said to be suitable for the detection of multiple different analyses in a sample by binding to complementary biomolecules immobilized on the wall of the microchannel. Following initial binding, immobilized complexes are denatured and flow past a downstream detector. U.S. Pat. No. 6,171,238 discloses a portable hand-held biosensor device for examination of whole blood, urine and other biological liquids. The system contains a single measuring electrode that can be covered by a biodiaphragm, limiting detection to single analyses at a time. U.S. Pat. No. 6,192,168 discloses a multimode waveguide device and fluidics cube apparatus that may be used as a biosensor. The waveguide may be attached to different biomolecules for detecting various analyses and may contain multiple channels for processing more than one sample at a time.

The biosensor of the detection device recognizes analytes meaning any compound, molecule or aggregate of interest for detection using the biosensor. Non-limiting examples of analyses include a protein, peptide, carbohydrate, polysaccharide, lipid, hormone, growth factor, cytokine, receptor, antigen, allergen, antibody, substrate, metabolite, cofactor, inhibitor, drug, pharmaceutical, nutrient, toxin, poison, explosive, pesticide, chemical warfare agent, biowarfare agent, biohazardous agent, infectious agent, prion, radioisotope, vitamin, heterocyclic aromatic compound, carcinogen, mutagen, narcotic, amphetamine, barbiturate, hallucinogen, waste product, contaminant, heavy metal or any other molecule or atom, without limitation as to size. Analytes are not limited to single molecules or atoms, but may also comprise complex aggregates, such as a virus, bacterium, Salmonella, Streptococcus, Legionella, E. coli, Giardia, Cryptosporidium, Rickettsia, spore, mold, yeast, algae, amoebae, dinoflagellate, unicellular organism, pathogen or cell. In certain embodiments, cells exhibiting a particular characteristic or disease state, such as a cancer cell, may be target analytes. Virtually any chemical or biological compound, molecule or aggregate could be a target analyte.

In various embodiments, the present invention concerns the use of binding moieties for the detection of analytes. Although in preferred embodiments the binding moieties are antibodies, it is contemplated within the scope of the invention that virtually any molecule or aggregate that can bind to a target analyte with sufficient affinity and specificity to allow its detection may be used. Non-antibody binding moieties that may be used within the scope of the present invention include, for example, aptamers (e.g., U.S. Pat. No. 5,843,653 to Gold et al.), peptide libraries (e.g., U.S. Pat. No. 6,068,829 to Ruoslahti et al., incorporated herein by reference), and various receptor proteins, binding proteins, cell surface proteins, and other non-antibody peptides or proteins known in the art.

The terms “detection” and “detecting” are used herein to refer to an assay or procedure that is indicative of the presence of one or more specific analytes in a sample, or that predicts a disease state or a medical or environmental condition associated with the presence of one or more specific analyses in a sample. It will be appreciated by those of skill in the art that all assays exhibit a certain level of false positives and false negatives. Even where a positive result in an assay is not invariably associated with the presence of a target analyte, the result is of use as it indicates the need for more careful monitoring of an individual, a population, or an environmental site. An assay is diagnostic of a disease state or a medical or environmental condition when the assay results show a statistically significant association or correlation with the ultimate manifestation of the disease or condition.

The specimen might be sent for analysis to an offsite laboratory. The results of the test and/or the treatment guidelines might be provided over a computer communications network such as the internet. The application device can be an aerosol or non-aerosol device. The product can be sprayed using any suitable type of sprayer. One suitable type of sprayer is an aerosol sprayer using a propellant. If an aerosol sprayer is used, it can use any suitable type of propellant. The propellant can include hydrocarbon propellants, or non-hydrocarbon propellants. A non-hydrocarbon propellant may include, but is not limited to a compressed gas. Suitable compressed gases include, but are not limited to compressed air, nitrogen, inert gases, carbon dioxide, etc.

### Mold Treatment Guidelines

The mold system can contain detailed guidelines for how to take care of the mold problem based on results of the detection kit. For example, if the type and level of mold present is below a certain hurdle, the consumer might be directed to remove the mold using additional tools in the system. Step-by-step instructions would guide the consumer on how to remove the mold. If the type and level of mold present is above a certain threshold, the consumer might be referred to a professional mold remediation company, who might have a relationship with the mold system provider. The method of providing test results and/or treatment guidelines might include a step of providing a Web page that is adapted to allow a person to enter the unique code onto the Web page and transmit an electronic message containing the unique code from a first computer communication network access device remotely-located from the off-site laboratory over the computer communications network to a second computer communication network access device located at the off-site laboratory. The computer located at the off-site laboratory can receive the electronic message containing the unique code and respond by transmitting an electronic message containing the test results over the computer communications network to the first computer communication network access device. See U.S. Pat. App. 2003/0052194 to Streutker et al., and U.S. Pat. No. 6,502,766 to Streutker et al.

The mold system can contain detailed guidelines for evaluating buildings for mold growth. Such instructions might include: “Check building materials and spaces for visible mold and signs of moisture damage indicating a history of water leaks and, including glass, ABS, polycarbonate, high humidity and condensation levels. Building ventilation systems should also be inspected. Basic precautions should be taken when investigating and evaluating mold and moisture problems. Such precautions could include: Do not touch mold or moldy items with bare hands; Do not get mold or mold spores in your eyes; Do not breathe mold or mold spores; Use personal protective equipment (PPE). At a minimum, use an N-95 NIOSH-approved respirator, gloves, and eye protection; and Contain or bag debris.”

Sampling instructions might include: “Sampling is usually not necessary when visible signs of mold growth are present. However, the American Industrial Hygiene Association (AIHA) indicates that in cases where health concerns are an issue, litigation is involved, or the source(s) of contamination is unclear, sampling may be considered. Professionals experienced with mold issues and familiar with current guidelines should conduct sampling and interpret results, as no threshold or exposure limits have been established. As a general guideline, the types and concentrations of mold in indoor air samples should be similar to those found in the local outdoor air. Samples should be analyzed by a laboratory that participates in proficiency testing such as the Environmental Microbiology Proficiency Analytical Testing Program, EMPAT.”

Remediation instruction might include: “Mold remediation prevents further human exposure and damage to building materials and furnishings. You must clean up and remove mold contamination, not just kill the mold. Dead mold is still allergenic; some are potentially toxic. Mold gradually destroys what it grows on; to grow, it needs an organic substrate, moisture, and oxygen. If mold growth is not addressed promptly, materials may be damaged and cleaning cannot restore appearance or integrity. Mold can generally be removed from nonporous (hard) surfaces by wiping or scrubbing with water or water and detergent. The use of disinfectant chemicals (biocides), including chlorine bleach, is not recommended as a routine practice. Biocides are of limited use in mold remediation and are not a substitute for thorough cleaning. Mold-contaminated porous material such as damp insulation in ventilation systems, moldy ceiling tile, and mildewed carpet may need to be removed and discarded. Remediate means to fix a problem. The first step in mold remediation is to fix the water or humidity problem that contributed to mold growth. Thoroughly clean up mold and dry water-damaged areas, using appropriate cleaning and drying methods. Mold remediation requires some level of isolation of materials or containment and the use of appropriate personal protective equipment (PPE). Remediation decisions should be based on the scope of contamination, size of the area of growth, and potential for occupant exposure or building contamination in the absence of containment. Professional expertise and conservative methods may be needed when the chance of mold becoming airborne is high or mold-sensitive individuals are present.”

Cleanup methods might include: “Small—less than 10 sq. ft. Example:

Carpet and backing. Wet vacuum. Use high-efficiency particulate air (HEPA) vacuum when thoroughly dry. Medium—10-100 sq. ft. Example: Concrete or cinder block. Wet vacuum. Use HEPA vacuum when thoroughly dry. Large—greater than 100 sq. ft. Example: Drywall or gypsum. Use HEPA-vacuum after thoroughly dry. Remove and discard damaged material.”

### Components for Removing and Treating Mold

The mold system can contain components for removing mold.

Suitable components might include: disposable gloves to prevent physical contact of the skin with mold; a disposable mask to prevent inhalation of mold spores; a pre-moistened wipe with diluted bleach to remove, kill and denature mold; a traditional hypochlorite spray product to remove and kill mold; an aerosol spray product to remove and kill airborne and surface mold; and a calorimetric Indicator to confirm cleaning and disinfecting process is successful. The mold system can also contain such items as mold-resistant grout, a tool for applying grout, a tool for removing old grout. Additional equipment required might include a N-95 respirator, goggles/eye protection, disposable overalls, and a HEPA-filtered fan unit.

Various components can be included in the mold system for treating mold in the home. For example, U.S. 2004/0001777 to Hobson et al. describes evaporating a solution of acidified oxyhalogen species. The treatment may be provided by filters such an HEPA filters, for example, as described in U.S. Pat. as described in U.S. Pat. App. 2003/0150327 to Bolden. The treatment may be provided by electrostatic filters, for example, as described in U.S. Pat. No. 6,656,253 to Willey et al. The treatment may provide a variety of treatment mechanisms, for example, as described in U.S. Pat. App. 2004/0047776 to Thomsen. The treatment may provide a chemical means to decontaminate, for example, U.S. Pat. App. 2003/0056648 to Fornai et al. The chemical means may be a source of active material from the group consisting of hypohalous acid, hypohalous acid salt, hypohalous acid generating species, hypohalous acid salt generating species, and combinations thereof. The treatment may be provided by typical chemical compositions or cleaning substrates, for example, U.S. Pat. App. 2003/0228996 to Hei et al., U.S. Pat. No. 6,576,604 to Hoshino et al., U.S. Pat. No. 6,200,941 to Strandburg et al., U.S. Pat. No. 5,972,864 to Counts, U.S. Pat. No. 5,972,239 to Coyle-Rees, U.S. Pat. No. 5,929,013 to Kuriyama et al., U.S. Pat. No. 5,869,440 to Kobayashi et al., U.S. Pat. No. 5,783,550 to Kuriyama et al., U.S. Pat. App. 2004/0072712 to Man et al., U.S. Pat. No. 5,688,756 to Garabedian et al., U.S. Pat. No. 6,624,134 to Briatore et al., Co-pending application Ser. No. 10/806,522 (Docket No. 340.182), which was filed Mar. 23, 2004, entitled “Methods for deactivating allergens and preventing disease”, Co-pending application Ser. No. 10/632,573, which was filed Aug. 1, 2003, entitled “Disinfecting Article With Extended Efficacy”, and Co-pending application Ser. No. 10/828,571, which was filed Apr. 23, 2004, entitled “Method for Diluting Hypochlorite”.

Educational Materials about Mold

The system may provide educational material about mold, including but not limited to technical information and pictures of common household mold, health effects of exposure to mold, preventive measures, tips on cleaning the home and maintaining a “healthy home”.

An example of educational information about mold includes the following statements. Molds are usually not a problem indoors, unless mold spores land on a wet or damp spot and begin growing. Molds have the potential to cause health problems. Molds produce allergens (substances that can cause allergic reactions), irritants, and in some cases, potentially toxic substances (mycotoxins). Inhaling or touching mold or mold spores may cause allergic reactions in sensitive individuals. Allergic responses include hay fever-type symptoms, such as sneezing, runny nose, red eyes, and skin rash (dermatitis). Allergic reactions to mold are common. They can be immediate or delayed. Molds can also cause asthma attacks in people with asthma who are allergic to mold. In addition, mold exposure can irritate the eyes, skin, nose, throat, and lungs of both mold-allergic and non-allergic people. Molds can also produce organic toxins. These toxins include Aflatoxin B, Citrinin, Cyclosporin A, Deoxynivalenol, Emodin, Gliotoxin, Griseofulvin, Ochratoxin A, Patulin, Roridin A, Satratoxin H, Sterigmatocystin, T-2 toxin, Verrucarin A, and Endotoxins. Molds are living organisms containing protein, lipids and carbohydrates. Thus, treatments that are effective for some chemicals may not be effective for molds. The use of a chemical or biocide that kills organisms such as mold is not recommended as a routine practice during mold cleanup. Dead mold may still cause allergic reactions in people, so it is not enough to simply kill the mold, it must also be destroyed or removed.

The first step in the educational materials might allow consumers to identify where they composition may have a mold problem and gauge the magnitude of their problem. The educational materials might include where to look for mold; such as, “Mold grows on organic materials, such as paper, dirt, wood and soap scum. Mold grows on moist materials, so mold growth is likely in areas wet by water leaks, flooding, humidity levels above about 70 percent and condensation. Any flooded area that was not completely dried within about one day is likely to have mold growth. Walls need to be opened and rapidly dried to prevent mold growth. Any area that is stained from water should be examined for mold growth. Peeling paint may be an indication of wet walls. Moisture seeping through concrete walls and floors will cause moist conditions likely to cause mold growth on or in walls, carpeting and materials stored in the basement. Mold often grows under cabinets, behind base-boards, inside walls, in carpet padding and under vinyl wall coverings. An unvented clothes dryer creates a very humid, warm environment conducive to mold growth. Closets may have mold growth if clothing is damp or if there is a cool outside wall in the closet. Also, there is a chance mold might be growing behind furniture, particularly against an outside wall. Mold will not normally be found in furnace or air-conditioning ducts unless they were flooded because the heated or air-conditioned air is very dry. Moisture coming through a basement floor or wall may deposit a light-colored salt and other minerals that are sometimes thought to be mold. The deposits should quickly dissolve and disappear when wet with water if they are a salt.”

The educational materials might include directions for mold removal; such as, “Since people react to mold whether it is living or dead, the mold must be removed. Take steps to protect your health during mold removal. Use a mask or respirator that will filter out mold spores. Usually it will be designated as an N95, 3M #1860 or TC-21C particulate respirator. Wear eye protection, rubber gloves and clothing that can be immediately laundered. Dampen moldy materials before removal to minimize the number of airborne mold spores. Mold can be removed pH from 12 to completely remove mold from porous surfaces such as paper, Sheetrock (drywall) and carpet padding, so these materials should be removed and discarded. Scrubbing may not completely remove mold growth on structural wood, such as wall studs, so it may need to be removed by sanding. Wear personal protective gear and isolate the work area from the rest of the home. After the mold is removed, disinfect the area using a bleach and water solution or another disinfectant. The amount of bleach recommended per gallon of water varies considerably. A clean surface requires less bleach than a dirty surface. A solution of ¼ cup bleach to 1 gallon of water should be adequate for clean surfaces. The surface must remain wet for about 15 minutes to allow the solution to disinfect. Concentrations as high as 1½ cups of bleach per gallon of water are recommended for surfaces that could not be thoroughly cleaned. Provide adequate ventilation during disinfecting and wear rubber gloves. Finally, rinse the entire area with clean water, and then rapidly dry the surfaces. Use fans and dehumidifiers or natural ventilation that exchanges inside air with outside air.”

The educational materials might include directions for preventing mold growth; such as, “The moisture problem must be fixed to prevent future mold growth. Since there are some mold spores everywhere and since mold grows on any wet organic surface, the only way to prevent mold growth is to keep things dry.”

The mold educational materials could include government materials, such as EPA's pamphlet, “Mold Remediation in Schools and Commercial Buildings.” It provides clean-up methods and remediation techniques and discusses precautions and the impact of mold on HVAC systems. Its guidelines are based on total surface area contamination and potential for remediator and occupant exposure. The mold educational materials could include referral to internet websites for additional information, such as www.epa.gov/iaq/molds and www.osba.gov/SLTC/molds.

### Treatment for Inhibiting Future Mold Growth

Chemical treatments have been developed for residual mold control, for example, PCT App. No. WO02/28990 to McKechnie; U.S. Pat. No. 6,559,111 to Colurciello et al., and U.S. Pat. App. 2002/0193278 to Cermenati et al. Surface treatments have been developed for residual mold control, for example, PCT App. No. WO2002/064877 to Rohrbaugh et al., U.S. Pat. App. No. 2003/0171446 to Murrer et al., and U.S. Pat. App. No. 2002/0028288 to Rohrbaugh et al. Devices that have been developed for residual mold control include U.S. Pat. App. No. 2003/0032569 to Takemura et al. and U.S. Pat. No. 6,463,600 to Conway et al.

### Water Purification

The compositions of the invention can be used to purify water and make the water safe for consumption or recreational use. The compositions of the invention can be used for algae control. The compositions of the invention can be incorporated into water filters, for example, for use while camping or in disasters.

### Food and Food Contact Surfaces

The compositions of the invention can be used for a direct food rinse treatment, for cleaning food-contact surfaces, and for toxicologically safe cleaning. This may involve the use of additional food-safe ingredients, GRAS ingredients, or ingredients with low toxicological impact. Methods describing this use and possible compositions can be found in U.S. Pat. No. 6,455,086, U.S. Pat. No. 6,313,049, U.S. Pat. App. No. 2002/0132742, U.S. Pat. App. No. 2001/0014655, PCT App. No. WO99/00025, and U.S. Pat. App. No. 2002/0151452.

### Personal Care

The compositions of the invention can be used to sterilize medical instruments. Dilute hypochlorite will discolor or degrade tubing and other sensitive parts to less extent than concentrated hypochlorite. The compositions may be used in kidney dialysis machines or as an irrigating agent in endodontic treatment. The compositions of the invention can be used to kill tumor cells, affect tumor cell recognition and to induce apoptosis.

The compositions of the invention can be used in agricultural applications, for example, seed and seedling treatments, dormant sprays for fruit trees, stored grain treatments, dips or sprays for any post-harvest plant material and their containers, treatments for soil, either on the land or in containers, treatments for transportation and storage to market, treatments for transportation, storage, and display at market (retail or wholesale), treatments for import and export regulations, and treatments for preventing the accidental introduction of alien pest organisms. The compositions of the invention can be used for the meat, poultry, dairy, seafood, and aquaculture industries, for example, equipment treatments, living quarters treatments, dips or sprays for eggs and containers, dips or sprays for meat and containers, treatments for rendering operations, treatments for transportation and storage to market, treatments for transportation, storage, and display at market (retail or wholesale), treatments for import and export regulations, treatments for preventing alien pest organisms from crossing borders, treating disease on live animals (terrestrial or aquatic), including udder treatments, and dips or sprays for milking equipment, transfer lines, and containers. The compositions of the invention can be used for homeland security, for example, treatments for preventing the intentional introduction of alien pest organisms or deadly human or animal organisms.

### Plant Preservation

The compositions of the invention can be used to preserve and maintain the freshness of freshly cut flowers and other cut plants. The compositions of the invention can be used to prevent the build-up of microorganisms that contribute to the decaying of stems and abscission and scenesing of leaves and flowers. The compositions of the invention can be used to preserve and extend the shelf life of freshly cut fruits and vegetables such as cut melon, cantaloupe, strawberry, potatoes, etc. The compositions of the invention can be used to eradicate hepatitis virus A from fresh strawberries and other fruits and vegetables. The compositions of the invention can be used for in the sprout industry to treat seeds of various plants including alfalfa, wheat, barely and all other edible plants to control the spread of food-borne diseases such as Salmonella, E. coli, Campylobacter, etc. The compositions of the invention can be used in washing and treating shoes that have been moldy. The compositions of the invention can be used with sponges, cheese-cloth, paper towel and other non-woven articles to clean and remove and kill mold, bacteria and viruses from soft and hard surfaces. The compositions of the invention can be used to control mold in school. The compositions of the invention can be used as a spray or wipe product. The compositions of the invention can be used to control the spread of germs on hard surfaces in school. The compositions of the invention can be used to control the spread of hepatitis among jails. The compositions of the invention can be used in laundry to kill germs. The compositions of the invention can be used in long-term care centers and public gyms, where, for example, they can be applied as a spray or wipe product on hard surfaces to kill all germs that are transmitted to environmental surfaces via human activity. The compositions of the invention can be used in laundry to disinfect towels, and other articles that carry germs. The compositions of the invention can be used in public areas where, for example, they can be sprayed on a large scale in parks, streets, public places to control disease-causing agents such as SARS, calicivirus, enterovirus, FMD, and other viruses. The compositions of the invention can be used as wipes or spray to disinfect all environmental surfaces. The compositions of the invention can be used on ships and cruise ships where, for example, they can be used to control the spread of norwalk virus, calicivirus, and influenza virus. The compositions of the invention can be used to control cross contamination due to Salmonella and Campylobacter. The compositions of the invention can be used to protect from biological warfare where, for example, they can be used to spray on humans, (i.e., army personnel, medics, etc.) in case of potential presence of biological warfare agents such as Anthrax, BT, Sarin, Small Pox, and SARS, etc. The compositions of the invention can be used for disinfecting military vehicles, airplanes, and others. The compositions of the invention can be used to control the outbreak of infectious agents where, for example, they can be used to disinfect airplanes (inside and outside), trains, buses and all sort of transportation means to control the spread of pathogens. The compositions of the invention can be used to disinfect shoes (via a wipe or dipping or spraying) at airports and other ports of entry. The compositions of the invention can be used to control insects where, for example, they can be used as a spray to kill New Zealand Slug and other slugs or insects. The compositions of the invention can be used to kill fleas. The compositions of the invention can be used to control animal and insect pathogens where, for example, they can be used to control animal and bird viruses on hard surfaces and soft surfaces. Such viruses include SARS, bird flu virus, calicivirus, mad cow disease virus, parvovirus, feline viruses, etc. Also, they can be used to dip teats in to control various pathogens.

The composition may be part of an article of manufacture of a kit comprising: a container enclosing a liquid composition; and a set of instructions; and a liquid composition comprising an allergen neutralizing agent selected from a group consisting of a hypohalous acid, a hypohalous acid salt, and a combination thereof, wherein said set of instructions comprises instructions to contact targets selected from a group consisting of hard surfaces, soft surfaces, or air with said liquid composition in its neat or diluted form. The powder composition may be on a nonwoven substrate. The set of instructions can be for use on soft inanimate surfaces (such as fabrics), hard inanimate surfaces (such as counter-tops), air (such as to destroy odors, germs, or allergens). The instructions can also be to prevent allergic response, to prevent illness, or a combination thereof

The composition may be part of an article of manufacture wherein said article of manufacture in addition to the usage instructions bears an additional indication comprising a term selected from the group consisting of: healthy, healthier, reduce the occurrence of illness, control the spread of illness in the home, protect your family from illness, keep your home healthier, keep your family well, break the cycle of illness in the home, reduce the risk of common illnesses, and combinations thereof.

The composition may be part of an article of manufacture, wherein said article of manufacture in addition to the usage instructions bears an additional indication comprising a term selected from the group consisting of: neutralizes mold allergens, denatures toxins from mold, neutralizes toxins from mold, neutralizes protein allergens, controls allergens, removes allergens by cleaning, removes allergens by wiping, removes allergens in the laundry, reduces respiratory illness, reduces hay fever, reduces absenteeism, denatures mold allergens, prevents allergenic reactions, prevents allergenic reaction in humans, prevents allergenic symptoms due to mold, kills mold, destroys mold spores, destroys mold spores that cause adverse health effects, proven to prevent mold-triggered allergic sensitization in humans, proven to prevent mold-triggered allergic sensitization in animals, reduces the risk of mold-triggered allergic sensitization, reduces the risk of mold-triggered allergic response, destroys mold spores that induce allergic symptoms, neutralizes mold specific antigens, and prevents non-immune inflammatory reactions to mold.

The composition may be part of an article of manufacture. The article of manufacture may include a set of instructions. The set of instructions may be used with a method of instructing the public by providing to the public a set of instructions for the use of an article of manufacture comprising a container and a liquid composition comprising an allergen neutralizing agent selected from a group consisting of a hypohalous acid, a hypohalous acid salt, and a combination thereof; wherein said set of instructions comprises instructions to contact targets selected from a group consisting of hard surfaces, soft surfaces, or air with said liquid composition in its neat or diluted form to prevent allergic response, to prevent illness, or a combination thereof. The instructions may relate to preventing the spread of illness with a liquid composition comprising a hypohalous acid salt composition. The method of instructing the public may include information that an allergic response represents a response to pollen, dust mite, or mold allergens. The set of instructions may be provided to the public via electronic and/or print media. The set of instructions may be posted at the point of sale adjacent the package. The set of instructions may be posted on a global computer network at an address associated with products from a group consisting of said liquid composition, said target surface, or a combination thereof.

The method of promoting the use of the liquid composition comprising an allergen neutralizing agent selected from a group consisting of a hypohalous acid, a hypohalous acid salt, and a combination thereof may include use instructions to prevent allergic response and/or illness, the method comprising the step of informing the public that the treatment of targets selected from a group consisting of hard surfaces, soft surfaces, or air with said composition reduces and/or prevents allergic response and/or illness. The method of promoting the use of the composition may include the step of informing the consumer via electronic and/or print media.

The use of the composition may include an in vivo test method for testing allergic response in animals, wherein said test method comprises the subcutaneous injection of allergens treated with a composition selected from a group consisting of a hypohalous acid, a hypohalous acid salt, and a combination thereof.

While still extremely effective, the low concentration and nearly neutral pH (6.9) of hypochlorous virtually eliminates surface damage. There is no sticky residue that can affect the feel of fabrics and there may be minimal dye damage. The solution may be aerosolized to treat air directly, or applied to surfaces.

Aerosols are known to have a low collision rate between denaturant and allergen particles. As a result, the denaturant must be used in high concentrations to be effective. Using this approach with conventional denaturants, which may be irritating or fragranced at high levels, can cause health problems. The use of a humidifier to deliver dilute hypohalous acid may reduce these problems.

Although hypohalous acid and hypohalous acid salt compositions can be useful over the entire pH range of 2 to 13, some benefits, such as the mold control, may require pH less than about pH 10, or less than pH 9, or less than pH 8, or less than pH 7. The compositions can include buffer systems, such as carboxylic acids and their salts, for example acetic acid or succinic acid. Other useful buffer systems would include borates, bicarbonates, hydrogen phosphates, and mixed metal silicates.

The hypohalous acid and hypohalous acid salt can be formed from the neutralization of chlorine gas with caustic solution, during which an equimolar amount of halide is also formed. In electrolysis, halide is consumed and none is formed. Dilute hypohalous acid and salt technology is described in U.S. Pat. App. 2005/0214,386, U.S. Pat. App. No. 2005/0216,291, U.S. Pat. App. No. 2005/0232,847, U.S. Pat. App. No. 2005/0232,848, U.S. Pat. App. No. 2005/0221,113, U.S. Pat. App. No. 2005/0233,900 and U.S. Pat. App Ser. No. 11/277,642 entitled “Antimicrobial Product Combination”, all of which are incorporated by reference herein.

### Humidifiers

Humidifiers deliver moisture into indoor spaces. One type of humidifier is an ultrasonic humidifier. Ultrasonic humidifiers generally comprise a container filled with water, which is excited by a piezoelectric disc that vibrates at a high frequency and in turn causes a phase change in the water by means of cavitation. An air stream directed onto the water surface carries the mist into the room to be humidified. The major drawback of both porous medium humidifiers and ultrasonic humidifiers is that the water staying in the container is not heated to its boiling point as in the steam generator and, is therefore susceptible to the growth of microorganisms, which are subsequently carried by the air stream into the room where it may be ingested by people. By delivering moisture from a sanitizing solution, this drawback can be avoided.

Another type of humidifier is a warm-air humidifier. Warm-air humidifiers share the benefits of steam generators in that growth of microorganisms is forestalled by heating the water to its boiling point. Also, warm-air humidifiers avoid the drawback of hot steam entering the room, since in this type of humidifier the steam is carried into the room as a mist mixed with air, at a temperature to be selected by judiciously choosing the ratio of steam and air. A typical warm-air humidifier is described in U.S. Pat. No. 4,564,746. This humidifier includes a heated evaporation chamber, which is enclosed to prevent leakage or damage and a fan adapted for dispersing the generated steam into the room via a cabinet passageway. The evaporation chamber is mounted on tracks, which permits it to be slid out of its enclosure for cleaning and servicing. The heating element, which is operationally enclosed in the chamber, is attached to a cover, which is likewise movable out of the humidifier cabinet for cleaning and servicing. PCT App. No. WO9514190 describes a portable and personal-sized electric warm air humidifier.

The humidifier can have replaceable or disposable cartridges containing dilute hypohalous acid that are readily placed in the humidifier. The replaceable cartridges can also be generators of hypohalous acid. The replaceable cartridges can also deliver additional ingredients. The humidifier can contain a fan. The materials used to manufacture the water container and transducer housing are compatible with the hypohalous acid solution to allow an effective treatment of microorganisms. In one embodiment of the application, the humidifier generates a dilute hypohalous acid vapor, which includes but is not limited to mists, aerosols, and gas. The hypohalous acid can prevent musty odor that emanates from the humidifier, which may be caused by mold in the humidifier vapor. The hypohalous acid can be used in the treatment of mold, treatment of allergens, treatment of bacteria, treatment of viruses, and combinations thereof.

Plug-In Air Treatment with Optional Fan

Plug-in diffusers are described in U.S. Pat. Nos. 4,849,606, and 5,937,140, both of which are incorporated herein by reference. A plug-in device can be designed to continuously or periodically release a fine mist of dilute hypochlorite. The plug-in can also optionally contain a fan or additionally release a fragrance. The device can kill germs and remove allergens while being safe to use around kids, pets, and food.

### Self-Generating Steam Apparatus

The device can be a self-generating steam apparatus as described in U.S. Pat. No. 2005/0262757 to Wong et al. that contains a self-steaming (including, vaporizing) composition such that the vaporizer is portable, has its own energy source, and is not dependent upon an external source of energy for operation. In one embodiment, a sub-article comprising the composition is contained within the vaporizer article, such that upon activation the composition is self-steaming (including, self-vaporizing) for the benefit of the user. In one embodiment, the composition interacts with air to generate heat and water vapor containing dilute hypohalous acid. For example, the composition may be activated as follows: The article comprising the composition may include an oxygen impermeable plastic overwrap. A tear-tab or notch may be included on the overwrap for easy access by a user. Instructions may be included with the enclosure instructing a user to tear open the overwrap to remove the article comprising the self-steaming composition. This opening action immediately mixes oxygen contained in the ambient air with the composition to initiate the self-steaming process.

### Portable Devices

The device can contain an energy source, such as batteries, and can also contain a means for allowing recharging of rechargeable internal batteries via such means as a plug or port such that the consumer can conveniently recharge the batteries. Other means of providing energy sources that allow the device to be portable include methanol fuel cells or minerals that generate heat upon mixture with water, for example, mixing water with anhydrous calcium oxide. Portable devices would allow for disposable humidifiers that could be taken for on the go occasions. For example, such systems could fit in the cup holders of vehicles.

### Dispersion Devices

In order to speed the distribution of the hypochlorous acid vapors various mechanical dispersing devices such as fans 64 (FIG. 6), piezoelectric sprayers, and ultrasonic dispersers may be used. The life time of the hypochlorous acid emission may be controlled by the surface area through which vapors are emitted relative to the amount of liquid or solid that contains hypochlorous acid. In addition to aqueous solutions made from sodium hypochlorite, solid N-chloro compounds may also be used, since these may react with humidity or moisture to emit hypochlorous acid.

In one embodiment as shown in FIG. 7, the smectites form an alkaline dispersion device is an air deodorizing device 71 having an air flow path from an air inlet 72 to an air outlet 73, and the deodorizing device 71 having a cartridge member 74 detachable from a portion of said deodorizing device 71, said cartridge member 74 comprising a filter member 75, wherein said cartridge member 74 is adapted to be arranged with respect to said portion of the deodorizing device such that said filter member 75 comes into contact with the air flowing along said air flow path of said deodorizing device 71; and an air moving member 76 for moving air along said air flow path, the air moving member 76 having a fan 77 connected to an electric motor (not shown) wherein said electric motor is powered by a source of electricity and wherein said air moving member is adapted to displace at least 10 ml or 100 ml of air per second through the air inlet of said deodorizing device.

### Electrolytically Generated Hypohalous Acid

The device may be a self-generating plug-in or portable device, for example as described in U.S. Pat. App. No. 2003/0213704 to Scheper et al and U.S. Pat. App. No. 2005/0067300 to Tremblay. The device may contain an electrochemical cell to generate dilute hypohalous acid and a mechanism to evaporate the hypohalous acid solution into the air. The electrochemical cells and/or electrolytic devices are those cells and/or devices that are self-powered and self-contained and which draw their electrical power from the unattached electrolytic device itself and/or alternatively from a building's electrical power supply to produce electrolyzed water. The device can be plugged in or can contain power to supply for the electrochemical cell, the power for any pumping means, the power for any propulsion means, the power for any indication or control means, and the like. The devices can comprise a housing that can be sealed or can be sealable to prevent electrolytic solution from entering the housing, except as intended. The body can have an inlet port, through which electrolytic solution can pass through to the electrochemical cell, contained therein.

### Full Room Treatment and Personal Devices

An aerosol device can be placed in the center of a room, then the aerosol device is activated and in a few minutes the entire contents are expelled and the air and surfaces of the room to kill germs and remove allergens. The aerosol device can be safe to use around kids, pets, and food. As an alternative to the aerosol device, a canister containing the active with a fan or a canister with a heat generating mechanism to deliver the active. This technology can also be used to deliver dilute hypohalous acid to a person. Suitable personal devices to deliver actives for respiratory treatment are disclosed in PCT App. No. WO0162264 to Zawadzki et al., which describes suitable dispensers including self-milling dry powder dispensers for actives as described in U.S. Pat. App. No. 2005/0233900. These personal device can be use to deliver dilute hypohalous acid in a liquid nebulisers or dry powders containing hypohalous acid.

The device may be a self-generating plug-in or portable device, for example as described in U.S. Pat. App. No. 2003/0213704 to Scheper et al. and U.S. Pat. App. No. 2005/0067300 to Tremblay. The device may contain an electrochemical cell to generate dilute hypohalous acid. The electrochemical cells and/or electrolytic devices are those cells and/or devices that are self-powered and self-contained and which draw their electrical power from the unattached electrolytic device itself and/or alternatively from a building's electrical power supply to produce electrolyzed water. The device can be plugged in or can contain power to supply for the electrochemical cell, the power for any pumping means, the power for any propulsion means, the power for any indication or control means, and the like. The devices can comprise a housing that can be sealed or can be sealable to prevent electrolytic solution from entering the housing, except as intended. The body can have an inlet port, through which electrolytic solution can pass through to the electrochemical cell, contained therein.

In-situ generation of hypochlorous acid by electrolysis of slowly dissolving salt solution or brine may be a suitable source of hypochlorous acid when it is desired to emit hypochlorous acid vapor for a long period of time. The salt could be added using a stepping motor or screw type device, or the brine solution could be saturated and in equilibrium with excess salt to prolong the generation of hypochlorous acid. The salt could also be replenished in the electrolysis cell via osmosis using a membrane to separate an electrolysis cell with a more dilute salt concentration than in the larger reservoir. The electrolysis can be done using batteries or household current or rectified household current.

Another aspect of the invention is controlling the rate at which the emitter is exhausted so the article emits hypochlorous acid for a specific period of time. In some cases, the article will be designed to emit a high rate of flux to achieve a rapid reduction of microorganisms. This is achieved using a high concentration of hypochlorous acid (which may be formed in-situ) at a pH where a large percentage of the hypochlorite is in the form of hypochlorous acid. This could be used in a doctor's office as an overnight environmental surface sanitizer or disinfectant, elsewhere it would be acceptable to use all the hypochlorous acid in one use period. It may also include a fan or some other mechanical means to disperse the vapor. At the other extreme, a product could be designed to slowly emit hypochlorous acid over a long time to control microorganisms for a long period of time. Such articles could be useful to preserve items such as food or clothing during storage. In another aspect, the article is designed to achieve both initial fast and slowly continuous levels.

### Santizing Tablet

A tablet can dissolve in water to deliver low levels of hypohalous acid at neutral to acidic pH. The tablet may effervesce. The tablet can be used after the kids take a bath by tossing the tablet in a full tub before draining and the tub and bath toys will be sanitized. The tablet can also be used to sanitize the kitchen sink and cutting board, used in a humidifier, washing machine, and dishwasher. The tablet is safe to use around kids, pets, and food.

### Spaces for Treatment

The present invention relates an apparatus or device and method for treatment of air, surfaces, and spaces. The apparatus and method for treatment can be suitable for use in various confined spaces, including, but not limited to, refrigerators, closets, clothes dressers, and the like. When the device is used for active treatment, it is possible to effectively use the device in even larger spaces, such as in a room, or closet. The apparatus and method of the present invention are, however, by no means limited to such uses. For example, it also possible for the device, or a portion thereof, to be used on its own for treating relatively small spaces like the inside of an automobile. The apparatus may also be provided with one or more components that can be used independently to treat the air, surfaces, spaces in other locations.

Confined spaces often have complex structures so that normal air convection does not reach every corner of the confined space. Such complex structures for example include separate compartments such as drawers or hollow elements inside the confined space. In accordance with one aspect of the method of the present invention, it is possible to also treat those portions of the confined space which are not sufficiently accessible to normal air convection. A confined space for which one aspect of the method of the present invention is particularly suitable comprises a compartment (e.g., the vegetable drawer in a refrigerator) which is within a confined space (the refrigerator) but which is separated from the remainder of the confined space (the interior of the refrigerator). With the method of the present invention it is therefore possible to treat all compartments in a confined space such as a refrigerator (which has enclosed compartments for vegetables, meats, etc.), a closet (which has shoe storage closets, clothes storage containers, etc.), or the like.

When used for treatment, the apparatus can provide several benefits, especially in confined spaces such as refrigerators, including, but not limited to: removing malodor from confined spaces; removing ethylene from confined spaces; maintaining the fresh odor of confined spaces; reducing the transfer of airborne bacteria in confined spaces; maintaining the freshness of food items; improving the quality of food items; maintaining the fresh taste of food items; preventing the transfer of odors between two food items; extending the useful life of food items; keeping food items fresh over a longer period of time; reducing spoilage of food items; reducing the incidence of freezer burn of food items in a freezer compartment; maintaining the fresh taste and/or odor of ice cubes (preferably ice cubes made by an automatic ice maker); increasing the cooling. Water washed smectite clays are often preferred because they are controlled for purity, bacteria, whiteness, heavy metals and performance efficiency of a refrigerator; preventing or reducing the formation of ice crystals on ice cream in an opened or partially-sealed box stored in a freezer compartment; and combinations thereof. The present invention further relates to the use of the apparatus to achieve such benefits (i.e. technical effects).

### Optional Ingredients

The compositions may also include minor amounts, generally not more than at total of 1% wt., desirably less than 0.1% wt. of one or more optional constituents including ones which may improve the. Suitable antibacterial metal salts include salts of metals in groups 3b-7b,8 and 3a-5a. Specifically are the salts of aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof. Suitable metallic antimicrobials include silver compounds as described in U.S. Pat. No. 6,180,584 to Sawan.

Suitable phenolic antimicrobials include o-penyl-phenol, o-benzyl(p-chlorophenol), 4-tertamylphenol and mixtures thereof.

Suitable essential oil antimicrobials include those essential oils which exhibit anti-microbial activity. By “actives of essential oils”, it is meant herein any ingredient of essential oils that exhibit anti-microbial activity. It is speculated that said anti-microbial essential oils and actives thereof act as proteins denaturing agents. Such anti-microbial essential oils include, but are not limited to, those obtained from thyme, lemongrass, citrus, lemons, orange, anise, clove, aniseed, pine, cinnamon, geranium, roses, mint, lavender, citronella, eucalyptus, peppermint, camphor, ajowan, sandalwood, rosmarin, vervain, fleagrass, lemongrass, ratanhiae, cedar and mixtures thereof. Suitable anti-microbial essential oils to be used herein are thyme oil, clove oil, cinnamon oil, geranium oil, eucalyptus oil, peppermint oil, citronella oil, ajowan oil, mint oil or mixtures thereof. Actives of essential oils to be used herein include, but are not limited to, thymol (present for example in thyme, ajowan), eugenol (present for example in cinnamon and clove), menthol (present for example in mint), geraniol (present for example in geranium and rose, citronella), verbenone (present for example in vervain), eucalyptol and pinocarvone (present in eucalyptus), cedrol (present for example in cedar), anethol (present for example in anise), carvacrol, hinokitiol, berberine, ferulic acid, cinnamic acid, methyl salicylic acid, methyl salycilate, terpineol, limonene and mixtures thereof. Suitable actives of essential oils to be used herein are thymol, eugenol, verbenone, eucalyptol, terpineol, cinnamic acid, methyl salicylic acid, limonene, geraniol or mixtures thereof.

Suitable oxidant antimicrobials include hydrogen peroxide and other peroxides, sources of hydrogen peroxide and other peroxides, generators of hydroxyl radical, peracid bleaches and peracid bleach precursors, as described in U.S. Pat. No. 6,548,467 to Baker et al. and U.S. Pat. No. 6,627,590 to Sherry et al.

Suitable acid antimicrobials include: citric acid, cresylic acid, dodecylbenzene sulfonic acid, phosphoric acid, salicylic acid, sorbic acid, sulfamic acid, acetic acid, benzoic acid, boric acid, capric acid, caproic acid, cyanuric acid, dihydroacetic acid, dimethylsulfamic acid, propionic acid, polyacrylic acid, 2-ethyl-hexanoic acid, formic acid, fumaric acid, 1-glutamic acid, isopropyl sulfamic acid, naphthenic acid, oxalic acid, phosphorus acid, valeric acid, benzene sulfonic acid, xylene sulfonic acid, as well as any acid listed as a registered pesticide active ingredient with the United States Environmental Protection Agency. Further useful acids include: sulfonic acids, maleic acid, acetic acid, adipic acid, lactic acid, butyric acid, gluconic acid, malic acid, tartaric acid, as well as glycolic acid. Desirably glycolic acid and citric acid are used as they are effective and in plentiful supply.

Antimicrobial agents are present, suitably at levels below about 0.5%, or below about 0.4%, or below 0.1%.

### Other Product Components

Other suitable components in any suitable amount may be used. Suitable ingredients include, but are not limited to: aesthetic appeal of the compositions, viz., perfumes and colorants. These optional ingredients may be present in larger amounts if they are kept physically separated from the hypohalous acid composition during long-term storage. Such optional constituents should not undesirably affect the shelf stability or rheology of the compositions. By way of non-limiting example such further constituents include one or more coloring agents, fragrances and fragrance solubilizers, viscosity modifying anti-filming agents, other surfactants, pH adjusting agents and pH buffers including organic and inorganic salts, optical brighteners, opacifying agents, hydrotropes, antifoaming agents, antideposition agents, anti-spotting agents, preservatives, and anti-beads, binders, bleach activators, bleach catalysts, bleach stabilizing systems, bleaching agents, brighteners, buffering agents, builders, carriers, chelants, clay, color speckles, control release agents, corrosion agents. The use and selection of these optional constituents is well known to those of ordinary skill in the art: inhibitors, dishcare agents, disinfectant, dispersant agents, dispersant polymers, draining promoting agents, drying agents, dyes, dye transfer inhibiting agents, enzymes, enzyme stabilizing systems, fillers, free radical inhibitors, fungicides, germicides, hydrotropes, opacifiers, perfumes, pH adjusting agents, pigments, processing aids, silicates, soil release agents, suds suppressors, surfactants, stabilizers, thickeners, zeolite, and mixtures thereof.

Where the composition is used to treat mold or other microbiological contaminants, the addition of other agents that have short-term or long-term effectiveness against these contaminants may be included. For example, octaborate is known to be effective against the reoccurrence of mold and mildew.

### Delivery

The compositions of the invention can be delivered via bottle, spray, aerosol, or a directed flow such as the bleach pen as in U.S. Pat. No. 6,905,276. The compositions of the invention can be delivery via devices described in U.S. Pat. App. No. 2005/0221113 and U.S. Pat. App. No. 2005/0232848. The compositions of the invention can be delivered as part of a multi-compartment delivery system, for example as described in U.S. Pat. No. 5,954,213, U.S. Pat. No. 5,316,159, WO2004/014760, U.S. Pat. No. 6,610,254, and U.S. Pat. No. 6,550,694.

### Efficacy

Dilute sprayable hypochlorite bleach formulations (less than about 0.5% sodium hypochlorite) with a neutral pH are effective sanitizing and disinfecting agents. However, because these formulations do not possess cling properties they tend to runoff vertical surfaces or drip from overhead surfaces like ceilings. This reduces the amount of actives in contact with those surfaces and makes their application somewhat limited. Stain removal efficacy (especially mildew stain removal) of neutral, dilute sprayable hypochlorite bleach formulations (less than about 0.5% sodium hypochlorite) is improved by the addition of small amounts of inorganic thickener such as clay. The inorganic thickener imparts thixotropic properties to the bleach formulation, such that it overcomes the limitations inherent to non-thickened solutions. Because of the increased contact time, the dilute compositions are effective, and the compositions may avoid some of the negatives, such as odor, associated with higher concentrations of actives.

Potential uses for the inventive packaging, compositions, and methods include dishwashing, for example U.S. Pat. Appl. 2003/0216271 to Scheper et al.; hospital environments and medical instruments, for example U.S. Pat. No. 6,632,347 to Buckley et al. and U.S. Pat. No. 6,126,810 to Fricker et al.; wound healing, for example U.S. Pat. Appl. 2003/0185704 to Bernard et al. and U.S. Pat. No. 6,426,066 to Najafi et al.; disinfecting or sterilizing objects such as medical instruments, for example U.S. Pat. No. 6,623,695 to Malchesky et al.; disinfecting and deodorizing the air, for example U.S. Pat. Appl. 2002/0179884 to Hoshino et al.; for water purification, for example U.S. Pat. No. 6,296,744 to Djeiranishvili et al.; removal of mold and mildew, for example U.S. Pat. No. 5,281,280 to Lisowski et al.

Co-pending application Ser. No. 10/838,571, filed Apr. 23, 2004 discloses factors in the chemical composition that affect the stability of dilute hypohalous acid and hypohalous acid salt compositions, and is incorporated by reference. The stability of these compositions is also affected by packaging and manufacturing materials.

### EXAMPLES

### Hypochlorite Dilution Examples

Table I shows that diluted hypochlorite solutions have good stability at near neutral pH, especially when diluted with water relatively free from metal ions, salts, and total organic carbon (TOC) (Table II). The initial concentration of the concentrated sodium hypochlorite was 6.24% sodium hypochlorite and the stability samples were stored in 174 oz. Clorox® bleach bottles.

TABLE I

| | | Loss at | Loss at | |
|---|---|---|---|---|
| | Initial | 120° F. | 120° F. | |
| | available | after | after 19.7 | Loss at 120° F. |
| | chlorine | 9.9 days | days | after 30.7 days |
| Conc. | 84.2 (pH 7.53) | 17.1% | 23.7% | 33.4% (pH 7.84) |
| Hypochlorite | | | | |
| and tap water | | | | |
| Conc. | 83.0 (pH 7.53) | 19.3% | 22.8% | 26.0% (pH 8.11) |
| Hypochlorite | | | | |
| and Soft water^{a} | | | | |
| Conc. | 82.3 (pH 7.53) | 11.7% | 17.7% | 23.1% (pH 7.10) |
| Hypochlorite | | | | |
| and DDI water^{b} | | | | |
| Conc. | 83.3 (pH 7.53) | 10.9% | 16.9% | 22.0% (pH 7.18) |
| Hypochlorite | | | | |
| and DI water^{c} | | | | |
| Conc. | 83.0 (pH 7.53) | 11.0% | 15.9% | 19.4% (pH 7.52) |
| Hypochlorite | | | | |
| and RO water^{d} | | | | |
| Conc. | 85.0 (pH 7.53) | 11.8% | 17.3% | 22.1% (pH 7.20) |
| Hypochlorite | | | | |
| and RO/DI | | | | |
| water^{e} | | | | |

Soft water from a sodium cation exchange process.
DDI is deionized and then distilled water.
DI is from a hydrogen cation exchange process.
RO is from a reverse osmosis process.
RO/DI is from reverse osmosis and then a hydrogen cation exchange process.

TABLE II

| Water source | TOC, ppm | Cu, ppb |
|---|---|---|
| Tap Water | 0.702 | 129 |
| Soft Water | 3.030 | <70 |
| DDI Water | Not measured | Not measured |
| DI Water | 0.065 | <70 |
| RO Water | 0.052 | <70 |
| RO/DI Water | 0.059 | <70 |

Table III shows that diluted hypochlorite solutions have good stability at near neutral pH, especially when diluted with water relatively free from metal ions and salts. The solutions also have good stability in the presence of chelants, such as pyrophosphate and orthophosphate. The initial concentration of the concentrated sodium hypochlorite was 6.448% sodium hypochlorite.

TABLE III

| | Initial av. | | |
|---|---|---|---|
| | Chlorine | Loss at 70° F. | Loss at 120° F. |
| | in ppm (pH) | after 27 days | after 27 days |
| Conc. Hypochlorite and | 79 (pH 7.6) | 7% | 52% |
| tap water | | | |
| Conc. Hypochlorite and | 77 (pH 7.5) | 0% | 22% |
| distilled deionized water | | | |
| Conc. Hypochlorite, | 81 (pH 7.6) | 6% | 25% |
| 23 ppm Orthophosphate, | | | |
| distilled deionized water | | | |
| Conc. Hypochlorite, | 80 (pH 7.6) | 4% | 29% |
| 11.5 ppm Pyrophosphate, | | | |
| distilled deionized water | | | |

Table IV shows compositions of the invention with impurity concentrations. Low concentrations of these impurities can enhance the stability of the compositions. In some cases, the initial concentrations of the impurities may be higher and the impurities may be made less reactive or inert over time. In these cases, the compositions may have increased stability upon aging.

TABLE IV

| | Diluted | | | |
|---|---|---|---|---|
| | hypochlorite | | Diluted hypochlorite | |
| Available chlorine, ppm | 200 | ppm | 40 | ppm |
| pH | 7.9 | | 5.1 | |
| Copper | <100 | ppb | <80 | ppb |
| Nickel | <10 | ppb | <8 | ppb |
| Cobalt | <30 | ppb | <20 | ppb |
| Total organic carbon | <500 | ppb | <200 | ppb |

Table V shows that dust mite allergens are effectively denatured with diluted hypochlorite solutions down to 5 ppm available chlorine. The pH obtained for diluted hypochlorite solution at 4 ppm was 6.51. The compositions are also effective against allergens within 30 seconds.

Product efficacy screening was performed by using a modified antibody capture ELISA (where a recombinant antigen is coated onto polystyrene, the product is added directly to predetermined wells and incubated for a selected period of time, the results of the product treated wells are compared against those of untreated wells, the concentration is calculated against a standard curve). This method differs from the antigen capture ELISA in that product interference which affected antibody-antigen complex is not considered because the product is added directly to the antigen/allergen, the wells are washed of excess product and the labeled antibody is incubated onto the remains of the antigen. Protein fragmentation was revealed by SDS-PAGE method and loss of Allergenic activity (antibody binding to antigen) was observed in Western blot (immunoblot).

TABLE V

| | Available chlorine, | Dust mite allergen, % |
|---|---|---|
| | ppm | reduction |
| Diluted hypochlorite | 0.77 | 75 |
| | 4.0 | 98 |
| | 7.8 | 99 |
| | 19.4 | 100 |
| | 38.4 | 100 |
| | 57.7 | 100 |
| | 77 | 100 |
| | | (30 sec) |

Table VI shows that diluted hypochlorite is effective at sanitizing and disinfecting as measured by efficacy against Staphylococcus aureus. Tests were conducted using the AOAC Germicidal Spray Products test method (AOAC 961.02, 15th edition, SOP No. 001-057-06). An approximate 48-hour suspension of Staphylococcus aureus grown up in AOAC Synthetic Broth was used for testing. The culture concentration was adjusted to yield a target of 4×10⁴ per slide once dried. For the runs to be conducted with organic soil load, a separate bacteria suspension was prepared with fetal bovine serum where the serum load was 5%. A volume of 0.01 ml was inoculated per glass slide. A sterile bent needle was used to spread the inoculum to within ⅛″ from the edge. For each inoculation run, the slides were dried in the 35° C. incubator until completely dry. Prior to testing, bottle caps were replaced with trigger sprayers. The triggers were primed and testing was started by spraying the contaminated surfaces from 6-8 cm distance for 2-3 seconds. The surface was completely wet by about 3-4 full pumps. The amount of product that was dispensed per trigger ranged from 2.24 g to 2.90 g. For the samples that were pipeted onto the contaminated surfaces, the dispensing volume was between 2.5 ml per slide (with filter paper) and 5 ml per slide (without filter paper).

TABLE VI

| | | | Sample with |
|---|---|---|---|
| | Available chlorine in | | residual |
| | ppm | pH | bacteria |
| Diluted | 707.6 | 9.70 | 0/60 |
| hypochlorite | | | |
| Diluted | 63.4 | 7.36 | 0/60 |
| hypochlorite | | | |
| After storage | | | |
| 120 F. for 1 month | | | |

Table VII shows that the compositions are effective at killing a variety of viruses and spores.

TABLE VII

| | Diluted hypochlorite |
|---|---|
| Polio I Virus | Effective |
| Influenza A Virus | Effective |

The compositions are effective at controlling mold growth. Diluted hypochlorite tested against penicillium mold in a petri dish gave growth inhibition.

The dilute hypochlorite compositions are effective at controlling odors. Dilute hypochlorite can control odors by both killing the odor-causing bacterial as well as oxidizing the odor molecules themselves, breaking them down into smaller, odorless components. An initial test was done using garlic juice in small plastic containers. A drop of garlic juice was placed in each of two plastic containers at room temperature and allowed to equilibrate for 10 minutes. The containers are then opened and one is sprayed with dilute hypochlorite and one with plain water. The containers were then closed and again allowed to equilibrate for 10 minutes. Then a corner of the container is opened to smell the contents. The containers sprayed with dilute hypochlorite had less garlic odor than the one sprayed with water.

The compositions of the invention can give minimal fabric damage compared to other hypochlorite compositions. Cotton, rayon, and wool were sprayed with dilute hypochlorite until damp and allowed to dry between sprayings. Test was repeated for upwards of 20+ sprays. No visible damage was observed. Swatches of bleach sensitive blue-dyed cotton (Intralite Turquoise GL) were soaked in dilute hypochlorite solutions. Swatches showed no discoloration for several hours. Some bleaching was observed when soaked for longer times and was easily observable after 24 hours.

The composition of the invention was found to kill Aspergillus fumigatus Conidia spores in solution and to inactivate Aspergillus fumigatus Conidia antigen in solution. The composition was also tested on hard surfaces. The composition of the invention was found to reduce mold growth on drywall 6 logs compared to water (none). The composition of the invention was found to reduce mold growth on plywood 6 logs compared to water (none). The composition of the invention was found to reduce mold growth on oriented strand board more than 6 logs compared to water (none). The compositions of the invention were tested for in vivo allergic response in humans, wherein said test method comprises the subcutaneous injection of allergens treated with the composition. The residue after treatment on oriented strand board was evaluated by prick skin testing on test subjects who had a history of positive skin prick to Aspergillus fumigatus.

Results from the in vivo testing suggest that the inventive compositions will reduce or prevent respiratory ailments caused by allergens and reduce or prevent allergies.

### Hypochlorite Multilayer Bottle Examples

During the course of evaluating various oxidants and antimicrobials for their allergen deactivating ability, we have found that a very dilute solution (on the order of 40-80 ppm) of primarily hypochlorous acid can effectively deactivate allergens. Presumably these low levels of oxidant are still able to break up the allergen proteins, rendering them biologically inert.

While effective, the low concentration and nearly neutral pH (6.9) of hypochlorous virtually eliminates damage to surfaces. There is no sticky residue that can affect the feel of fabrics and there may be minimal dye damage. The solution may be aerosolized to treat air directly, or applied to surfaces. Aerosols are known to have a low collision rate between denaturant and allergen particles. As a result, the denaturant must be used in high concentrations to be effective. Using this approach with conventional denaturants, which may be irritating or fragranced at high levels, can cause health problems.

Co-pending application Ser. No. 10/828,571, filed Apr. 20, 2004 discloses factors in the chemical composition that affect the stability of dilute hypohalous acid and hypohalous acid salt compositions, and is incorporated by reference. The stability of these compositions is also affected by packaging and manufacturing materials.

Concentrated hypochlorite bleach is commonly stored in opaque HDPE containers and is not typically compatible with PET containers. Dilute hypochlorite compositions are stable PET containers. The stability of dilute hypochlorite compositions in containers is affected by plastic additives, for example Kemamide® slip agent in polyethylene. The stability of dilute hypochlorite compostions in containers is affected by copolymer blends, for example, acetal copolymers such as Celcon® M90.

It might be expected that opaque monolayer HDPE bottles might protect dilute hypochlorite compostions from sunlight exposure. However, in these HDPE bottles, UV exposure accelerates the degradation of dilute hypochlorite compositions despite a minimal transmission of UV and visible light thru the opaque HDPE bottles. We have found that multilayer bottles with additives in the intermediate or outside layer provide improved stability over single layer bottles. Table VIII shows stability results of 200 ml HDPE bottles, both multilayer and monolayer, which degraded under UV exposure in the window. The control bottle was kept in the dark.

TABLE VIII

| | 1 week | 2 weeks | 3 weeks | 4 weeks |
|---|---|---|---|---|
| Control (Trilayer bottle with virgin | 100% | 99% | 99% | 96% |
| resin interior layer kept in dark) | | | | |
| Monolayer bottle with colorant - | 90% | 76% | 68% | 63% |
| exposed to light | | | | |
| Trilayer bottle with virgin resin | 97% | 95% | 92% | 87% |
| interior layer - exposed to light | | | | |

Trilayer bottles where the outer layer or intermediate layer has an additive from the group of opacifiers, colorants, and UV inhibitors and where the inner layer has a substantially lower concentration of one of these additives compared to the outer layer or intermediate layer have substantially greater stability compared to bottles where these additives are in the layer that directly contacts the dilute hypochlorite solution. An example of such a trilayer bottle and a bilayer bottle is given in Table IX.

TABLE IX

| | Bottle type | |
|---|---|---|
| | | Extrusion, blow-molded, |
| | Extrusion, blow-molded, HDPE | HDPE |
| Wall | 30 mils (15% inner layer, 70% | 30 mils (15% inner layer, |
| thickness | middle layer, 15% outer layer) | 85% outer layer) |
| Outer layer | 3% colorant - pigment including | 3% colorant - pigment |
| | titanium dioxide | including titanium |
| | | dioxide |
| Middle layer | 1% colorant, 35% PCR (post- | None |
| | consumer resin) | |
| Inner layer | 0% colorant, virgin resin | 0% colorant, virgin resin |

Dilute hypochlorite compositions are UV and light sensitive. UV absorbers that inhibit up to 390 nm can be required for long-term stability in normal store shelf lighting. Light protection up to the 550 nm can be required for direct sunlight exposure through a window. We have found that colorants in plastic bottles affect bleach stability. Therefore, in order to achieve stability from sunlight exposure, a solid color printed on plastic film such as a shrink sleeve or a tinted plastic film such as a shrink sleeve can be used to protect from UV radiation, yet avoid stability problems when the colorant is in the plastic container. One solution to packaging stability of dilute hypochlorite compositons is to use removable printed shrink sleeve that communicates at shelf and then is removed to reveal an aesthetic bottle underneath when peeled away.

We have found that lowering the pH of the formula improves UV stability. The pH of the dilute hypochlorite composition can be lowered from pH 7.5 to pH 5.5 to provide additional stability against UV radiation. The bottles were tested under accelerated testing for 24 hours in the FadeOmeter® at 130° F. with the results in Table X.

TABLE X

| | pH 7.5 | pH 5.5 |
|---|---|---|
| 121 ppm sodium | 56% | 70% |
| hypochlorite in PET bottle | | |
| with UV inhibitor | | |

### Dry Hypochlorite Examples

Co-pending application Ser. No. 10/828,571, filed Apr. 20, 2004 discloses factors in the chemical composition that affect the stability of dilute hypohalous acid and hypohalous acid salt compositions, and is incorporated by reference. The stability of these compositions is also affected by packaging and manufacturing materials.

Aerosil R812S® from and Cab-O-Sil TS 720° from have adequate substitution of surface silanol groups to convert solutions with 0-7% NaOCl with a pH below about 11.8 to powders, as seen in Tables XI and XII. Aerosil R812® has less carbon than Aerosil R812S° which indicates Aerosil R812® has more unblocked surface silanol groups. The results with Aerosil R812® are shown in Table XIII.

TABLE XI

| | Hypochlorite Solution | | | | | |
|---|---|---|---|---|---|---|
| | | | | % NaOCl | Cab-O-Sil TS 720 | |
| Trial | % NaOCl | pH | g used | in powder | g used | % in powder |
| 1 | 0.0100 | 5.14 | 38.99 | 0.0095 | 2.00 | 4.88 |
| 2 | 0.0205 | 6.81 | 41.67 | 0.0198 | 1.54 | 3.56 |
| 3 | 0.0205 | 7.00 | 202.49 | 0.0196 | 9.13 | 4.31 |
| 4 | 0.0202 | 7.54 | 40.77 | 0.0193 | 2.08 | 4.85 |
| 5 | 0.0204 | 9.45 | 42.05 | 0.0194 | 2.07 | 4.69 |
| 6 | 1.60 | 9.17 | 40.42 | 1.53 | 2.04 | 4.80 |
| 7 | 6.33 | 10.38 | 46.00 | 6.04 | 2.19 | 4.54 |
| 8 | 6.33 | 11.06 | 47.58 | 6.05 | 2.17 | 4.36 |
| 9 | 6.33 | 11.41 | 44.81 | 6.05 | 2.05 | 4.37 |
| 10 | 6.33 | 11.87 | 40.20 | 6.02 | 2.04 | 4.83 |

TABLE XII

| | Hypochlorite Solution | | | | Aerosil R812S | | |
|---|---|---|---|---|---|---|---|
| | % | | | % NaOCl | | % in | |
| Trial | NaOCl | pH | g used | in powder | g used | powder | Powder |
| 1 | 0.0100 | 5.14 | 40.00 | 0.0096 | 1.81 | 4.33 | Yes |
| 2 | 0.0205 | 6.81 | 52.02 | 0.0198 | 1.77 | 3.29 | Yes |
| 3 | 0.0205 | 7.00 | 227.62 | 0.0196 | 10.14 | 4.26 | Yes |
| 4 | 0.0205 | 6.81 | 496.82 | 0.0197 | 20.63 | 3.99 | Yes |
| 5 | 0.0981 | 5.21 | 40.66 | 0.0939 | 1.80 | 4.24 | Yes |
| 6 | 0.991 | 11.43 | 40.44 | 0.945 | 1.95 | 4.59 | Yes |
| 7 | 6.33 | 11.37 | 40.12 | 6.00 | 2.20 | 5.20 | Yes |
| 8 | 6.33 | 11.55 | 40.51 | 6.03 | 2.01 | 4.73 | Yes |

TABLE XIII

| | Hypochlorite Solution | | | | Aerosil R812 | | |
|---|---|---|---|---|---|---|---|
| | % | | | % NaOCl | | % in | |
| Trial | NaOCl | pH | g used | in powder | g used | powder | Powder |
| 1 | 0.412 | 3.39 | 42.79 | 0.394 | 2.04 | 4.86 | Yes |
| 2 | 0.264 | 3.39 | 41.99 | 0.251 | 2.15 | 4.86 | Yes |
| 3 | 0.694 | 4.60 | 40.05 | 0.661 | 1.97 | 4.69 | Yes |
| 4 | 0.303 | 4.80 | 42.40 | 0.289 | 2.05 | 4.86 | Yes |
| 5 | 0.0100 | 5.14 | 39.41 | 0.0095 | 1.99 | 4.81 | Yes |
| 6 | 0.0981 | 5.21 | 40.49 | 0.0934 | 2.01 | 4.73 | Yes |
| 7 | 0.345 | 5.51 | 42.71 | 0.329 | 2.09 | 4.86 | Yes |
| 8 | 0.0202 | 5.80 | 125.37 | 0.0192 | 6.50 | 4.93 | Yes |
| 9 | 0.206 | 5.81 | 41.46 | 0.197 | 1.98 | 4.86 | Yes |
| 10 | 0.463 | 5.84 | 40.09 | 0.442 | 1.98 | 4.71 | Yes |
| 11 | 0.620 | 5.87 | 40.36 | 0.591 | 1.99 | 4.70 | Yes |
| 12 | 0.401 | 6.06 | 43.10 | 0.382 | 2.07 | 4.86 | Yes |
| 13 | 0.311 | 6.08 | 42.69 | 0.297 | 2.03 | 4.86 | Yes |
| 14 | 0.223 | 6.52 | 41.72 | 0.213 | 2.04 | 4.86 | Yes |
| 15 | 0.0202 | 7.54 | 125.08 | 0.0192 | 6.59 | 5.00 | Yes |
| 16 | 0.148 | 7.62 | 40.02 | 0.141 | 2.00 | 4.76 | Yes |
| 17 | 0.0204 | 9.45 | 125.06 | 0.0194 | 6.53 | 4.96 | Yes |

Cab-O-Sil TS 530® and HDK H2000® from are similar to Aerosil R812® and Aerosil R812S®, and powders of hypochlorite solutions have been made from these treated fumed silicas as seen in Table XIV

TABLE XIV

| | Trial | | | | |
|---|---|---|---|---|---|
| | 1 | 2 | 3 | 4 | 5 |
| % NaOCl in solution | 0.0201 | 0.0201 | 6.20 | 0.0201 | 0.0201 |
| % Boric acid in | | | | 0.0995 | 0.0995 |
| solution | | | | | |
| pH of solution | 5.80 | 5.80 | 11.31 | 5.76 | 5.76 |
| Solution used, g | 40.19 | 40.75 | 40.31 | 125.29 | 125.30 |
| Aerosil R812S, g | | | 1.01 | 5.99 | |
| Aerosil R812, g | | | | | 6.65 |
| HDK H2000, g | | 2.28 | | | |
| Cab-O-Sil TS-530, g | 2.02 | | | | |
| Cab-O-Sil TS-720, g | | | 0.99 | | |
| % treated silica in | 4.79 | 5.30 | 4.73 | 4.56 | 5.04 |
| powder | | | | | |
| % NaOCl in powder | 0.0191 | 0.0190 | 5.91 | 0.0190 | 0.0189 |

Mixed using Omni GLH homogenizer with 20 mm disperser polypropylene jars
Aerosil R812S ® from Degussa AG
Cab-O-Sil TS 720 ® from Cabot Corp.
HDK H2000 ® from Wacker Chemical Corp.

The amount of treated silica required to convert salt solutions to powders is between 3 and 6% by weight of the final composition. Amounts greater than 6% can be used, but the excess does not participate in particle formation. The optimum amount depends on the pH and ionic strength of the salt solution and on the type and extent of treatment on the fumed silica. It may also depend on the method of production. With Aerosil R812S® and Cab-O-Sil TS 720® a suitable amount is 3.5-5.5 weight percent of the finished powder; or 4-5%. With less amount of treated silicas, the powder can be difficult to form, and with more the excess of treated silica may be present as a fine dust.

Powders have also been made using salts other than sodium hypochlorite (lithium chloride, magnesium sulfate, and potassium nitrate) and with mixtures of salts as shown in Table XV. It must be remembered that the sodium hypochlorite used in this work contains an equimolar amount of sodium chloride and a small amount of sodium carbonate.

TABLE XV
Powdered Salt Solutions Made With Aerosil R812S

| Salt Solution | | | Aerosil R812S | % in |
|---|---|---|---|---|
| Salt | mol/kg | g used | g used | powder |
| LiCl | 0.304 | 40.90 | 1.96 | 4.57 |
| K2SO4 | 0.371 | 39.74 | 1.95 | 4.70 |
| KNO3 | 0.482 | 39.83 | 1.95 | 4.69 |
| MgSO4•7H2O | 0.495 | 40.72 | 1.94 | 4.59 |

Mixed using Omni GLH homogenizer with 20 mm disperser in 4 oz polypropylene jar

The process of converting aqueous salt solutions to powders using treated fumed silica requires shear to break apart the silica agglomerates into their aggregates and to create 1-20 μm droplets of aqueous composition. The treated fumed silica aggregates spontaneously and coats these small water droplets to form the free-flowing powder. Particles as large as 30 μm are found, but most are often smaller than 10 μm.

Coated particles of salt solutions are typically formed within 10-200 seconds at 10,000-30,000 using a rotor-stator mixing head. Suitable is a laboratory homogenizer, either a Tekmar Tissuemiser with a 18-N disperser (generator), or an Omni GLH with a 20 mm disperser made of titanium. Rotor stator devices with one or more stages are also available for continuous production in which the salt solution and the treated fumed silica are feed directly into the mixing chamber. Coated powders can also be made using a high speed mixer with various styles of mixing blades. A solution of 0.0085% NaOCl at pH 7.5 was coated with 4.5% of Aerosil R812S® using an Osterizer 10-speed blender on the highest speed. Powders were also made by mixing a solution of 0.02% NaOCl at pH 6.81 with 4.17% Aerosil R812S® at high speed (7500 rpm) using a T-Line Model 101 Mixer with a 4-blade pitched turbine impeller in a straight sided container and by mixing a solution of 0.01% NaOCl at pH 5.1 with 4.16% Aerosil R812S® at high speed (7500 rpm) using a T-Line Model 103 Mixer with a 3-bladed hydrofoil impeller in a straight sided container. Other methods capable of breaking apart the silica agglomerates and forming water droplets smaller than about 20 μm are also suitable. These would include colloid mills, cavitation from ultra sonic generators and high shear fluid processors such as those made by Microfluidics. High shear fluid processors force liquids and powders through specially designed chambers at high pressure to form small particles using high shear and collision impact.

A nonwoven wipe with powdered hypochlorite was made as follows. A powder was made by mixing 50.7 g of a solution with 0.102% NaOCl at pH 5.15 with 50.93 g of deionized water and 4.88 g of Aerosil R812S® in a 250 mL polypropylene beaker. 1.38 g was spread over the surface of a 5″ square of nonwoven polypropylene that weighed 0.66 g (TO-524 PP SMS, 41 g/m² from BBA Nonwovens®). After shaking of the excess, 0.21 g of powder remained on the wipe. When rubbed on a counter, the hypochlorite solution was released to leave a thin layer of liquid.

Powdered hypochlorite was shown to disinfect hard surfaces as follows. A powder was made from 97.7 g of a solution with 0.0085% NaOCl at pH 7.5 and 4.49 g of Aerosil R812S® using a Tekmar Tissuemiser with a 18-N disperser in a 250 mL polypropylene beaker. This was used to kill bacteria on ceramic tile. A culture of Klebsiella species was applied to 2″ diameter circles in the middle of a series of 4″ square black ceramic tiles and allowed to dry. These tiles various treatments with a contact time of four minutes. After four minutes, the center of the tiles were rubbed with a swab that was saturated with soium thiosulfate solution and then touched to the center of an agar plate. The agar plates were sealed and incubated over night at ambient temperature. The next day they were checked for microbial growth. The untreated control had bacterial growth, TNTC. The positive control from a tile that was sprayed with a 2% solution of sodium hypochlorite had no bacterial growth. Bacterial growth, TNTC, was observed when powdered hypochlorite was applied to a tile without rubbing, so no liquid was released from the powder. When the powder was applied to a disposable lab wipe and the treated wipe was used to wipe the tile a few times, liquid was released, and no bacterial growth was observed on the agar plate. The test was repeated with two other types of bacteria, Staphococcus species, and Escherica coli. The powdered bleach was made from 95.46 g of hypochloriote solution and 4.86 g of Aerosil R812S® as before. The results were the same with both types of bacteria. The untreated control had bacterial growth, TNTC, and the positive control which was treated with 2% NaOCl had no growth. Either 0.25 g of powdered hypochlorite was applied directly to the tile and then wiped or 0.25 g of powdered hypochlorite was applied to a lab wipe which was then used to wipe the tile. In both cases there was no growth on the agar plates. An additional test was done in which the tile was rubbed only with a clean lab wipe had bacterial growth, TNTC.

Hypochlorous acid vapors emitted from powdered hypochlorite or from hypochlorite solutions also inhibited mold growth inside sealed Gladware® containers. A 80 mm i.d. mold plate was filed with potato dextrose gel and placed inside a 739 mL Gladware Entree® container, with inside dimensions of 155 mm×155 mm×50 mm deep. A 10 mL glass beaker with the hypochlorite source was also placed inside the container. The lid was placed on top of the container and a swab which had been contaminated with Penicillium species was inserted beneath the lid and shook. The swab was removed and the lid was sealed. The containers were incubated four days at room temperature and visually evaluated for mold growth. The control with no hypochlorite source was completely covered with mold. The container with 2 g of a 0.1% NaOCl solution at pH 5.2 had very little if any mold growth. The containers with 0.5 g of the same hypochlorite solution diluted with 0.5 g of deionized water or with 1 g of a powder made from 50.7 g of the above hypochlorite solution, 50.93 g of deionized water and 4.88 g of Aerosil R812S® had a little mold growth, but much less than the control. These two treatments were nearly identical, which shows the partial pressure of hypochlorous acid in the powder is similar to that of the solution. Thus, increasing the amount of powder or hypochlorite concentration in the powder will completely control the mold as observed in the first treatment. Other treatments had either 0.5 g of the powder described above, or 1 g of a powder made from 95.46 g of a solution with 0.0085% NaOCl at pH 7.5 and 4.86 g of Aerosil R812S®.

Powdered hypochlorite can also be used to pretreat laundry. A powder was made by mixing 60.04 g of a solution with 0.05% NaOCl at pH 5.5 with 2.89 g of Aerosil R812S®. Stained flags were treated by applying ¼ teaspoon (about 0.7 g) to each stain and scrubbing 30 times. After 5 minutes six flags, including untreated flags, were added to a typical top loading washing machine with 69 L of 93° F. and 92.4 g of Liquid Tide® Laundry Detergent. After a normal 12 minute wash the flags were rinsed with 68° F. water and then dried. Stain removal was determined from colorimetric reflectance readings taken before treatment and after drying and converted to % SR(E). The respective % SR(E) for the treated and the control flags for fountain pen ink were 60 and 50, for ball point pen ink were 95 and 35, and for sebum were 73 and 66. Thus, the powdered hypochlorite significantly improved the removal of these stains.

Humidifier Sanitization

In separate experiments, dilute hypochlorite and water were placed in a humidifier in an enclosed 6 by 6 by 6 ft room. Petri dishes containing TSA agar inoculated with S. aureus were placed 30 inches and 60 inches from the humidifier. The humidifier was run for 1.5 hour. A 2 to 5 log reduction was observed on incubated plates placed in the room with dilute hypohalous acid compared to the water control.

Two different humidifiers were used, a Reli-on Ultrasonic Humidifier Model H-0565-0 with nickel transducer and a Fujitronic Ultrasonic Humidifier Model FB-602 with titanium transducer. As shown in Table XVI below, the Relion Humidifier caused a significant drop in the pH of the hypochlorite solution, indicating possible interaction with the nickel transducer and/or the materials that comprised the water container and transducer housing.

TABLE XVI

| | Humidifier | | | |
|---|---|---|---|---|
| | Reli-on | | Fujitronic | |
| | Run Time | | | |
| | Initial | 1.5 Hours | Initial | 1.5 Hours |
| Weight if Solution in | 1000 | 410 | 1000 | 465 |
| humidifer (g) | | | | |
| Hypochlorite concentration | 123 | 82 | 123 | 79 |
| (ppm) | | | | |
| pH | 5.52 | 4.77 | 5.52 | 5.42 |

### Effect of Concentration and pH on Safety

An ultrasonic humidifier was run with bleach diluted to moderate concentration with deionized water and high pH and with low concentration and neutral pH. Black cloth was placed under the humidifier to measure dye damage. The moderate concentration bleach had extensive dye damage, while the low concentration bleach had none, as shown in Table XVII.

TABLE XVII

Humidifier
Kaz Ultrasonic
Humidifier, 5.5 hours

| | | |
|---|---|---|
| Hypochlorite concentration (ppm) | 3759 | 78 |
| pH | 10.7 | 7.35 |
| Dye damage | Yes | No |

### Microbial Control Using Hypochlorous Acid Vapor

Table XVIII represents calculated (estimated using literature equilibrium constants and thus only approximate) chlorine vapor for regular and low salt bleach at constant hypochlorous acid vapor concentration. This table shows that as the pH is raised, it takes a much greater concentration of hypochlorite to give the same hypochlorous acid concentration, but that the ratio of chlorine vapor to hypochlorous acid vapor is also much reduced, especially for low salt hypochlorite. Similar ratios of hypochlorous acid vapor and chlorine vapor are expected from hypochlorite absorbed onto a carrier. Suitable ratios of hypochlorous acid vapor to chlorine vapor may be 250 or greater, or 400 or greater, or 500 or greater, or 550 or greater. Vapor levels of HOCl other about 5 ppm may also be necessary or effective, for example 2 ppm, 10 ppm, 20 ppm, 50 ppm, or 100 ppm. Similar ratios of hypochlorous acid vapor to chlorine vapor may apply.

TABLE XVIII

| | | HOCl vapor | | Cl₂ vapor ppm |
|---|---|---|---|---|
| NaOCl, mg/L | pH | ppm | Cl₂ vapor ppm | Low salt |
| 200 | 5.5 | 5.377 | 0.944 | 0.236 |
| 204 | 6.0 | 5.377 | 0.304 | 0.076 |
| 216 | 6.5 | 5.377 | 0.102 | 0.026 |
| 256 | 7.0 | 5.377 | 0.038 | 0.010 |
| 313 | 7.3 | 5.377 | 0.023 | 0.006 |
| 380 | 7.5 | 5.377 | 0.018 | 0.004 |
| 427 | 7.6 | 5.377 | 0.016 | 0.004 |
| 487 | 7.7 | 5.377 | 0.014 | 0.004 |
| 522 | 7.75 | 5.377 | 0.014 | 0.003 |
| 561 | 7.8 | 5.377 | 0.013 | 0.003 |
| 655 | 7.9 | 5.377 | 0.012 | 0.003 |
| 774 | 8.0 | 5.377 | 0.012 | 0.003 |
| 923 | 8.1 | 5.377 | 0.011 | 0.003 |
| 1110 | 8.2 | 5.377 | 0.010 | 0.003 |
| 1347 | 8.3 | 5.377 | 0.010 | 0.003 |
| 1644 | 8.4 | 5.377 | 0.010 | 0.002 |
| 2018 | 8.5 | 5.377 | 0.010 | 0.002 |
| 2490 | 8.6 | 5.377 | 0.009 | 0.002 |
| 3083 | 8.7 | 5.377 | 0.009 | 0.002 |
| 3830 | 8.8 | 5.377 | 0.009 | 0.002 |
| 4770 | 8.9 | 5.377 | 0.009 | 0.002 |
| 5954 | 9.0 | 5.377 | 0.009 | 0.002 |
| 7445 | 9.1 | 5.377 | 0.009 | 0.002 |
| 9321 | 9.2 | 5.377 | 0.009 | 0.002 |
| 11683 | 9.3 | 5.377 | 0.009 | 0.002 |
| 14657 | 9.4 | 5.377 | 0.009 | 0.002 |
| 18400 | 9.5 | 5.377 | 0.009 | 0.002 |

Experiments have been done to determine the parameters that determine the rate of hypochlorous acid loss from solution. This was done spectrophotometrically and by titration. The mass of hypochlorous acid emitted is governed by pH, concentration, quantity of solution, the height of the solution and the amount of unobstructed surface area.

The presence of hypochlorous acid can be detected by moist starch-iodide indicator paper or by moist available chlorine indicator strips. Electrochemical analyzers that measure available chlorine can be used to measure the concentration of bleach vapors as if they were chlorine. These have been used to demonstrate the presence of hypochlorous in spaces some distance from the emitting solution. The decolorization of dye solutions by the emitted hypochlorous acid has also been followed as a function of time spectrophotometrically.

Hypochlorous acid vapors prevent the growth of mold and kill bacteria that have been deposited onto surfaces, for example in closed containers with volumes between 3 and 132 liters. Bacteria on surfaces behind other objects and not in a direct contact or line of sight, such as behind stuffed toys were killed despite the obstacle of the stuffed toy. Experiments in a 6×6×6 foot chamber demonstrate the inhibition of mold growth. Additional experiments also show that hypochlorous acid vapors can prolong the freshness of fruits and vegetables during refrigerated storage. In a closed container, the vapors may absorb on the surface of the container and provide a residual disinfecting benefit after the hypochlorous acid vapor emitter is removed and the container is reclosed.

In one example, 500 g or 1000 g of 206 ppm hypochlorite bleach at pH 5.52 was put in closed 69 L containers over 12 hours. Glass slides and fabric swatches inoculated with S. aureus were placed 30 cm from the bleach source. The inoculated samples were removed after 12 hours and the there was a 6 log reduction in organisms on both the glass slides and the fabric swatches. In another experiment in a 39 L container, 15 g of 219 ppm hypochlorite was placed in front of a continuous fan and 61 cm away from a polystyrene slide inoculated with S. aureus. After 24 hours, there was a 5 log reduction in organisms. In another experiment, the effectiveness of Gore-Tex® film in reducing water vapor and hypochlorous acid vapor loss was measured. Samples of 200 g of 1061 ppm hypchlorite bleach at pH 6.0 were placed in 14 L containers for 6 hours. One sample covered with Gore-Tex® lost 0.08% water and 1.8% of the hypochlorite. The other uncovered sample lost 0.11% water and 10.6% of the hypochlorite. Samples containing 200 ppm hypochlorite at pH 5.5 were covered with polyester or nylon fabric. These samples showed significantly reduced dye damage on fabric swatches containing bleach sensitive dyes that were placed 16 cm from the hypochlorite samples.

In another experiment, a 75 gm and a 150 gm open container of 6000 ppm hypochlorite at pH 9.0 were tested in separate 132 L enclosures with inoculated glass slides, inoculated fabric, and fabric with bleach sensitive dye placed 32 cm away. After 24 hours, the 75 gm container lost 352 ppm of hypochlorite and the 150 gm container lost 650.7 ppm of hypochlorite. The inoculated glass slide and inoculated fabric in both enclosures showed complete kill. The fabric damage in both enclosures was greatly reduced compared to experiments with pH 5.5 hypochlorite.

Disinfection testing and dye decolorization experiments show that hypochlorous acid vapors released from solutions, solutions absorbed onto fumed silica beads, and solution droplets coated with hydrophobic fumed silica are equally effective, as well as vapors are emitted from gels made using clay thickeners (Laponite®). These gels may be ringing gels that do not flow or spill. Indicator strips show that hypochlorous acid is emitted from solutions heat-sealed into Tyvec® (HDPE) pouches or sealed inside zipper storage bags made of polyethylene. The vapors pass through the polymer film, while the solution remains inside and the outer surface of the pouch remains dry.

Prototypes have been made by putting hypochlorous acid solutions into jars or bottles, heat-sealing such solutions into polyethylene pouches, and enclosing the powder made by mixing the solution with hydrophobic fumed silica into pouches made from nonwoven materials. Delivery devices have also been made by placing a film over a glass jar and holding the film in place with a screw closure ring. Some of the pouches or sachets were equipped with hangers or double sided tape. A prototype was also prepared in which a vial of solution with a wick was attached to a battery operated peizoelectric device that dispenses puffs of mist and vapor. A prototype was prepared by placing an open jar under a battery operated fan in a container that included slits to allow the air to enter from the room and air with hypochlorous acid vapor to be discharged into the room. Other prototypes have been contemplated as described herein. These include a device with a tray of solution under a blower and a device with a reservoir of liquid that is slowly flowed onto an ultrasonic horn to emit fine droplets of solution and vapor. Co-pending application Ser. No. 10/828,571, published as U.S. Pat. App. 2005/0232847 filed Apr. 20, 2004 discloses factors in the chemical composition that affect the stability of dilute hypohalous acid and hypohalous acid salt compositions, and is incorporated by reference. The stability of these compositions is also affected by packaging and manufacturing materials. Co-pending application Ser. No. 11/111,012, published as U.S. Pat. App. 2005/0233900 filed Apr. 21, 2005 discloses dry powdered forms of hypochlorite compositions, and is incorporated by reference.

### Silica Carriers

Table XIX shows silica particles formed by mixing various dilute hypochlorite compositions with hydrophilic silica particles. The hypochlorite compositions (approximately 200 ppm hypochlorite) were stabilized by addition of hydrochloric acid, succinic acid and sodium bicarbonate. The absorbency indicates the weight of aqueous hypochlorite composition that could be absorbed per weight of silica. The silica carrier suitably has an absorbency for 200 ppm hypochlorite solutions of greater than 3, or greater than 5, or about 7 or greater. The stability of the hypochlorite was measured at room temperature (approximately 25° C.) and was captured as percent remaining activity.

TABLE XIX

| Silica | pH | Additive | Absorbency | Stability |
|---|---|---|---|---|
| CE0506 ®¹ | 7 | Succinic | 7 | 37% - 20 days |
| | | acid | | |
| CE0506 ®¹ | 7 | HCl | 7 | 41% - 20 days |
| CE0506 ®¹ | 8.5 | Na | 7 | 11% - 20 days |
| | | Bicarbonate | | |
| Grace Grade 3 | 7 | HCl | 1 | Not determined |
| Grace Grade 59 | 7 | HCl | 2.5 | Not determined |
| CG0602 ®¹ | 5.5 | HCl | 8 | 58% - 7 days |
| CG0602 ®¹ | 7 | HCl | 8 | 53% - 7 days |
| CG0602 ®¹ | 5.5 | Succinic | 7 | 29% - 22 days |
| | | acid | | |
| Aeroperl ® 300/30² | 5.5 | Succinic | 3.4 | 21% - 15 days |
| | | acid | | |
| Aerogel ® TLD302¹ | 5.5 | Succinic | 9.9 | 24% - 22 days |
| | | acid | | |
| Aerogel ® OGD303¹ | 5.5 | Succinic | 9.7 | 34% - 22 days |
| | | acid | | |

Cabot Corp.
Degussa AG.

The type of silica used has a great effect on the amount of bleach absorbed as well as the stability achieved. The CE0506 and the aerogel (OGD303, TLD302) materials had better stability than the other materials tested. These samples were used to test microefficacy of the release of hypoclorous acid vapors. The details of the tests were as follows: 10 uL of bacterial suspension (5% fetal bovine serum, 10⁸ S. aureus CFU/mL) was innoculated onto a 1 inch square glass slide. The slide was then dried at 35° F. for 30 minutes under sterile conditions. After the slides were dry, they were transferred into a 3.07 L Glad® container containing a petri dish (100×150 mm) with a bleach containing product. The weight, height, and concentration of the bleach containing products were recorded. The containers were closed and allowed to sit at room temperature for 3 hours after which the samples were removed aseptically. The samples were placed in D/E broth and vortexed for 30 minutes. 1 mL of this solution was then transferred into 9 mL of Butterfields buffer and vortexed. The solution was then diluted down as necessary and added to sterile petri dishes containing TSA. The dishes were incubated for 24 to 48 hours and then analyzed for the number of bacterial colonies. The results of the microefficacy testing is as follows: Samples containing silica, either CE0506, Aerogel® OGD303, or med pore Grace grade 59 and neat dilute bleach solution were tested for efficacy. All samples contained 40 g of bleach solution that was 195 ppm at pH 5.5 (adjusted with succinic acid). The control in the test was a Glad® container containing the innoculated glass slides with no bleach product. The glass slides were determined to have an average of 6×10⁶ CFU/mL before the test and the control slides had an average of 5×10⁶ CFU/mL after the experiment. All other slides showed complete kill after being exposed to the bleach samples for 3 hours in the closed Glad® containers. These results were further confirmed by looking at the color of the D/E broth which was yellow for the control samples (indicating bacterial growth) and purple for the bleach containing samples (indicating no bacterial growth). Results from the in vivo testing suggest that the inventive compositions will reduce or prevent respiratory ailments caused by allergens and reduce or prevent allergies.

### Further Methods for Diluting Hypochlorite

The stability results for dilute hypochlorite solutions diluted with deionized distilled water and adjusted to pH 7 are given below in Table XX for several buffering systems and concentrations of approximately 40 ppm, 75 ppm, and 150 ppm sodium hypochlorite. Citric acid, an organic hydroxyl containing acid has poor stability with or without sodium dihydrogen phosphate. However, hypochlorite buffered with hydrochloric acid or 3,3-dimethylglutaric acid, which has no enolizable hydrogens has good stability.

Besides metal contaminants, the compositions may also be substantially free of certain organic contaminants, such as surfactants or alcohols or amino compounds, or thiol compounds, or hydroxyacids, or olefinic compounds or fragrances. In some cases the composition may be substantially free of organic acids with enolizable hydrogens. The compositions may also have a low concentration of inorganic salts of less than 0.3 g/L.

TABLE XX

| | | | % Remaining | | | |
|---|---|---|---|---|---|---|
| Storage at 120° F. | Initial | | 7 days | 14 days | 21 days | 28 days |
| NaOCl diluted from 3.9% | 42.3 | ppm | 20% | 3% | 1% | 1% |
| with deionized distilled water | | | | | | |
| and 0.1M Citric Acid to pH 7.01 | | | | | | |
| NaOCl diluted from 3.9% | 77.5 | ppm | 3% | 1% | 1% | 1% |
| with deionized distilled water | | | | | | |
| and 0.1M Citric Acid to pH 7.01 | | | | | | |
| NaOCl diluted from 3.9% | 148.1 | ppm | 1% | 0% | 0% | 0% |
| with deionized distilled water | | | | | | |
| and 0.1M Citric Acid to pH 7.02 | | | | | | |
| NaOCl diluted from 3.9% | 41.5 | ppm | 26% | 11% | 3% | 1% |
| with deionized distilled water | | | | | | |
| and 0.1M NaH₂PO₄ and 0.1M | | | | | | |
| Citric Acid to pH 7.03 | | | | | | |
| NaOCl diluted from 3.9% | 78.7 | ppm | 12% | 1% | 1% | 1% |
| with deionized distilled water | | | | | | |
| and 0.1M NaH₂PO₄ and 0.1M | | | | | | |
| Citric Acid to pH 7.01 | | | | | | |
| NaOCl diluted from 3.9% | 147.9 | ppm | 1% | 0% | 0% | 0% |
| with deionized distilled water | | | | | | |
| and 0.1M NaH₂PO₄ and 0.1M | | | | | | |
| Citric Acid to pH 7.03 | | | | | | |
| NaOCl diluted from 3.9% | 42.5 | ppm | 96% | 88% | 87% | 86% |
| with deionized distilled water | | | | | | |
| and 0.1M HCl to pH 7.03 | | | | | | |
| NaOCl diluted from 3.9% | 78.1 | ppm | 97% | 91% | 90% | 87% |
| with deionized distilled water | | | | | | |
| and 0.1M HCl to pH 7.02 | | | | | | |
| NaOCl diluted from 3.9% | 145.8 | ppm | 93% | 85% | 82% | 80% |
| with deionized distilled water | | | | | | |
| and 0.1M HCl to pH 7.02 | | | | | | |
| NaOCl diluted from 3.9% | 42.6 | ppm | | 87% | 83% | 82% |
| with deionized distilled water | | | | | | |
| and 0.1M dimethylglutaric | | | | | | |
| acid to pH 7.02 | | | | | | |
| NaOCl diluted from 3.9% | 77.9 | ppm. | | 90% | 84% | 80% |
| with deionized distilled water | | | | | | |
| and 0.1M dimethylglutaric | | | | | | |
| acid to pH.03 | | | | | | |
| NaOCl diluted from 3.9% | 149.5 | ppm | | 82% | 77% | 73% |
| with deionized distilled water | | | | | | |
| and 0.1M dimethylglutaric | | | | | | |
| acid to pH 7.01 | | | | | | |

### Thickened Dilute Hypochlorite

Four drops of a solution of 200 ppm hypochlorite at pH 7 thickened with various amounts of Laponite® were placed on a Bisque Tile with Aspergillus niger and the residence time for the drop measured with the tile in a horizontal orientation. The results are given in Table XXI. Solutions of approximately 200 ppm hypochlorite and different pH values were tested for stability and effectiveness at decolorizing Aspergillus niger on a Bisque Tile (10 is completely decolored, 1 is not decolored) and the results are given in Table XXII.

TABLE XXI

| % Laponite ® | Residence time on horizontal tile (min) |
|---|---|
| 0 | 0.5 |
| 0.25 | 2 |
| 0.5 | 3 |
| 0.75 | 4.5 |
| 1 | 6 |

TABLE XXII

| | Stability vs. | Thickened | Unthickened |
|---|---|---|---|
| | Unthickened at 11 | Decolorization of | Decolorization of |
| pH | days and 120° F. | Aspergillus niger | Aspergillus niger |
| 5 | 79% | 7 | 3 |
| 7 | 94% | 7 | 3 |
| 9 | 98% | 8 | 2 |
| 11 | 100% | 3 | 1 |

Solutions of 200 ppm hypochlorite were thickened with Laponite® with added buffers to give viscous liquids or gels, as shown in Table XXIII. Gels were also formed with the addition of acetic acid or hydrochloric acid.

TABLE XXIII

| Buffer | Wt. % | pH | gel |
|---|---|---|---|
| Boric acid | 0.21 | 8.5 | Yes |
| Boric acid | 0.41 | 8.4 | Yes |
| Succinic acid | 0.01 | 9.2 | Yes |
| Succinic acid | 0.04 | 8.2 | Viscous liquid |

Various thickeners were tested at 1% concentration and pH 7 for their effect on the stability of dilute hypochlorite and results are shown in Table XXIV. By comparison, surfactants that are normally considered stable to hypochlorite, such as sodium alkylbenzenesulfonate, trimethylC₁₆ ammonium chloride, sodium lauryl sulfate, and sodium octyl sulfonate, were less stable than Vangel ES®. At higher pH values, the thickener will likely have higher stability.

TABLE XXIV

| | | % NaOCl remaining after 8 |
|---|---|---|
| Thickener | Type | days at 120° F. |
| None | | 84.8% |
| Laponite R ® | Synthetic silicate hectorite | 84.5 |
| | clay | |
| Vangel ES ® | Mg aluminum silicate | 63.0 |
| | smectite clay | |
| Vangel B ® | Mg aluminum silicate | 0 |
| | smectite clay | |
| Catapal D ® | alumina | 0 |

Laponite® was also observed to improve the wetting behavior of dilute hypochlorite compositions. When a solution of 200 ppm hypochlorite at pH 7 thickened with 0.5% or 1% by weight Laponite® was sprayed onto a mirror and then wiped, it was found to dry evenly, whereas the solution without Laponite® was found to dry with droplets and fisheyes. Additionally, the mirror treated with the Laponite® containing hypochlorite solution left a surface that easily rewet, so that water spread evenly on the surface. The solution without Laponite® did not leave a surface that easily rewet, to that water runs off unevenly from the surface.

Removal of Allergens from the ir

Inhalation of airborne allergens is the primary route to trigger allergic response. Therefore, it is desirable to be able to reduce allergen levels in the air directly. A spray of a dilute hypochlorite can not only reduce the airborne allergenic particles in the air but also denature or reduce allergenicity of the particles as well. In one example, house dust containing cat and dog allergens was continuously aerosolized into a 1 cubic meter chamber until a constant level of approximately 100 ug/m3 was achieved. This level is on the order of that known for normal activity in homes. Once a constant level was reached, a dilute hypochlorite mist (pH 7 and 85 ppm, pH 5.5 and 95 ppm) with particle sizes of approximately 60 um was sprayed into the chamber for 20 seconds delivering approximately 12 ml of hypochlorite solution. Then 3 sample pumps placed around the chamber containing filters were turned on pulling air through filters to collect remaining airborne dust. ELISA testing was done to compare the allergen levels in the dust with untreated controls. Reduction levels for cat allergen were 75% vs. no spray, and 43% reduction vs. water spray. Reduction levels for dog allergen were 85% vs. no spray, and 63% vs. water spray. A spray of dilute hypochlorite of larger particle size would be less effective at removing allergens from the air.

### Comparative Particle Size Distribution

The volume mean diameter D[4,3] in microns was measured for Inventive Product Containers (containing dilute hypochlorite) and Comparative commercial products using Malvern Mastersizer® Model S, Malvern Instruments, Malvern, Worcestershire, UK. The results are shown in Table XXV.

TABLE XXV

| Product | Mean Particle Size, um |
|---|---|
| Inventive Hard Surface Spray A | 130.6 |
| Inventive Hard Surface Spray B | 119.7 |
| Inventive Air or Soft Surface Spray C | 58.4 |
| Inventive Air or Soft Surface Spray D | 63.1 |
| Inventive Air or Soft Surface Aerosol E | 87.6 |
| Inventive Air or Soft Surface Aerosol F | 91.3 |
| Febreeze ® Original Spray | 235.3 |
| Febreeze ® Anti-Allergen Spray | 216.9 |

### Microefficacy

The inventive containers were filled with compositions containing 50 to 200 ppm hypochlorite at pH 5 to pH 8. Inventive Hard Surface Spray A was effective at sanitization of bacteria such as Escherichia coli, Salmonella choleraesuis, Staphylococcus aureus, Klebsiella pneumoniae, Streptococcus pneumoniae, and Proteus mirabilis on hard surfaces such as glass. Inventive Air or Soft Surface Aerosol E was effective at sanitization of Staphylococcus aureus and Klebsiella pneumoniae on soft surfaces such as cotton.

### Dye Damage and Particle Size

Although these products are generally safe for use, dye damage can occur on select dyed fabrics that are very susceptible to color change. Generally, as the mean particle size increases, so does the amount of dye-damage. The inventive containers were filled with compositions containing 50 to 200 ppm hypochlorite at pH 5 to pH 8. In one example, nineteen fabrics were treated with various Inventive Soft Surface Sprays and Aerosols. Color change (ΔE) was measured at regular intervals over the course of a multi-treatment study, representing long-term use of the sprays and aerosols. The average ΔE correlates to the mean particle size of the sprays and aerosols. For a set of Inventive Soft Surface Sprays and Aerosols containing the same amount of active ingredient, the Pearson Correlation of mean particle size and ΔE was 0.922, indicating a very strong positive correlation. (A value of 0 indicates there is no correlation, and a value of 1 indicates the maximum correlation possible.) The ΔE for an Inventive Hard Surface Spray was 3.9 (particle size of 119.7 um) and the ΔE for an Inventive Air or Soft Surface Spray was 2.0 (particle size of 63.1 um).

While various patents have been incorporated herein by reference, to the extent there is any inconsistency between incorporated material and that of the written specification, the written specification shall control. In addition, while the invention has been described inherein in considerable detail with respect to specific embodiments thereof, it will be apparent to provide those skilled in the art that various alterations, modifications and other changes may be made to with information relevant to apply the novel principles and to construct and use such specialized components as are required. However, it is to be understood that the invention can be carried out by different equipment, materials and devices, and that various modifications, both as to the equipment and operating procedures, can be accomplished without departing from the spirit and scope of the present invention. It is therefore intended that the claims cover all such modifications, alterations and other changes encompassed by the appended claims.

We claim:

1. A method for producing a stable dilute composition, said composition selected from the group consisting of hypohalous acid, hypohalous acid salt, and combinations thereof, said method comprising the steps of:
preparing a first solution having an active halogen content of greater than about 0.5% as available chlorine; and
diluting said first solution with purified water to give a second solution;
wherein said second solution has an available chlorine concentration of between 40 ppm to about 400 ppm;
wherein said second solution retains at least 50% of the available chlorine concentration at a storage temperature of 120° F. over 27 days;
wherein said stable dilute composition does not contain additives selected from the group consisting of surfactants, alcohols, hydroxyacids, fragrances or combinations thereof.
wherein said second solution has an available chlorine concentration of between 40 ppm to about 400 ppm;
wherein said second solution retains at least 50% of the available chlorine concentration at a storage temperature of 120° F. over 27 days;
wherein said stable dilute composition does not contain additives selected from the group consisting of surfactants, alcohols, hydroxyacids, fragrances or combinations thereof.

2. The method of claim 1, wherein said second solution additionally comprises a pH adjusting agent selected from the group consisting of carbon dioxide, alkali metal carbonate, alkali metal bicarbonate, alkali metal silicates, alkali metal hydroxide, alkali phosphate salt, alkaline earth phosphate salt, alkali borate salt, hydrochloric acid, nitric acid, sulfuric acid, alkali metal hydrogen sulfate, organic sulfonic acids, sulfamic acid, and mixtures thereof.

3. The method of claim 1, wherein said second solution additionally comprises a pH adjusting agent selected from a carboxylic acid having no hydroxyl groups or olefinic groups.

4. The method of claim 1, wherein said second solution has a salt concentration of less than 0.3 g/L.

5. A package for dilute hypochlorite comprising:
a container;
a label; and
a composition within the container, said composition selected from the group consisting of hypohalous acid, hypohalous acid salt, and combinations thereof,
wherein said composition has an available chlorine concentration of between 1.0 ppm to about 1200 ppm;
wherein said container is selected from the group consisting of a trigger sprayer, a bag-in-can device, a plastic aerosol container, a dual delivery container, a dual chambered device, an expandable chamber device, a precompression trigger sprayer, a mechanically pressurized device, an ultrasonic sprayer, and combinations thereof.

6. The package of claim 5, wherein said container comprises a multilayer container comprising:
an inner layer;
an outer layer;
an optional intermediate layer;
wherein at least one of said outer layer or said intermediate layer comprises an additive selected from the group consisting of opacifiers, colorants, UV inhibitors and combinations thereof; and
wherein said inner layer comprises a substantially lower concentration of one of said additives compared to said outer layer or compared to said optional intermediate layer.

7. The package of claim 5, wherein said label comprises an additive selected from the group consisting of an opacifier, a colorant, a UV inhibitor, and combinations thereof.

8. A system for mold or allergen removal comprising:
a detection device for mold or allergen; and
a treatment device for mold or allergen.

9. The system of claim 8, wherein the system additionally comprises instructions for mold or allergen treatment, said instructions comprising the steps of:
using a means for identifying the existence of mold or allergen;
applying a composition for the treatment of mold or allergen; and
optionally, providing educational materials about mold or allergen;
optionally, providing guidelines for how to take care of the mold or allergen problem based on the results of the detection device;
optionally, measuring the result of the mold or allergen treatment optionally, providing a treatment for inhibiting future mold or allergen.

10. The system of claim 9, wherein the treatment device comprises a mold or allergen deactivating agent selected from the group consisting of a hypohalous acid, a hypohalous acid salt, and a combination thereof; and wherein the set of instructions comprises instructions to contact targets selected from the group consisting of hard surfaces, soft surfaces, and air with said liquid composition in a form selected from a group consisting of neat, diluted, and a combination thereof to accomplish a result selected from the group consisting of, to prevent allergic or mold response, to prevent illness, and a combination thereof.

11. A powder composition comprising:
greater than 10% water;
a compound selected from the group consisting of hypochlorite, hypochlorous acid, and combinations thereof; and
silica.

12. The powder of claim 11, wherein said powder comprises greater than 0.5% of said hypohalite compound selected from the group consisting of hypochlorite, hypochlorous acid, and combinations thereof.

13. The powder of claim 11, wherein said powder comprises less than 0.5% of said hypohalite compound selected from the group consisting of hypo chlorite, hypochlorous acid, and combinations thereof.

14. A method of controlling microbiological contaminants in a confined space comprising the steps of:
optionally, placing an object containing a microbiological contaminant in the confined space
placing a composition comprising a source of hypohalous acid in the confined space;
allowing hypohalous acid vapor from the source of hypohalous acid to control microbiological contaminants.

15. The method of claim 14, wherein the confined space is a sealable container or room.

16. The method of claim 14, wherein the composition is in a form selected from the group consisting of a solid, a liquid, a gel, or a combination thereof.

17. The method of claim 14, wherein the composition is within a permeable container.

18. The method of claim 15, wherein the permeable container is a dispersion device selected for the group consisting of pouches, humidifiers, fans, sprayers, dispersers, and combinations thereof.

19. The method of claim 16, wherein the composition is a solid comprising a carrier and an oxidant.

20. the method of claim 14, wherein the source of hypohalous acid also generates halogen gas vapor and the halogen gas vapor is reduced to less than 1% of the hypohalous acid concentration.

21. The method of claim 20, wherein the halogen gas vapor is reduce by a means selected from the group consisting of a dessicant, a membrane, a filter, controlling the composition pH, controlling the concentration of hypohalous acid or hypohalous acid salt with the composition, using mechanical dispersion, or combinations thereof.
